SEQUENCE LISTING (136 sequences)

SEQ ID NO: 1 - protein, 8 aa, synthetic construct
CAVQKLVF

SEQ ID NO: 2 - protein, 13 aa, synthetic construct
CAAAGGGADGLTF

SEQ ID NO: 3 - protein, 10 aa, synthetic construct
CAVNNARLMF

SEQ ID NO: 4 - protein, 12 aa, synthetic construct
CAVNTGGFKTIF

SEQ ID NO: 5 - protein, 12 aa, synthetic construct
CAAGGGADGLTF

SEQ ID NO: 6 - protein, 11 aa, synthetic construct
CALGFGNVLHC

SEQ ID NO: 7 - protein, 11 aa, synthetic construct
CAVNIGARLMF

SEQ ID NO: 8 - protein, 12 aa, synthetic construct
CAVASDGQKLLF

SEQ ID NO: 9 - protein, 13 aa, synthetic construct
CAVYGGSQGNLIF

SEQ ID NO: 10 - protein, 8 aa, synthetic construct
CAVNQFYF

SEQ ID NO: 11 - protein, 12 aa, synthetic construct
CAGGGGADGLTF

SEQ ID NO: 12 - protein, 10 aa, synthetic construct
CAANNARLMF

SEQ ID NO: 13 - protein, 11 aa, synthetic construct
CAVNQGGKLIF

SEQ ID NO: 14 - protein, 14 aa, synthetic construct
CAVNSGGGADGLTF

SEQ ID NO: 15 - protein, 13 aa, synthetic construct
CAVSIGFGNVLHC

SEQ ID NO: 16 - protein, 12 aa, synthetic construct
CAVHTGGFKTIF

SEQ ID NO: 17 - protein, 12 aa, synthetic construct
CAVDNQGGKLIF

SEQ ID NO: 18 - protein, 13 aa, synthetic construct
CAVSIGFGNVLHC

SEQ ID NO: 19 - protein, 13 aa, synthetic construct
CAMSVGFGNVLHC

SEQ ID NO: 20 - protein, 12 aa, synthetic construct
CAALYNTDKLIF

SEQ ID NO: 21 - protein, 11 aa, synthetic construct
CAARGAQKLVF

SEQ ID NO: 22 - protein, 12 aa, synthetic construct
CAATLYNNDMRF

SEQ ID NO: 23 - protein, 13 aa, synthetic construct
CVVSASNGNKLVF

SEQ ID NO: 24 - protein, 13 aa, synthetic construct
CAYLVGSNYQLIW

SEQ ID NO: 25 - protein, 14 aa, synthetic construct
CAFMKPNTGNQFYF

SEQ ID NO: 26 - protein, 15 aa, synthetic construct
CASSPYSGSGETQYF

SEQ ID NO: 27 - protein, 13 aa, synthetic construct
CASSFAGTDTQYF

SEQ ID NO: 28 - protein, 16 aa, synthetic construct
CASSYSFTEATYEQYF

SEQ ID NO: 29 - protein, 14 aa, synthetic construct
CASSLTGLGQPQHF

SEQ ID NO: 30 - protein, 14 aa, synthetic construct
CASSYRGLGQPQHF

SEQ ID NO: 31 - protein, 14 aa, synthetic construct
CASSATVLGTEAFF

SEQ ID NO: 32 - protein, 14 aa, synthetic construct
CSVEESSGIYEQYF

SEQ ID NO: 33 - protein, 14 aa, synthetic construct
CASSSAGMGQPQHF

SEQ ID NO: 34 - protein, 14 aa, synthetic construct
CAWSSQGLGQPQHF

SEQ ID NO: 35 - protein, 13 aa, synthetic construct
CASTLGTGDGYTF

SEQ ID NO: 36 - protein, 14 aa, synthetic construct
CSAPQTGLGQPQHF

SEQ ID NO: 37 - protein, 15 aa, synthetic construct
CASSFGPLANYGYTF

SEQ ID NO: 38 - protein, 15 aa, synthetic construct
CASSQGLAGSNEQFF

SEQ ID NO: 39 - protein, 14 aa, synthetic construct
CSATGLAGLGEQFF

SEQ ID NO: 40 - protein, 14 aa, synthetic construct
CASSLTGLGQPQHF

SEQ ID NO: 41 - protein, 14 aa, synthetic construct
CASSPGTIYQPQHF

SEQ ID NO: 42 - protein, 15 aa, synthetic construct
CASAGGALVGYGYTF

SEQ ID NO: 43 - protein, 14 aa, synthetic construct
CASSFQGLGQPQHF

SEQ ID NO: 44 - protein, 15 aa, synthetic construct
CASTLGGTLGSPLHF

SEQ ID NO: 45 - protein, 13 aa, synthetic construct
CASTLGQGWEQYF

SEQ ID NO: 46 - protein, 14 aa, synthetic construct
CASSLQGLGQPQHF

SEQ ID NO: 47 - protein, 15 aa, synthetic construct
CASSLSVISTGELFF

SEQ ID NO: 48 - protein, 15 aa, synthetic construct
CASGKGQIFAGELFF

SEQ ID NO: 49 - protein, 14 aa, synthetic construct
CATSGLAGAYEQYF

SEQ ID NO: 50 - protein, 15 aa, synthetic construct
CASSEYTSGNQPQHF

SEQ ID NO: 51 - protein, 14 aa, synthetic construct
CASSPAGLGQPQHF

SEQ ID NO: 52 - protein, 17 aa, synthetic construct
CASSPDPFGLAGNEQFF

SEQ ID NO: 53 - protein, 13 aa, synthetic construct
CASSWAGPVEQYF

SEQ ID NO: 54 - protein, 14 aa, synthetic construct
CASSLGLAGNEQYF

SEQ ID NO: 55 - protein, 13 aa, synthetic construct
CASSSGLTDTQYF

SEQ ID NO: 56 - protein, 15 aa, synthetic construct
CASSLGIISGQPQHF

SEQ ID NO: 57 - protein, 14 aa, synthetic construct
CASSLGGGGYEQYF

SEQ ID NO: 58 - protein, 14 aa, synthetic construct
CAISPGEGTDTQYF

SEQ ID NO: 59 - protein, 15 aa, synthetic construct
CASSYTGTLSYEQYF

SEQ ID NO: 60 - protein, 14 aa, synthetic construct
CASSYVGLGSPLHF

SEQ ID NO: 61 - protein, 13 aa, synthetic construct
CATSRDMDPEQYF

SEQ ID NO: 62 - protein, 13 aa, synthetic construct
CASSLGGADTQYF

SEQ ID NO: 63 - protein, 14 aa, synthetic construct
CASSFLQGTGELFF

SEQ ID NO: 64 - protein, 14 aa, synthetic construct
CASRVGILSGELFF

SEQ ID NO: 65 - protein, 13 aa, synthetic construct
CATSRDRGWEQFF

SEQ ID NO: 66 - protein, 12 aa, synthetic construct
CASSLSGAEAFF

SEQ ID NO: 67 - protein, 14 aa, synthetic construct
CASSPSWAHETQYF

SEQ ID NO: 68 - protein, 15 aa, synthetic construct
CASSPSGTVYEKLFF

SEQ ID NO: 69 - protein, 13 aa, synthetic construct
CSASEGIGQPQHF

SEQ ID NO: 70 - protein, 14 aa, synthetic construct
CASSPGLTANVLTF

SEQ ID NO: 71 - protein, 15 aa, synthetic construct
CASSQEQLAGPEQYF

SEQ ID NO: 72 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 73 - protein, 180 aa, Homo sapiens
MASTSYDYCRVPMEDGDKRCKLLLGIGILVLLIIVILGVPLIIFTIKANSEACRDGLRAVMECRNVTHLLQQELTEAQKGFQDVEAQAATCNHTVMALMASLDAEKAQGQKKVEELEGEITTLNHKLQDASAEVERLRRENQVLSVRIADKKYYPSSQDSSSAAAPQLLIVLLGLSALLQ

SEQ ID NO: 74 - protein, 599 aa, Homo sapiens
MMNKLYIGNLSPAVTADDLRQLFGDRKLPLAGQVLLKSGYAFVDYPDQNWAIRAIETLSGKVELHGKIMEVDYSVSKKLRSRKIQIRNIPPHLQWEVLDGLLAQYGTVENVEQVNTDTETAVVNVTYATREEAKIAMEKLSGHQFENYSFKISYIPDEEVSSPSPPQRAQRGDHSSREQGHAPGGTSQARQIDFPLRILVPTQFVGAIIGKEGLTIKNITKQTQSRVDIHRKENSGAAEKPVTIHATPEGTSEACRMILEIMQKEADETKLAEEIPLKILAHNGLVGRLIGKEGRNLKKIEHETGTKITISSLQDLSIYNPERTITVKGTVEACASAEIEIMKKLREAFENDMLAVNQQANLIPGLNLSALGIFSTGLSVLSPPAGPRGAPPAAPYHPFTTHSGYFSSLYPHHQFGPFPHHHSYPEQEIVNLFIPTQAVGAIIGKKGAHIKQLARFAGASIKIAPAEGPDVSERMVIITGPPEAQFKAQGRIFGKLKEENFFNPKEEVKLEAHIRVPSSTAGRVIGKGGKTVNELQNLTSAEVIVPRDQTPDENEEVIVRIIGHFFASQTAQRKIREIVQQVKQQEQKYPQGVASQRSK

SEQ ID NO: 75 - protein, 10 aa, Homo sapiens
EAAGIGILTV

SEQ ID NO: 76 - protein, 10 aa, Homo sapiens
LLLGIGILVL

SEQ ID NO: 77 - protein, 10 aa, Homo sapiens
NLSALGIFST

SEQ ID NO: 78 - protein, 201 aa, synthetic construct
MQKEVEQNSGPLSVPEGAIASLNCTYSDRGSQSFFWYRQYSGKSPELIMSIYSNGDKEDGRFTAQLNKASQYVSLLIRDSQPSDSATYLCAVQKLVFGTGTRLLVSPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 79 - protein, 246 aa, synthetic construct
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSFTEATYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 80 - DNA, 804 bp, synthetic construct
  source 1..804: mol_type = other DNA
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgcagaaacttgtatttggaactggcacccgacttctggtcagtccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 81 - DNA, 939 bp, synthetic construct
  source 1..939: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttactcctttactgaggcgacctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 82 - protein, 6 aa, synthetic construct
DRGSQS

SEQ ID NO: 83 - protein, 6 aa, synthetic construct
IYSNGD

SEQ ID NO: 84 - protein, 5 aa, synthetic construct
MNHEY

SEQ ID NO: 85 - protein, 5 aa, synthetic construct
MDHEN

SEQ ID NO: 86 - protein, 5 aa, synthetic construct
LNHDA

SEQ ID NO: 87 - protein, 5 aa, synthetic construct
SQVTM

SEQ ID NO: 88 - protein, 6 aa, synthetic construct
GTSNPN

SEQ ID NO: 89 - protein, 6 aa, synthetic construct
DFQATT

SEQ ID NO: 90 - protein, 5 aa, synthetic construct
LGHNA

SEQ ID NO: 91 - protein, 5 aa, synthetic construct
SGHDT

SEQ ID NO: 92 - protein, 5 aa, synthetic construct
SGHTA

SEQ ID NO: 93 - protein, 5 aa, synthetic construct
MNHNS

SEQ ID NO: 94 - protein, 5 aa, synthetic construct
SGHDY

SEQ ID NO: 95 - protein, 5 aa, synthetic construct
PRHDT

SEQ ID NO: 96 - protein, 5 aa, synthetic construct
LNHNV

SEQ ID NO: 97 - protein, 6 aa, synthetic construct
SVGAGI

SEQ ID NO: 98 - protein, 6 aa, synthetic construct
SMNVEV

SEQ ID NO: 99 - protein, 6 aa, synthetic construct
SYDVKM

SEQ ID NO: 100 - protein, 6 aa, synthetic construct
SQIVND

SEQ ID NO: 101 - protein, 7 aa, synthetic construct
ANQGSEA

SEQ ID NO: 102 - protein, 5 aa, synthetic construct
SVGIG

SEQ ID NO: 103 - protein, 7 aa, synthetic construct
SNEGSKA

SEQ ID NO: 104 - protein, 6 aa, synthetic construct
YNFKEQ

SEQ ID NO: 105 - protein, 6 aa, synthetic construct
YYEEEE

SEQ ID NO: 106 - protein, 6 aa, synthetic construct
YSLEER

SEQ ID NO: 107 - protein, 6 aa, synthetic construct
SVGEGT

SEQ ID NO: 108 - protein, 6 aa, synthetic construct
FQGNSA

SEQ ID NO: 109 - protein, 6 aa, synthetic construct
SASEGT

SEQ ID NO: 110 - protein, 5 aa, synthetic construct
ENHRY

SEQ ID NO: 111 - protein, 6 aa, synthetic construct
FNNNVP

SEQ ID NO: 112 - protein, 6 aa, synthetic construct
FQGTGA

SEQ ID NO: 113 - protein, 6 aa, synthetic construct
FYEKMQ

SEQ ID NO: 114 - protein, 6 aa, synthetic construct
SYGVKD

SEQ ID NO: 115 - protein, 6 aa, synthetic construct
YYDKDF

SEQ ID NO: 116 - protein, 11 aa, synthetic construct
CAVNVAGKSTF

SEQ ID NO: 117 - protein, 12 aa, synthetic construct
CAVGAGGFKTIF

SEQ ID NO: 118 - protein, 12 aa, synthetic construct
CAVNYEDDKIIF

SEQ ID NO: 119 - protein, 12 aa, synthetic construct
CAVSDNGNKLVF

SEQ ID NO: 120 - protein, 11 aa, synthetic construct
CGSNTGNQFYF

SEQ ID NO: 121 - protein, 15 aa, synthetic construct
CAWSETGLGTGELFF

SEQ ID NO: 122 - protein, 15 aa, synthetic construct
CASSESPLSGNEQYF

SEQ ID NO: 123 - protein, 15 aa, synthetic construct
CASSVGVQGSWEQYF

SEQ ID NO: 124 - protein, 18 aa, synthetic construct
CASSQGVLVIAGVGEQYF

SEQ ID NO: 125 - protein, 15 aa, synthetic construct
CASSEAVLYVDTQYF

SEQ ID NO: 126 - protein, 13 aa, synthetic construct
CASSFAGTDTQYF

SEQ ID NO: 127 - protein, 5 aa, synthetic construct
SNHLY

SEQ ID NO: 128 - protein, 6 aa, synthetic construct
FYNNEI

SEQ ID NO: 129 - protein, 5 aa, synthetic construct
SGDLS

SEQ ID NO: 130 - protein, 6 aa, synthetic construct
HYYNGE

SEQ ID NO: 131 - protein, 6 aa, synthetic construct
NSAFQY

SEQ ID NO: 132 - protein, 6 aa, synthetic construct
TYSSGN

SEQ ID NO: 133 - protein, 6 aa, synthetic construct
NTAFDY

SEQ ID NO: 134 - protein, 7 aa, synthetic construct
IRPDVSE

SEQ ID NO: 135 - protein, 6 aa, synthetic construct
SSVSVY

SEQ ID NO: 136 - protein, 8 aa, synthetic construct
YLSGSTLV

(19) United States
(12) Patent Application Publication
(10) Pub. No.: US 2026/0053920 A1
(43) Pub. Date: Feb. 26, 2026

(54) NOVEL TCRs

(71) Applicant: University College Cardiff Consultants Limited, Cardiff, Wales (GB)

(72) Inventors: Andrew SEWELL, Cardiff, Wales (GB); Garry DOLTON, Cardiff, Wales (GB)

(21) Appl. No.: 19/099,857

(22) PCT Filed: Aug. 1, 2023
(86) PCT No.: PCT/GB2023/052026

§ 371 Date: Jan. 30, 2025

(30) Foreign Application Priority Data
Aug. 1, 2022 (GB) 2211194.2

(51) Int. Cl. A61K 40/11; A61K 40/32; A61K 40/42; A61P 35/00; C07K 14/725; C07K 16/30

(52) U.S. Cl. CPC A61K 40/11; A61K 40/32; A61K 40/4272; A61P 35/00; C07K 14/7051; C07K 16/3053; A61K 2239/28

(57) ABSTRACT

There is provided inter alia an isolated anti-cancer T-cell receptor (TCR), or anti-cancer binding fragment thereof, that binds to a plurality of cancer polypeptide antigens or antigenic peptide fragments thereof wherein said cancer polypeptide antigens or antigenic peptide fragments thereof are distinct from each other and are presented at the cell surface of one or more than one type of cancer cell.

### FIELD OF THE INVENTION

The present invention relates to anti-cancer TCRs and T-cells comprising said TCRs that can target multiple different tumor types via HLA A presented epitopes of Melan A, BST2 and IMP2 by targeting multiple different cancer polypeptide antigens, more specifically cancer polypeptide antigens which share the x-x-x-A/G-I/L-G-I-x-x-x recognition motif, especially where the epitope sequences comprise the sequences EAAGIGILTV, LLLGIGILVL, and NLSALGIFST of Melan A, BST2 and IMP2 respectively. The present invention further relates to a vector encoding said TCR; a bispecific comprising said TCR; a pharmaceutical composition comprising said TCR or T-cell or bispecific, use of said anti-cancer TCR, T-cell, vector, pharmaceutical composition, bispecific to treat cancer; a method of treating cancer using said anti-cancer TCR, T-cell, vector, pharmaceutical composition, bispecific; and a combination therapeutic for the treatment of cancer comprising said anti-cancer TCR, T-cell, vector, pharmaceutical composition, or bispecific.

### BACKGROUND

Cytotoxic T lymphocytes (CTLs) detect and destroy cells containing anomalies in protein expression caused by intracellular pathogens or cellular transformation, such CTLs recognise individual cancer peptides through their cognate T-cell receptor. The ability of CTL to distinguish the proteome of healthy cells from that of cancer cells, and to destroy the latter, has been exploited using checkpoint blockade and TIL (tumour infiltrating lymphocytes) therapy. Generally, it appears that a single TCR recognises a single cancer antigenic peptide typically when presented at the cell surface in the context of human leukocyte antigen (HLA) class I or class II molecule. We have previously demonstrated the discovery of T cells which can recognise multiple, distinct peptides that are derived from different cancer antigens when presented at the cell surface in the context of the same human leukocyte antigen, WO2020002899. In these T cells a single TCR has the ability to recognise multiple and distinct cancer antigens when presented at the cell surface in the context of the same human leukocyte antigen (HLA) class I molecule at the surface of the same cancer cell. It therefore appears that some rare T-cells (“multipronged T-cells”) are capable of recognising a range of individual cancer antigenic peptides through their cognate T-cell receptor, advantageously these “multipronged T-cells” can recognise and attack cancer cells via more than one antigen and thereby vastly reduce the chances of immune escape by cancer cells. It is recognised that there is a need to provide better and safer ways of treating or eradicating cancer and immunotherapy using the body's natural defence systems is acknowledged to be safer than chemical intervention.

The anti-cancer TCRs and T-cells we have identified herein have the above advantageous characteristics in that they are effective against more than one type of cancer thus safeguarding against a cancer evading the effectiveness of the immune system. The disclosed multi-epitope targeting T-cells have the capability to attack cancer in several ways simultaneously and exhibit superior recognition of cancer cells compared to conventional T-cell recognition of individual epitopes making them advantageous candidates for cancer immunotherapy.

### SUMMARY OF THE INVENTION

The T-cells of the clones identified by the Inventors herein have the aforementioned advantageous characteristics in that they target multiple cancer epitopes and are effective against many types of cancer.

According to the present invention, there is provided an isolated anti-cancer T-cell receptor (TCR), or anti-cancer binding fragment thereof, that binds to a plurality of cancer polypeptide antigens or antigenic peptide fragments thereof wherein said cancer polypeptide antigens or antigenic peptide fragments thereof are distinct from each other and are presented at the cell surface of one or more than one type of cancer cell, particularly wherein, the cancer polypeptide antigen or antigenic peptide fragment thereof comprises the amino acid sequence motif X-A/G-I/L-G-I-X wherein X is any amino acid, wherein I/L indicates that either amino acid I or L may be present at the residue position (similarly for A/G) and wherein said isolated anti-cancer T-cell receptor (TCR), or anti-cancer binding fragment thereof binds to an amino acid sequence comprising said sequence motif.

The present invention further provides a polynucleotide encoding the anti-cancer T-cell receptor (TCR), or anti-cancer binding fragment thereof, according to the invention and a vector for delivery of a polynucleotide to cells comprising a polynucleotide according to the invention.

The present invention further provides an immune cell or an immune cell clone, particularly a T-cell or a T-cell clone expressing the anti-cancer T-cell receptor (TCR), or anti-cancer binding fragment thereof according to the invention, i.e. a modified immune cell or immune cell clone.

The present invention further provides an ex vivo process comprising (i) obtaining immune cells, particularly T-cells from a patient, (ii) optionally expanding the immune cells (iii) introducing a heterologous polynucleotide according to the invention or a vector according to the invention into the immune cells, particularly T-cells to produce modified immune cells, particularly T-cells which express a anti-cancer T-cell receptor (TCR), or anti-cancer binding fragment thereof according to the invention; and (iii) reintroducing said modified immune cells, particularly T-cells into the patient.

The present invention further provides a method of treatment of cancer comprising administering to a patient in need thereof transduced or modified immune cells, particularly T-cells wherein the immune cells are immune cells that have been obtained from said patient and a heterologous polynucleotide according to the invention or a vector according to the invention has been introduced into the immune cells, particularly T-cells, such that the immune cells, particularly T-cells express an anti-cancer T-cell receptor (TCR), or anti-cancer binding fragment thereof according to the invention.

The present invention further provides a pharmaceutical composition comprising the anti-cancer T-cell receptor (TCR), or anti-cancer binding fragment thereof according to the invention, the polynucleotide according to the invention, a vector according to the invention, the immune cell or the immune cell clone (such as the T-cell or the T-cell clone) according to the invention and a pharmaceutically acceptable carrier.

The present invention further provides a bispecific construct comprising the anti-cancer T-cell receptor (TCR), or anti-cancer binding fragment thereof according to the invention and an immune cell activating component or ligand that binds to and activates an immune cell.

The present invention further provides a fusion protein comprising the anti-cancer T-cell receptor (TCR), or anti-cancer binding fragment thereof according to the present invention and a heterologous protein.

The present invention further provides a method of treating cancer in a subject comprising administering a therapeutically effective amount of the immune cell or the immune cell clone (such as the T-cell or T-cell clone) according to the invention, pharmaceutical composition according to the invention, bispecific construct according to the invention or fusion protein according to the invention, to the subject.

The present invention further provides a pharmaceutical composition comprising:
a) the immune cell or the immune cell clone (such as the T-cell or the T-cell clone) according to the invention, the pharmaceutical composition according to the invention, bispecific construct according to the invention or the fusion protein according to the invention; and
b) an anti-cancer agent.

### BRIEF DESCRIPTION OF THE FIGURES

FIG. 1. Cytotoxicity of MM909.24 TIL against autologous melanoma assessed by chromium release assay after 4 and 18 hr incubation (Mean and SEM).

FIG. 2. TAPI-0 assay (4 h) with the MM909.24 TIL co-incubated with a panel of wild-type HLA A2+ and HLA A2neg (negative) melanoma cell lines. Gates were set on single lymphocytes and live CD8+CD3+ cells.

FIG. 3. Intracellular staining following 5 h co-incubation of M909.24 TIL with autologous melanoma, which had been previously incubated with anti-HLA A2 or anti-HLA A1 monoclonal antibodies as indicated in X-axis.

FIG. 4. TAPI-0 assay (4 h) of M909.24 TIL with wild type (WT) or CRISPR/Cas9 HLA A2 knockout (KO) autologous melanoma. Percentage from duplicate conditions of cancer-reactive (TNF+CD107a+) cells found within the TIL population is indicated above each panel. Gates set on single lymphocytes and live CD8+CD3+ cells.

FIG. 5. TAPI-0 assay (5 h) of M909.24 TIL with a panel of HLA A2+ and HLA A2neg (negative) cancer cell lines and C1R cells (cell line type indicated by the key). Performed over four assays with reactivity normalized to WT autologous melanoma cells. Genetic modifications according to the key. Data represents with error bars depicting SEM.

FIG. 6. Two day killing assay of MEL8 T-cell clone following co-incubation with a panel of HLA A2+ and HLA A2neg cancer cell lines and C1Rs. Data shows the mean with error bars depicting SEM.

FIG. 7. Overnight cytokine release of MEL8 T-cell clone following co-incubation with a panel of HLA A2+ and HLA A2neg cancer cell lines and C1Rs. Data shows the mean with error bars depicting SEM.

FIG. 8. MEL8 T-cell clone specificity, tetramer staining (left) and overnight peptide activation followed by MIP-1beta ELISA (right) using Melan-A26-35 (EAAGIGILTV) peptide & HLA A2-ALWGPDPAAA (preproinsulin 15-24) used as an irrelevant tetramer.

FIG. 9. MEL8 T-cell clone cytotoxicity after 5 h of WT and HLA A2 CRISPR/Cas9 knock out autologous melanoma assessed by chromium release assay. Effector/T-cell:melanoma cell line ratios according to the x-axis.

FIG. 10. Peptide length ‘sizing-scan’ of MEL8 T-cell clone measuring MIP-1beta by ELISA following overnight co-incubation with peptide mixtures and T2 antigen presenting cells.

FIG. 11. Candidate peptide sequences predicted using selected PS-CPL and CAntigen database for MEL8 recognition. Peptides are ranked/scored for likelihood of recognition by MEL8.

FIG. 12. EC50 of MEL8 peptide activation assessed by MIP-1beta ELISA after overnight co-incubation with the indicated peptides from (D). Mean of duplicates and standard error is shown.

FIG. 13. Tetramer staining of MEL8 and three other clones MEL 11, 23, 31 (unique TCRs) from the TIL of patient MM909.24: HLA A2 Melan A, BST222-31, and IMP2367-376. Gates were set on single lymphocytes and live CD3+CD8+ cells. Mean Fluorescence Intensities (MFIs) are shown for each stain. Irrelevant tetramer=HLA A2 ILAKFLHWL (hTERT540-548).

FIG. 14. Peptide (10-6 M) activation of MEL11, MEL25 and MEL31 CD8+ T-cell clones with Melan A, BST2 and IMP2 peptides. Overnight incubation and MIP-1beta by ELISA.

FIG. 15. HLA-A02:01 restricted peptides in complex with MEL5/MEL8 TCRs, One crystal was used for determining each structure, *=figures in brackets refer to outer resolution shell, where applicable, ¹=Figures in brackets are rms targets, ²==Coordinate Estimated Standard Uncertainty in (Å), calculated based on maximum likelihood statistics.

FIG. 16. Supplementary 3D structures. (A) Melan A26-35 (EAAGIGILTV) peptide shown as sticks, when bound to MEL8, top-down view of peptide presented by HLA A*02:01 (grey, shown as surface), MEL8 CDR loops (indicated) are shown as cartoon. Crossing angle is indicated by line.

FIG. 17. Contacts between the MEL8 TCR and HLA A*02:01-EAAGIGILTV

FIG. 18. Tetramer staining of MEL5 T-cell clone with HLA A2 Melan A, BST222-31 (LLLGIGILVL) or IMP-2367-376 (NLSALGIFST) tetramers. Gates were set on single lymphocytes and live CD3+CD8+ cells. Mean Fluorescence Intensities (MFIs) are shown for each stain. Irrelevant tetramer made with HLA A2-ILAKFLHWL (hTERT540-548).

FIG. 19. Peptide activation assay with MEL5 T-cell clone and Melan A, BST2 (9mer-LLLGIGILV and 10mer-LLLGIGILVL) and IMP2 peptides (1×10-7 M). Overnight incubation and MIP-1B ELISA. Mean and standard error from duplicate samples is shown.

FIG. 20. MEL5 T-cell clone recognition of MOLT3 cell line transduced with HLA A2, and genes for Melan A, BST2 or IMP2. Untransduced (−ve) and Collagen gene transduced MOLT3s used as negative controls. Overnight incubation and TNF ELISA. Mean and standard experimental error from duplicate samples is shown.

FIG. 21. Contacts between the MEL5 TCR and HLA A*02:01-LLLGIGILVL

FIG. 22. Contacts between the MEL5 TCR and HLA A*02:01-NLSALGIFST

FIG. 23. Theoretical Contacts between the ST8 TCR and HLA-A2-LLLGIGILVL as determined by homology modelling

FIG. 24. Theoretical Contacts between the MEL8 TCR and HLA-A2-NLSALGIFST as determined by homology modelling

FIG. 25. Overnight activation of MEL8 T cell clone co-incubated with peptides and HLA A2+T2 antigen presenting cells was assessed by MIP-1beta ELISA. Melan-A26-35 (EAAGIGILTV), BST2222-31 (LLLGIGILVL) and IMP2367-376 (NLSALGIFST) peptides were used alone or combined as shown in the boxes below the X-axis and at the indicated concentrations. Mean and standard error of duplicate conditions is shown.

FIG. 26. Overnight activation of DMF4 or MEL5 TCR transduced and untransduced (−ve) CD8+ T-cells to Melan A, BST2 and IMP2 peptides, assessed by MIP-1beta ELISA. Mean and standard error of duplicate conditions is shown.

FIG. 27. Reactivity of DMF4 and MEL5 TCR transduced T-cells to Melan A peptide was assessed by a MIP-1beta ELISA following overnight activation. Mean and standard error of duplicate conditions is shown.

FIG. 28. Reactivity of DMF4 or MEL5 TCR transduced, and un-transduced (−ve) CD8+ T-cells to HLA A2+ melanoma cell lines; MEL624, MM909.24 and MEL526, assessed by TAPI-0 assay (4.5 h). Reactivity is expressed as the percentage of CD8+/rCD2+ cells producing TNF (rCD2 used as a TCR co-marker).

FIG. 29. MEL8 TCR transduced and un-transduced CD8 T-cells tested against cancer cell lines+/−HLA A2. Background reactivity of the T-cells towards HLA A2neg cancer cell lines has been subtracted from the reactivity seen with HLA A2+ cells. Reactivity is expressed as the percentage of TNF+/CD107a+ of CD8+/rCD2+ cells. PC-3 and MIA PaCa-2 transduced with HLA A2. Autologous MM909.24, MD-MB-231 and MONO-MAC-1 are Beta-2 microglobulin KO and HLA A2 transduced as single chain construct with Beta-2 microglobulin.

FIG. 30a Staining of TIL from melanoma patient MM909.24 using HLA A2 Melan A, BST2 and IMP2 tetramers. FIG. 30b. Purified CD8+ T cells from four HLA A2 donors were primed with Melan-A26-35 (EAAGIGILTV), BST222-31 (LLLGIGILVL) or IMP2366-376 (NLSALGIFST) peptides for 4 weeks, then stained with Melan A, BST2 and IMP2 tetramers. Irrelevant tetramer made with ILAKFLHWL (hTERT540-548).

FIG. 31. Summary Venn diagram of alpha (TRA) and beta (TRB) TCR chains from Melan A, BST2 and IMP2 tetramer sorts.

FIG. 32. TCR alpha and TCR beta chains from sequencing of Melan-A26-35 (EAAGIGILTV) tetramer sorted TILs from patient MM909.24. Shared TCR sequences with BST2 or IMP2 tetramers are indicated by hatched BST222-31 (LLLGIGILVL) or grey IMP2367-376 (NLSALGIFST) squares. TCR sequences shared between all three sorts are indicated with asterisks. The TCR of clone MEL8 is indicated with a triangle.

FIG. 33. TCR alpha and TCR beta chains from sequencing of BST222-31 (LLLGIGILVL) tetramer sorted TILs from patient MM909.24. Shared TCR sequences with Melan-A26-35 (EAAGIGILTV) or IMP2367-376 (NLSALGIFST) tetramers are indicated by black (Melan A) or grey (IMP2) squares. TCR sequences shared between all three sorts are indicated with asterisks. The TCR of clone MEL8 is indicated with a triangle.

FIG. 34. TCR alpha and TCR beta chains from sequencing of IMP2367-376 (NLSALGIFST) tetramer sorted TILs from patient MM909.24. Shared TCR sequences with Melan-A26-35 (EAAGIGILTV) or BST222-31 (LLLGIGILVL) tetramers are indicated by black (Melan A) or hatched (BST2) squares. TCR sequences shared between all three sorts are indicated with asterisks. The TCR of clone MEL8 is indicated with a triangle.

FIG. 35. [Upper] Flow based killing assay of the IMP2/Melan A T-cell line versus primary autologous AML cancer cells vs CD8+ T-cells from AML patient ME91-[Lower] flow based killing assay of the IMP2/Melan A T-cell line versus primary autologous CLL cancer cells vs CD8+ T-cells from CLL patient U386. Mean and SEM of duplicate conditions are shown. Gates set on single lymphocytes and live CD8+CD3+ cells. The percentage of tetramer+ T cells is shown for each gate. HLA A2-ALWGPDPAAA (preproinsulin 15-24) used as an irrelevant tetramer.

FIG. 36. Staining of CLL9 with Melan-A26-35 (EAAGIGILTV), BST222-31 (LLLGIGILVL) and IMP-2367-376 (NLSALGIFST) tetramers. HLA A2-ALWGPDPAAA (preproinsulin 15-24)=irrelevant tetramer. Mean fluorescence of staining (MFI).

FIG. 37. Overnight activation of CLL9 with peptides followed by MIP-1beta ELISA.

FIG. 38. Flow based overnight killing assay with CLL9 and primary autologous CLL cancer cells. CFSE labelled reference cells used to enumerate the CLL cancer cell survival+/−T-cells.

FIG. 39. Flow based overnight killing assay with CLL9 versus cancer lines, C1Rs and healthy cells. For the healthy cells CFSE labelled reference cells were used to enumerate the healthy cells+/−incubation with T-cells.

FIG. 40. Overnight activation assay with clone CLL9 (Upper) and MEL5 (Lower) versus melanomas, C1Rs and healthy cells followed by a TNF ELISA. Cancer cells+/−HLA A2 (either transgene or CRISPR/cas9 knock-out), HLA A2+ healthy cell lines (i.e. normal cells) and whole PBMCs or blood derived cell subset from HLA A2+ and HLA A2neg donors. For MEL5 the healthy cells were pulsed with 10-5 M EAAGIGILTV peptide then washed before assay, to act as a positive control for cell line recognition. Error bars depict standard deviation of duplicate conditions.

### DEFINITIONS

Suitably, the polypeptides and polynucleotides used in the present invention are isolated. An “isolated” polypeptide or polynucleotide is one that is removed from its original environment. For example, a naturally occurring polypeptide or polynucleotide is isolated if it is separated from some or all of the coexisting materials in the natural system. A polynucleotide is considered to be isolated if, for example, it is cloned into a vector that is not a part of its natural environment.

“Naturally occurring” or “native”, which terms are interchangeable, when used with reference to a polypeptide or polynucleotide sequence means a sequence found in nature and not synthetically modified.

The term “artificial” when used with reference to a polypeptide or polynucleotide sequence means a sequence not found in nature which is, for example, a synthetic modification of a natural sequence, or contains an unnatural polypeptide or polynucleotide sequence.

The term “engineered” when used with reference to a cell means a cell not found in nature which is, for example, a synthetic modification of a natural cell, for example, because it contains or expresses foreign elements and/or lacks natural elements.

The term “heterologous” when used with reference to the relationship of one polynucleotide or polypeptide to another polynucleotide or polypeptide indicates that the two or more sequences are not found in the same relationship to each other in nature.

The term “heterologous” when used with reference to the relationship of one polynucleotide or polypeptide sequence to a cell means a sequence which is not isolated from, derived from, expressed by said cell or associated with or based upon a naturally occurring polynucleotide or polypeptide sequence found in the said cell.

### DETAILED DESCRIPTION OF THE INVENTION

According to the invention there is provided an isolated anti-cancer T-cell receptor (TCR), or anti-cancer binding fragment thereof, that binds to a plurality of cancer polypeptide antigens or antigenic peptide fragments thereof wherein said cancer polypeptide antigens or antigenic peptide fragments thereof are distinct from each other and are presented at the cell surface of one or more than one type of cancer cell.

### TCR Binding

Preferably, the isolated TCR or anti-cancer binding fragment thereof of the invention binds or specifically binds to the cancer polypeptide antigen or antigenic peptide fragment presented at the cell surface of the tumour cell or cancer cell optionally in complex with a peptide presenting molecule for example major histocompatibility complex (MHC) or an HLA, optionally class I or II, for example with HLA-A2, preferably selected from any of HLA-A*02, HLA-A*02:01, HLA-A*02:02, HLA-A*02:03, HLA-A*02:04, HLA-A*02:05, HLA-A*02:06, or HLA-A*02:07, alternatively MR1. Optionally, the binding or specific binding is independent of presentation of the cancer polypeptide antigen or antigenic peptide fragment as a complex with a peptide-presenting molecule, for example where the cancer polypeptide antigen or antigenic peptide fragment is expressed directly at the surface of the tumour cell or cancer cell.

As used herein, the term “bind” or “specifically binding” in relation to the binding of A to B means that A binds to B, for example at or within a respective specific binding site, domain or pocket, with an affinity typically associated with the binding of ligands to receptors or typically associated with molecules of the immune system, such as antibodies and T-cell receptors, optionally a binding affinity level of micromolar or nanomolar affinity, such that the affinity of binding of A to B exceeds or greatly exceeds that of the binding of A to other molecules not intended to be targeted by A, for example a non-cancer polypeptide antigen or antigenic peptide fragment presented at the cell surface of a non-tumour cell or non-cancer cell, optionally in the context of a complex with peptide presenting molecule as herein described, or for example a non-target cancer polypeptide antigen or antigenic peptide fragment thereof presented at the cell surface of a tumour cell, optionally in the context of a complex with peptide presenting molecule, which is not bound or recognised by the subject TCR. The term “being specifically bound” is to be interpreted in a similar sense.

Preferably, the isolated TCR or anti-cancer binding fragment thereof of the invention also “specifically recognises” the cancer polypeptide antigen or antigenic peptide fragments presented at the cell surface of a tumour cell or a cancer cell in that it both binds or specifically binds and is specifically reactive to said tumour cell or cancer cell and/or tumour cell or cancer cell presented cancer polypeptide antigen or antigenic peptide fragments, optionally in the context of a peptide presenting molecule. As used herein, the term “specifically reactive” when used in relation to the binding of A to B means that A is activated when bound to B, to an extent that greatly exceeds that of the binding of A to other molecules not intended to be targeted by A. For example, the TCR or T-cell comprising the TCR, is activated when bound to the cancer polypeptide antigen or antigenic peptide fragment presented tumour cell or cancer cell. The determination as to whether the TCR specifically recognises, i.e. if it specifically binds to and/or is specifically reactive, may be discovered by determining if upon interaction with the cancer polypeptide antigen or antigenic peptide fragment presented by the tumour or cancer cell, for example in the context of a peptide presenting molecule, the TCR, which is an immune cell presented receptor, induces the activation of the immune cell which presents the receptor or TCR (or in the case of a soluble receptor TCR induces an immune reaction) to a higher level than the activation measured in the absence of the tumour or cancer cell, for example a non-cancer polypeptide antigen or antigenic peptide fragment presented at the cell surface of a non-tumour cell or non-cancer cell optionally in the context of a complex with peptide presenting molecule as herein described or for example a non-target cancer polypeptide antigen or antigenic peptide fragment thereof presented at the cell surface of a tumour cell, optionally in the context of a complex with peptide presenting molecule as herein described which is not bound or recognised by the subject TCR. For example, such immune cell or T cell activation can be evaluated with any of the following measurements: cytokine release (IFN gamma or TNF alpha), chemokine release, immune cell proliferation, immune cell expression of activation markers, immune cell target cell killing, induction of transcription factors or of reporter genes of the immune cell.

### Cancer Polypeptide Antigen or Antigenic Peptide Fragment

The isolated TCR or binding fragment of the invention is an anti-cancer T-cell receptor (TCR), or anti-cancer binding fragment thereof, meaning that its specifically bind or and/or recognises cancer target cells or tissues, e.g. cancer cells or tissue for example which present on their surface cancer associated antigens (herein cancer polypeptide antigen or antigenic peptide fragment) As used herein, the terms “cancer,” “neoplasm,” and “tumour” are used interchangeably and, in either the singular or plural form, refer to cells that have undergone a malignant transformation that makes them pathological to the host organism.

Examples of such cancer polypeptide antigen or antigenic peptide fragment are known cancer antigens, for example these may be a cancer-testis antigen, such as NY-ESO-1, MART-1 (melanoma antigen recognized by T cells), WT1 (Wilms tumor 1), gp100 (glycoprotein 100), tyrosinase, PRAME (preferentially expressed antigen in melanoma), p53, HPV-E6/HPV-E7 (human papillomavirus), HBV, TRAIL, DR4, Thyroglobin, TGFBII frameshift antigen, LAGE-1A, KRAS, CMV (cytomegalovirus), CEA (carcinoembryonic antigen), AFP (α-fetoprotein), MAGE-A1, MAGE-A2, MAGE-A3, MAGE-A4, MAGE-A6, MAGE-A8, and MAGE-A9, MAGE-A10, or MAGE-A12.

Preferably the isolated anti-cancer T-cell receptor (TCR), or anti-cancer binding fragment thereof according to the invention binds, specifically binds and/or specifically recognises a cancer polypeptide antigen(s) or antigenic peptide fragment(s) thereof wherein, the cancer polypeptide antigen or antigenic peptide fragment comprises the amino acid sequence motif X-A/G-I/L-G-I-X wherein X is any amino acid, and wherein said isolated anti-cancer T-cell receptor (TCR), or anti-cancer binding fragment thereof binds to an amino acid sequence comprising said sequence motif. The terms A/G, I/L, indicate that at the indicated position within the sequence the amino acids A/G are equivalently substitutable, similarly for I/L, for example substitutable without loss of ability of the TCR or binding fragment to bind and/or recognise the sequence.

Preferably the cancer polypeptide antigen or antigenic peptide fragment thereof comprises an amino acid sequence motif selected from any of;

(a).
X-X-X-A/G-I/L-G-I-X-X-X
(b).
X-X-X-A/G-I/L-G-I-X-X
(c).
X-X-X-A/G-I/L-G-I-X
(d).
X-X-A/G-I/L-G-I-X-X-X
(e).
X-X-A/G-I/L-G-I-X-X
(f).
X-A/G-I/L-G-I-X-X-X
(h).
X-A/G-I/L-G-I-X-X,
or
(i).
X-A/G-I/L-G-I-X;

preferably wherein X is any amino acid, and preferably the isolated anti-cancer T-cell receptor (TCR), or anti-cancer binding fragment thereof according to the invention binds to an amino acid sequence comprising said sequence motif. The sequence motif may be at any position comprised within the cancer polypeptide antigen or antigenic peptide fragment and or may comprise or consist of the antigenic peptide fragment.

Preferably, the cancer polypeptide antigen is selected from any one or more of Melan-A polypeptide (SEQ ID NO: 72), BST2 polypeptide (SEQ ID NO: 73) and IMP2 polypeptide (SEQ ID NO: 74), or antigenic peptide fragment thereof.

Accordingly, isolated anti-cancer T-cell receptor (TCR), or anti-cancer binding fragment thereof of the invention, binds, specifically binds and/or specifically recognises a cancer polypeptide antigen(s) or antigenic peptide fragment(s) thereof selected from any of;
(a). Melan-A polypeptide (SEQ ID NO: 72), BST2 polypeptide (SEQ ID NO: 73) and IMP2 polypeptide (SEQ ID NO: 74),
(b). Melan-A polypeptide (SEQ ID NO: 72), and BST2 polypeptide (SEQ ID NO: 73,
(c). Melan-A polypeptide (SEQ ID NO: 72) and IMP2 polypeptide (SEQ ID NO: 74), or
(d). BST2 polypeptide (SEQ ID NO: 73) and IMP2 polypeptide (SEQ ID NO: 74); or according to any of (a) to (d) above an antigenic peptide fragment thereof respectively.

For example according to the invention the antigenic peptide fragment may comprise any of 8, 9, 10, 11, 12 consecutive amino acid residues of any one of the aforementioned cancer polypeptide antigens, preferably wherein the cancer polypeptide antigen is selected from any one or more of Melan-A polypeptide (SEQ ID NO: 72), BST2 polypeptide (SEQ ID NO: 73) and IMP2 polypeptide (SEQ ID NO: 74).

Preferably, the antigenic peptide fragments are selected from any one or more of the sequences; (a) EAAGIGILTV (SEQ ID NO: 75, Melan A antigenic peptide fragment), (b) LLLGIGILVL (SEQ ID NO: 76, BST2 antigenic peptide fragment), (c) NLSALGIFST (SEQ ID NO: 77, IMP2 antigenic peptide fragment).

Accordingly the isolated anti-cancer T-cell receptor (TCR), or anti-cancer binding fragment thereof according to the invention binds, specifically binds and/or recognises a plurality of cancer polypeptide antigens or antigenic peptide fragments thereof selected from any of;

(a).
(SEQ ID NO: 75)
EAAGIGILTV,
(SEQ ID NO: 76)
LLLGIGILVL
and
(SEQ ID NO: 77)
NLSALGIFST,
(b).
(SEQ ID NO: 75)
EAAGIGILTV
and
(SEQ ID NO: 76)
LLLGIGILVL,
(c).
(SEQ ID NO: 75)
EAAGIGILTV
and
(SEQ ID NO: 77)
NLSALGIFST,
or
(d).
(SEQ ID NO: 76)
LLLGIGILVL
and
(SEQ ID NO: 77)
NLSALGIFST.

According to the present invention the antigenic peptide fragment is presented at a cell surface by human leukocyte antigen (HLA) class I molecule, preferably wherein the cell is a cancer or tumour cell.

### TCR and Binding Fragment

A TCR of this invention comprises an α chain and a β chain. The extracellular region of each chain comprises three CDRs (CDR1, CDR2, CDR3) and four framework regions which are either side of the CDRs, and a constant region. The soluble form of the TCR consists of the extracellular domain (also called the antigen-binding domain). The Constant region is proximal to the cell membrane, followed by a transmembrane region and a short cytoplasmic tail, while the Variable region binds to the polypeptide antigen ligand. Each chain has a connecting peptide region which links the transmembrane and intracellular regions to the extracellular domain at its C terminus. An immature form of an entire TCR also comprises a leader peptide sequence at its N terminus which is removed after translation by cellular peptidases such as signal peptidase. A soluble TCR of the present invention lacks the C terminus transmembrane and intracellular domains.

Accordingly, a TCR to which this invention relates is a disulfide-linked membrane-anchored heterodimeric protein consisting of the highly variable alpha (α) and beta (β) chains that associate with the invariant CD3 chain molecules to form a complete functioning TCR. T-cells expressing this receptor are referred to as α:β (or αβ) T-cells. The ligand for most αβ T-cells is a peptide bound to an HLA molecule.

The variable domain is formed of variable regions of both the TCR α-chain and β-chain each of which has three hypervariable regions called complementarity determining regions (CDRs). There is also an additional area of variability on the β-chain (HV4) that does not normally contact antigen and, therefore, is not considered a CDR. In general, the antigen-binding site is formed by the CDR loops of the TCR α-chain and β-chain. CDR1α and CDR2α are encoded by the individual Vα genes whereas CDR1β and CDR2β are encoded by the individual Vβ genes. The CDR3 of the TCR α-chain is especially hypervariable due to the potential for nucleotide addition and removal around the joining of the variable (V) region and a joining (J) region. The TCR β-chain CDR3 has even more capacity for variation as it can also include a diversity (D) gene.

CDR3 is the main CDR responsible for recognizing HLA-bound antigenic peptides (the usual target for αβ TCRs). However, in some cases CDR1 of the α chain has also been shown to interact with the N-terminal part of the HLA-bound antigenic peptide, and CDR1 of the β-chain may interact with the C-terminal part of the HLA-bound antigenic peptide.

The TCR anti-cancer binding fragment of the invention can comprise or consist of a TCR the extracellular domain (also called the antigen-binding domain), e.g. a soluble form of the TCR, wherein the TCR comprises a TCR alpha chain variable domain and a TCR beta chain variable domain, such domains may be incorporated into bispecific CARs or TCRs. Alternatively, the binding fragment can comprise of a single-chain format in which TCR V domains are connected by a flexible peptide linker, so called scTCRs or may comprise of a single-variable-domain TCR (svd TCR) that utilizes only the variable domain of the β chain (Vβ). The scTCRs and Vβ fragments can be comprised within in TCR and CAR formats, but also can be used to create single-chain bispecific CARs and TCRs.

The term “domain”, when used with reference to a TCR, is generally used to refer to a part of the TCR formed of the corresponding region of the two chains. For example, the transmembrane regions of the α and β chains form the transmembrane domain.

The term “intracellular” domain or region is used interchangeably with the term “cytoplasmic” domain or region and in the literature this is sometimes referred to as the “cytosolic” domain or region.

The invention provides fragments of the TCR of the invention are provided which retain the function of cancer-specific binding. Suitably, fragments of the invention, such as anti-cancer binding fragments, substantially retain the ability of the TCR or fragment thereof to bind to its target antigen or epitope, for example to the binding motif of the invention presented on the surface of the cancer or tumour, for example the binding affinity of the fragment is 75% or more e.g. 80% or more e.g. 85% or more e.g. 90% or more e.g. 95% or more e.g. 98% or more e.g. 99% or more of that of the respective TCR that in soluble form has the TCR α and β chains.

According to a second aspect of the present invention provides an isolated anti-cancer T-cell receptor (TCR) or anti-cancer binding fragment thereof, wherein the TCR or binding fragment comprises:
(i) an alpha chain variable region comprising a complementarity-determining region (CDR3 alpha) comprising or consisting of any one of:

(SEQ ID NO: 1)
CAVQKLVF,
(SEQ ID NO: 2)
CAAAGGGADGLTF,
(SEQ ID NO: 3)
CAVNNARLMF,
(SEQ ID NO: 4)
CAVNTGGFKTIF,
(SEQ ID NO: 5)
CAAGGGADGLTF,
(SEQ ID NO: 6)
CALGFGNVLHC,
(SEQ ID NO: 116)
CAVNVAGKSTF
(SEQ ID NO: 117)
CAVGAGGFKTIF
(SEQ ID NO: 118)
CAVNYEDDKIIF,
or
(SEQ ID NO: 120)
CGSNTGNQFYF,

or a variant CDR that has 1, 2, 3, 4 or 5 amino acid variations selected from additions, substitutions and deletions with respect thereto; and/or
(ii) a beta chain variable region comprising a complementarity-determining region (CDR3 beta) comprising or consisting of any one of:

(SEQ ID NO: 26)
CASSPYSGSGETQYF,
(SEQ ID NO: 27)
CASSFAGTDTQYF,
(SEQ ID NO: 28)
CASSYSFTEATYEQYF,
(SEQ ID NO: 29)
CASSLTGLGQPQHF,
(SEQ ID NO: 30)
CASSYRGLGQPQHF,
(SEQ ID NO: 31)
CASSATVLGTEAFF,
(SEQ ID NO: 32)
CSVEESSGIYEQYF,
(SEQ ID NO: 33)
CASSSAGMGQPQHF,
(SEQ ID NO: 34)
CAWSSQGLGQPQHF,
(SEQ ID NO: 35)
CASTLGTGDGYTF,
(SEQ ID NO: 36)
CSAPQTGLGQPQHF,
(SEQ ID NO: 37)
CASSFGPLANYGYTF,
(SEQ ID NO: 38)
CASSQGLAGSNEQFF,
(SEQ ID NO: 39)
CSATGLAGLGEQFF,
(SEQ ID NO: 40)
CASSLTGLGQPQHF,
(SEQ ID NO: 41)
CASSPGTIYQPQHF,
(SEQ ID NO: 42)
CASAGGALVGYGYTF
(SEQ ID NO: 121)
CAWSETGLGTGELFF,
(SEQ ID NO: 122)
CASSESPLSGNEQYF,
(SEQ ID NO: 123)
CASSVGVQGSWEQYF,
or
(SEQ ID NO: 124)
CASSQGVLVIAGVGEQYF;

or a variant CDR that has 1, 2, 3, 4 or 5 amino acid variations selected from additions, substitutions and deletions with respect thereto, wherein the TCR or binding fragment binds to the cancer polypeptide antigens Melan-A polypeptide (SEQ ID NO: 72), BST2 polypeptide (SEQ ID NO: 73) and IMP2 polypeptide (SEQ ID NO: 74) or antigenic peptide fragments thereof, for example antigenic peptide fragments comprising or consisting of EAAGIGILTV (SEQ ID NO: 75), LLLGIGILVL (SEQ ID NO: 76) and NLSALGIFST (SEQ ID NO: 77).

According to the second aspect, the isolated anti-cancer T-cell receptor (TCR) or anti-cancer binding fragment thereof may further comprise:
(i) an alpha chain variable region further comprising a complementarity-determining region (CDR1 alpha) comprising the sequence DRGSQS (SEQ ID NO: 82) and a complementarity-determining region (CDR2 alpha) comprising the sequence IYSNGD (SEQ ID NO: 83), or a variant CDR respectively that has 1, 2, or 3 amino acid variations selected from additions, substitutions and deletions with respect thereto; and/or
(ii) a beta chain variable region comprising any one of:
(a). a CDR3 beta sequence comprising the sequence CASSPYSGSGETQYF (SEQ ID NO: 26), and CDR1 beta and CDR2 beta sequence comprising the CDR1 beta and CDR2 beta sequences respectively of TRBV6-5,
(b). a CDR3 beta sequence comprising the sequence CASSFAGTDTQYF (SEQ ID NO: 27), and CDR1 beta and CDR2 beta sequence comprising the CDR1 beta and CDR2 beta sequences respectively of TRBV27,
(c). a CDR3 beta sequence comprising the sequence CASSYSFTEATYEQYF (SEQ ID NO: 28), and CDR1 beta and CDR2 beta sequence comprising the CDR1 beta and CDR2 beta sequences respectively of TRBV6-5,
(d). a CDR3 beta sequence comprising the sequence CASSLTGLGQPQHF (SEQ ID NO: 29), and CDR1 beta and CDR2 beta sequence comprising the CDR1 beta and CDR2 beta sequences respectively of TRBV28,
(e). a CDR3 beta sequence comprising the sequence CASSYRGLGQPQHF (SEQ ID NO: 30), and CDR1 beta and CDR2 beta sequence comprising the CDR1 beta and CDR2 beta sequences respectively of TRBV28,
(f). a CDR3 beta sequence comprising the sequence CASSATVLGTEAFF (SEQ ID NO: 31), and CDR1 beta and CDR2 beta sequence comprising the CDR1 beta and CDR2 beta sequences respectively of TRBV19,
(g). a CDR3 beta sequence comprising the sequence CSVEESSGIYEQYF (SEQ ID NO: 32), and CDR1 beta and CDR2 beta sequence comprising the CDR1 beta and CDR2 beta sequences respectively of TRBV29-1,
(h). a CDR3 beta sequence comprising the sequence CASSSAGMGQPQHF (SEQ ID NO: 33), and CDR1 beta and CDR2 beta sequence comprising the CDR1 beta and CDR2 beta sequences respectively of TRBV27,
(i). a CDR3 beta sequence comprising the sequence CAWSSQGLGQPQHF (SEQ ID NO: 34), and CDR1 beta and CDR2 beta sequence comprising the CDR1 beta and CDR2 beta sequences respectively of TRBV30,
(j). a CDR3 beta sequence comprising the sequence CASTLGTGDGYTF (SEQ ID NO: 35), and CDR1 beta and CDR2 beta sequence comprising the CDR1 beta and CDR2 beta sequences respectively of TRBV19,
(k). a CDR3 beta sequence comprising the sequence CSAPQTGLGQPQHF (SEQ ID NO: 36), and CDR1 beta and CDR2 beta sequence comprising the CDR1 beta and CDR2 beta sequences respectively of TRBV20-1,
(l). a CDR3 beta sequence comprising the sequence CASSFGPLANYGYTF (SEQ ID NO: 37), and CDR1 beta and CDR2 beta sequence comprising the CDR1 beta and CDR2 beta sequences respectively of TRBV19,
(m). a CDR3 beta sequence comprising the sequence CASSQGLAGSNEQFF (SEQ ID NO: 38), and CDR1 beta and CDR2 beta sequence comprising the CDR1 beta and CDR2 beta sequences respectively of TRBV4-2,
(n). a CDR3 beta sequence comprising the sequence CSATGLAGLGEQFF (SEQ ID NO: 39), and CDR1 beta and CDR2 beta sequence comprising the CDR1 beta and CDR2 beta sequences respectively of TRBV20-1,
(o). a CDR3 beta sequence comprising the sequence CASSLTGLGQPQHF (SEQ ID NO: 40), and CDR1 beta and CDR2 beta sequence comprising the CDR1 beta and CDR2 beta sequences respectively of TRBV5-6,
(p). a CDR3 beta sequence comprising the sequence CASSPGTIYQPQHF (SEQ ID NO: 41), and CDR1 beta and CDR2 beta sequence comprising the CDR1 beta and CDR2 beta sequences respectively of TRBV4-3,
(q). a CDR3 beta sequence comprising the sequence CASAGGALVGYGYTF (SEQ ID NO: 42); and CDR1 beta and CDR2 beta sequence comprising the CDR1 beta and CDR2 beta sequences respectively of TRBV6-5,
(r). a CDR3 beta sequence comprising the sequence CAWSETGLGTGELFF (SEQ ID NO: 121) and CDR1 beta and CDR2 beta sequence comprising the CDR1 beta and CDR2 beta sequences respectively of TRBV30-02
(s). a CDR3 beta sequence comprising the sequence CASSESPLSGNEQYF (SEQ ID NO: 122), and CDR1 beta and CDR2 beta sequence comprising the CDR1 beta and CDR2 beta sequences respectively of TRBV2-02,
(t). a CDR3 beta sequence comprising the sequence CASSVGVQGSWEQYF (SEQ ID NO: 123), and CDR1 beta and CDR2 beta sequence comprising the CDR1 beta and CDR2 beta sequences respectively of TRBV9, or
(u). a CDR3 beta sequence comprising the sequence CASSQGVLVIAGVGEQYF (SEQ ID NO:124), and CDR1 beta and CDR2 beta sequence comprising the CDR1 beta and CDR2 beta sequences respectively of TRBV4-2; or, in any of (a) to (u), a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

According to the second aspect, the isolated anti-cancer T-cell receptor (TCR) or anti-cancer binding fragment thereof may comprise a beta chain variable region comprising any one of:
(a). a CDR3 beta sequence comprising the sequence CASSPYSGSGETQYF (SEQ ID NO: 26), and CDR1 beta and CDR2 beta sequences comprising the sequences MNHEY (SEQ ID NO:84) and SVGAGI (SEQ ID NO:97) respectively,
(b). a CDR3 beta sequence comprising the sequence CASSFAGTDTQYF (SEQ ID NO: 27), and CDR1 beta and CDR2 beta sequences comprising the sequences MNHEY (SEQ ID NO:84) and SMNVEV (SEQ ID NO:98) respectively,
(c). a CDR3 beta sequence comprising the sequence CASSYSFTEATYEQYF (SEQ ID NO: 28), and CDR1 beta and CDR2 beta sequences comprising the sequences MNHEY (SEQ ID NO:84) and SVGAGI (SEQ ID NO:97) respectively,
(d). a CDR3 beta sequence comprising the sequence CASSLTGLGQPQHF (SEQ ID NO: 29), and CDR1 beta and CDR2 beta sequences comprising the sequences MDHEN (SEQ ID NO:85) and SYDVKM (SEQ ID NO:99) respectively,
(e). a CDR3 beta sequence comprising the sequence CASSYRGLGQPQHF (SEQ ID NO: 30), and CDR1 beta and CDR2 beta sequences comprising the sequences MDHEN (SEQ ID NO:85) and SYDVKM (SEQ ID NO:99) respectively,
(f). a CDR3 beta sequence comprising the sequence CASSATVLGTEAFF (SEQ ID NO: 31), and CDR1 beta and CDR2 beta sequences comprising the sequences LNHDA (SEQ ID NO:86) and SQIVND (SEQ ID NO:100) respectively,
(g). a CDR3 beta sequence comprising the sequence CSVEESSGIYEQYF (SEQ ID NO: 32), and CDR1 beta and CDR2 beta sequences comprising the sequences SQVTM (SEQ ID NO:87) and ANQGSEA (SEQ ID NO:101) respectively,
(h). a CDR3 beta sequence comprising the sequence CASSSAGMGQPQHF (SEQ ID NO: 33), and CDR1 beta and CDR2 beta sequences comprising the sequences MNHEY (SEQ ID NO:84) and SMNVEV (SEQ ID NO:98) respectively,
(i). a CDR3 beta sequence comprising the sequence CAWSSQGLGQPQHF (SEQ ID NO: 34), and CDR1 beta and CDR2 beta sequences comprising the sequences GTSNPN (SEQ ID NO:88) and SVGIG (SEQ ID NO:102) respectively,
(j). a CDR3 beta sequence comprising the sequence CASTLGTGDGYTF (SEQ ID NO: 35), and CDR1 beta and CDR2 beta sequences comprising the sequences LNHDA (SEQ ID NO:86) and SQIVND (SEQ ID NO:100) respectively,
(k). a CDR3 beta sequence comprising the sequence CSAPQTGLGQPQHF (SEQ ID NO: 36), and CDR1 beta and CDR2 beta sequences comprising the sequences DFQATT (SEQ ID NO:89) and SNEGSKA (SEQ ID NO:103) respectively,
(l). a CDR3 beta sequence comprising the sequence CASSFGPLANYGYTF (SEQ ID NO: 37), and CDR1 beta and CDR2 beta sequences comprising the sequences LNHDA (SEQ ID NO:86) and SQIVND (SEQ ID NO:100) respectively,
(m). a CDR3 beta sequence comprising the sequence CASSQGLAGSNEQFF (SEQ ID NO: 38), and CDR1 beta and CDR2 beta sequences comprising the sequences LGHNA (SEQ ID NO:90) and YNFKEQ (SEQ ID NO:104) respectively,
(n). a CDR3 beta sequence comprising the sequence CSATGLAGLGEQFF (SEQ ID NO: 39), and CDR1 beta and CDR2 beta sequences comprising the sequences DFQATT (SEQ ID NO:89) and SNEGSKA (SEQ ID NO:103) respectively,
(o). a CDR3 beta sequence comprising the sequence CASSLTGLGQPQHF (SEQ ID NO: 40), and CDR1 beta and CDR2 beta sequences comprising the sequences SGHDT (SEQ ID NO:91) and YYEEEE (SEQ ID NO:105) respectively,
(p). a CDR3 beta sequence comprising the sequence CASSPGTIYQPQHF (SEQ ID NO: 41), and CDR1 beta and CDR2 beta sequences comprising the sequences LGHNA (SEQ ID NO:90) and YSLEER (SEQ ID NO:106) respectively, or
(q). a CDR3 beta sequence comprising the sequence CASAGGALVGYGYTF (SEQ ID NO: 42); and CDR1 beta and CDR2 beta sequences comprising the sequences MNHEY (SEQ ID NO:84) and SVGAGI (SEQ ID NO:97) respectively,
(r). a CDR3 beta sequence comprising the sequence, CAWSETGLGTGELFF (SEQ ID NO: 121), and CDR1 beta and CDR2 beta sequences comprising the sequences GTSNPN (SEQ ID NO:88) and SVGIG (SEQ ID NO:102), respectively,
(s). a CDR3 beta sequence comprising the sequence CASSESPLSGNEQYF (SEQ ID NO: 122), and CDR1 beta and CDR2 beta sequences comprising the sequences SNHLY (SEQ ID NO:127) and FYNNEI (SEQ ID NO:128), respectively,
(t). a CDR3 beta sequence comprising the sequence CASSVGVQGSWEQYF (SEQ ID NO: 123), and CDR1 beta and CDR2 beta sequence comprising the sequences SGDLS (SEQ ID NO:129) and HYYNGE (SEQ ID NO:130), respectively,
(u). a CDR3 beta sequence comprising the sequence CASSQGVLVIAGVGEQYF (SEQ ID NO:124), and CDR1 beta and CDR2 beta sequence comprising the sequences LGHNA (SEQ ID NO:90) and YNFKEQ (SEQ ID NO:104), respectively; or, in any f (a) to (u) above, a variant CDR3 beta that has 1, 2, 3, 4 or 5 amino acid variations selected from additions, substitutions and deletions with respect thereto, and/or a variant CDR1 beta or CDR2 beta sequence that has 1, 2, or 3 amino acid variations selected from additions, substitutions and deletions with respect thereto.

According to the second aspect, the isolated anti-cancer T-cell receptor (TCR) or anti-cancer binding fragment thereof preferably comprises any of:
(a) an alpha chain variable region comprising a CDR1 alpha comprising the sequence DRGSQS (SEQ ID NO:82), a CDR2 alpha comprising the sequence IYSNGD (SEQ ID NO:83), and a CDR3 alpha comprising the sequence CAVQKLVF (SEQ ID NO:1) and a beta chain variable region comprising a CDR1 beta comprising the sequence MNHEY (SEQ ID NO:84), a CDR2 beta comprising the sequence SVGAGI (SEQ ID NO:97), and a CDR3 beta comprising the sequence CASSYSFTEATYEQYF (SEQ ID NO:28);
(b) an alpha chain variable region comprising a CDR1 alpha comprising the sequence DRGSQS (SEQ ID NO:82), a CDR2 alpha comprising the sequence IYSNGD (SEQ ID NO:83), a CDR3 alpha comprising the sequence CAVGAGGFKTIF (SEQ ID NO:117), and a beta chain variable region comprising a CDR1 beta comprising the sequence SNHLY (SEQ ID NO:127), a CDR2 beta comprising the sequence FYNNEI (SEQ ID NO:128), a CDR3 beta comprising the sequence CASSESPLSGNEQYF (SEQ ID NO:122);
(c) an alpha chain variable region comprising a CDR1 alpha comprising the sequence DRGSQS (SEQ ID NO:82), a CDR2 alpha comprising the sequence IYSNGD (SEQ ID NO:83), a CDR3 alpha comprising the sequence CAVNYEDDKIIF (SEQ ID NO:118), and a beta chain variable region comprising a CDR1 beta comprising the sequence SGDLS (SEQ ID NO: 129), a CDR2 beta comprising the sequence HYYNGE (SEQ ID NO:130), a CDR3 beta comprising the sequence CASSWAGPVEQYF (SEQ ID NO:53);
(d) an alpha chain variable region comprising a CDR1 alpha comprising the sequence DRGSQS (SEQ ID NO:82), a CDR2 alpha comprising the sequence IYSNGD (SEQ ID NO:83), a CDR3 alpha comprising the sequence CGSNTGNQFYF (SEQ ID NO:120), and a beta chain variable region comprising a CDR1 beta comprising the sequence GTSNPN (SEQ ID NO:88), a CDR2 beta comprising the sequence SVGIG (SEQ ID NO: 102), a CDR3 beta comprising the sequence CAWSSQGLGQPQHF (SEQ ID NO:34); or
(e) an alpha chain variable region comprising a CDR1 alpha comprising the sequence DRGSQS (SEQ ID NO:82), a CDR2 alpha comprising the sequence IYSNGD (SEQ ID NO:83), a CDR3 alpha comprising the sequence CAVNNARLMF (SEQ ID NO:3), and a beta chain variable region comprising a CDR1 beta comprising the sequence LGHNA (SEQ ID NO:90), a CDR2 beta comprising the sequence YNFKEQ (SEQ ID NO:104), a CDR3 beta comprising the sequence CASSQGVLVIAGVGEQYF (SEQ ID NO:124).

According to a third aspect of the present invention there is provided an isolated anti-cancer T-cell receptor (TCR) or anti-cancer binding fragment thereof, wherein the TCR or binding fragment comprises:
(i) an alpha chain variable region comprising a complementarity-determining region (CDR3 alpha) comprising or consisting of any one of:

(SEQ ID NO: 7)
CAVNIGARLMF
(SEQ ID NO: 8)
CAVASDGQKLLF,
or
(SEQ ID NO: 9)
CAVYGGSQGNLIF;

or a variant CDR that has 1, 2, 3, 4 or 5 amino acid variations selected from additions, substitutions and deletions with respect thereto; and/or
(ii) a beta chain variable region comprising a complementarity-determining region (CDR3 beta) comprising or consisting of any one of:

(SEQ ID NO: 43)
CASSFQGLGQPQHF,
(SEQ ID NO: 44)
CASTLGGTLGSPLHF
or
(SEQ ID NO: 45)
CASTLGQGWEQYF;

or a variant CDR that has 1, 2, 3, 4 or 5 amino acid variations selected from additions, substitutions and deletions with respect thereto, preferably wherein the TCR or binding fragment binds to the cancer polypeptide antigens BST2 polypeptide (SEQ ID NO: 73) and IMP2 polypeptide (SEQ ID NO: 74) or antigenic peptide fragments thereof, for example antigenic peptide fragments comprising or consisting of LLLGIGILVL (SEQ ID NO: 76 BST2) and NLSALGIFST (SEQ ID NO: 77, IMP2).

According to the third aspect of the present invention there is provided an isolated anti-cancer T-cell receptor (TCR) or anti-cancer binding fragment thereof which further comprises:
(i) an alpha chain variable region further comprising a complementarity-determining region (CDR1 alpha) comprising the sequence DRGSQS (SEQ ID NO: 82) and a complementarity-determining region (CDR2 alpha) comprising the sequence IYSNGD (SEQ ID NO: 83), or a variant CDR that has 1, 2, or 3 amino acid variations selected from additions, substitutions, and deletions with respect thereto; and/or
(ii) a beta chain variable region comprising any one of:
(a). a CDR3 beta sequence comprising the sequence CASSFQGLGQPQHF (SEQ ID NO: 43) and CDR1 beta and CDR2 beta sequence comprising the CDR1 beta and CDR2 beta sequences respectively of TRBV28,
(b). a CDR3 beta sequence comprising the sequence CASTLGGTLGSPLHF (SEQ ID NO: 44) and CDR1 beta and CDR2 beta sequence comprising the CDR1 beta and CDR2 beta sequences respectively of TRBV19, or
(c). a CDR3 beta sequence comprising the sequence CASTLGQGWEQYF (SEQ ID NO: 45) and CDR1 beta and CDR2 beta sequence comprising the CDR1 beta and CDR2 beta sequences respectively of TRBV6-2; or in any of (a) to (c), a variant CDR3 beta that has 1, 2, 3, 4 or 5 amino acid variations selected from additions, substitutions and deletions with respect thereto, and/or a variant CDR1 beta or CDR2 beta sequence that has 1, 2, or 3 amino acid variations selected from additions, substitutions and deletions with respect thereto.

According to the third aspect of the present invention there is provided an isolated anti-cancer T-cell receptor (TCR) or anti-cancer binding fragment thereof which comprises a beta chain variable region comprising any one of:
(a). a CDR3 beta sequence comprising the sequence CASSFQGLGQPQHF (SEQ ID NO: 43) and CDR1 beta and CDR2 beta sequences comprising the sequences MDHEN (SEQ ID NO:85) and SYDVKM (SEQ ID NO:99) respectively,
(b). a CDR3 beta sequence comprising the sequence CASTLGGTLGSPLHF (SEQ ID NO: 44) and CDR1 beta and CDR2 beta sequences comprising the sequences LNHDA (SEQ ID NO:86) and SQIVND (SEQ ID NO:100) respectively, or
(c). a CDR3 beta sequence comprising the sequence CASTLGQGWEQYF (SEQ ID NO: 45) and CDR1 beta and CDR2 beta sequences comprising the sequences MNHEY (SEQ ID NO:84) and SVGEGT (SEQ ID NO: 107) respectively, or in any of (a) to (c), a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

According to a fourth aspect of the present invention there is provided an isolated anti-cancer T-cell receptor (TCR) or anti-cancer binding fragment thereof which comprises:
(i) an alpha chain variable region comprising a complementarity-determining region (CDR3 alpha) comprising or consisting of any one of:

(SEQ ID NO: 10)
CAVNQFYF,
(SEQ ID NO: 11)
CAGGGGADGLTF,
(SEQ ID NO: 12)
CAANNARLMF,
or
(SEQ ID NO: 13)
CAVNQGGKLIF;

or a variant CDR that has 1, 2, 3, 4 or 5 amino acid variations selected from additions, substitutions and deletions with respect thereto; and/or
(ii) a beta chain variable region comprising a complementarity-determining region (CDR3 beta) comprising or consisting of any one of:

(SEQ ID NO: 46)
CASSLQGLGQPQHF,
(SEQ ID NO: 47)
CASSLSVISTGELFF,
(SEQ ID NO: 48)
CASGKGQIFAGELFF,
(SEQ ID NO: 49)
CATSGLAGAYEQYF,
(SEQ ID NO: 50)
CASSEYTSGNQPQHF,
(SEQ ID NO: 51)
CASSPAGLGQPQHF,
(SEQ ID NO: 52)
CASSPDPFGLAGNEQFF,
or
(SEQ ID NO: 125)
CASSEAVLYVDTQYF,

or a variant CDR, respectively, that has 1, 2, 3, 4 or 5 amino acid variations selected from additions, substitutions, and deletions with respect thereto, wherein the TCR or binding fragment binds to the cancer polypeptide antigens Melan-A polypeptide (SEQ ID NO: 72) and BST2 polypeptide (SEQ ID NO: 73) or antigenic peptide fragments thereof, for example antigenic peptide fragments comprising or consisting of EAAGIGILTV (SEQ ID NO: 75, MelanA) and LLLGIGILVL (SEQ ID NO: 76, BST2).

According to the fourth aspect of the present invention there is provided an isolated anti-cancer T-cell receptor (TCR) or anti-cancer binding fragment thereof which comprises:
(i) an alpha chain variable region further comprising a complementarity-determining region (CDR1 alpha) comprising the sequence DRGSQS (SEQ ID NO: 82) and a complementarity-determining region (CDR2 alpha) comprising the sequence IYSNGD (SEQ ID NO: 83), or a variant CDR that has 1, 2, or 3 amino acid variations selected from additions, substitutions and deletions with respect thereto; and/or
(ii) a beta chain variable region comprising any one of:
(a). a CDR3 beta sequence comprising the sequence CASSLQGLGQPQHF (SEQ ID NO: 46) and CDR1 beta and CDR2 beta sequence comprising the CDR1 beta and CDR2 beta sequences respectively of TRBV28
(b). a CDR3 beta sequence comprising the sequence CASSLSVISTGELFF (SEQ ID NO: 47) and CDR1 beta and CDR2 beta sequence comprising the CDR1 beta and CDR2 beta sequences respectively of TRBV27
(c). a CDR3 beta sequence comprising the sequence CASGKGQIFAGELFF (SEQ ID NO: 48) and CDR1 beta and CDR2 beta sequence comprising the CDR1 beta and CDR2 beta sequences respectively of TRBV28
(d). a CDR3 beta sequence comprising the sequence CATSGLAGAYEQYF (SEQ ID NO: 49) and CDR1 beta and CDR2 beta sequence comprising the CDR1 beta and CDR2 beta sequences respectively of TRBV7-2
(e). a CDR3 beta sequence comprising the sequence CASSEYTSGNQPQHF (SEQ ID NO: 50) and CDR1 beta and CDR2 beta sequence comprising the CDR1 beta and CDR2 beta sequences respectively of TRBV6-1
(f). a CDR3 beta sequence comprising the sequence CASSPAGLGQPQHF (SEQ ID NO: 51) and CDR1 beta and CDR2 beta sequence comprising the CDR1 beta and CDR2 beta sequences respectively of TRBV6-2,
(g). a CDR3 beta sequence comprising the sequence CASSPDPFGLAGNEQFF (SEQ ID NO:52) and CDR1 beta and CDR2 beta sequence comprising the CDR1 beta and CDR2 beta sequences respectively of TRBV7-2, or
(h). a CDR3 beta sequence comprising the sequence CASSEAVLYVDTQYF (SEQ ID NO: 125) and CDR1 beta and CDR2 beta sequence comprising the CDR1 beta and CDR2 beta sequences respectively of TRBV6-1, or, in any of (a) to (h) a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

According to the fourth aspect the isolated anti-cancer T-cell receptor (TCR) or anti-cancer binding fragment thereof preferably comprises any of:
(a). a CDR3 beta sequence comprising the sequence CASSLQGLGQPQHF (SEQ ID NO: 46) and CDR1 beta and CDR2 beta sequences comprising the sequences MDHEN (SEQ ID NO:85) and SYDVKM (SEQ ID NO:99) respectively,
(b). a CDR3 beta sequence comprising the sequence CASSLSVISTGELFF (SEQ ID NO: 47) and CDR1 beta and CDR2 beta sequences comprising the sequences MNHEY (SEQ ID NO:84) and SMNVEV (SEQ ID NO:98) respectively,
(c). a CDR3 beta sequence comprising the sequence CASGKGQIFAGELFF (SEQ ID NO: 48) and CDR1 beta and CDR2 beta sequences comprising the sequences MDHEN (SEQ ID NO:85) and SYDVKM (SEQ ID NO:99) respectively,
(d). a CDR3 beta sequence comprising the sequence CATSGLAGAYEQYF (SEQ ID NO: 49) and CDR1 beta and CDR2 beta sequences comprising the sequences SGHTA (SEQ ID NO:92) and FQGNSA (SEQ ID NO:108) respectively,
(e). a CDR3 beta sequence comprising the sequence CASSEYTSGNQPQHF (SEQ ID NO: 50) and CDR1 beta and CDR2 beta sequences comprising the sequences MNHNS (SEQ ID NO:93) and SASEGT (SEQ ID NO:109) respectively,
(f). a CDR3 beta sequence comprising the sequence CASSPAGLGQPQHF (SEQ ID NO: 51) and CDR1 beta and CDR2 beta sequences comprising the sequences MNHEY (SEQ ID NO:84) and SVGEGT (SEQ ID NO:107) respectively, or
(g). a CDR3 beta sequence comprising the sequence CASSPDPFGLAGNEQFF (SEQ ID NO:52) and CDR1 beta and CDR2 beta sequences comprising the sequences SGHTA (SEQ ID NO:92) and FQGNSA (SEQ ID NO:108) respectively; or
(h). a CDR3 beta sequence comprising the sequence CASSEAVLYVDTQYF (SEQ ID NO: 125) and CDR1 beta and CDR2 beta sequences comprising the sequences MNHNS (SEQ ID NO:93) and SASEGT (SEQ ID NO:109) respectively, or, in any of (a) to (h), a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

According to the fourth aspect of the present invention there is provided an isolated anti-cancer T-cell receptor (TCR) or anti-cancer binding fragment, wherein the TCR or binding fragment comprises:
(a) an alpha chain variable region comprising a CDR1 alpha comprising the sequence DRGSQS (SEQ ID NO:82), a CDR2 alpha comprising the sequence IYSNGD (SEQ ID NO:83), and a CDR3 alpha comprising the sequence CAVNQGGKLIF (SEQ ID NO:13) and a beta chain variable region comprising a CDR1 beta comprising the sequence MNHNS (SEQ ID NO:93), a CDR2 beta comprising the sequence SASEGT (SEQ ID NO: 109), and a CDR3 beta comprising the sequence CASSEAVLYVDTQYF (SEQ ID NO: 125).

According to a fifth aspect of the present invention there is provided an isolated anti-cancer T-cell receptor (TCR) or anti-cancer binding fragment thereof, wherein the TCR or binding fragment comprises:
(i) an alpha chain variable region comprising a complementarity-determining region (CDR3 alpha) comprising or consisting of any one of:

(SEQ ID NO: 14)
CAVNSGGGADGLTF,
(SEQ ID NO: 15)
CAVSIGFGNVLHC,
(SEQ ID NO: 16)
CAVHTGGFKTIF,
(SEQ ID NO: 17)
CAVDNQGGKLIF,
or
(SEQ ID NO: 18)
CAVSIGFGNVLHC;

or a variant CDR that has 1, 2, 3, 4 or 5 amino acid variations selected from additions, substitutions and deletions with respect thereto; and/or
(ii) a beta chain variable region comprising a complementarity-determining region (CDR3 beta) comprising or consisting of any one of:

(SEQ ID NO: 53)
CASSWAGPVEQYF,
(SEQ ID NO: 54)
CASSLGLAGNEQYF,
(SEQ ID NO: 55)
CASSSGLTDTQYF,
(SEQ ID NO: 56)
CASSLGIISGQPQHF,
(SEQ ID NO: 57)
CASSLGGGGYEQYF,
(SEQ ID NO: 58)
CAISPGEGTDTQYF,
(SEQ ID NO: 59)
CASSYTGTLSYEQYF
(SEQ ID NO: 60)
CASSYVGLGSPLHF,
(SEQ ID NO: 61)
CATSRDMDPEQYF,
(SEQ ID NO: 62)
CASSLGGADTQYF,
(SEQ ID NO: 63)
CASSFLQGTGELFF,
(SEQ ID NO: 64)
CASRVGILSGELFF,
(SEQ ID NO: 65)
CATSRDRGWEQFF,
(SEQ ID NO: 66)
CASSLSGAEAFF,
(SEQ ID NO: 67)
CASSPSWAHETQYF,
(SEQ ID NO: 68)
CASSPSGTVYEKLFF,
(SEQ ID NO: 69)
CSASEGIGQPQHF,
(SEQ ID NO: 70)
CASSPGLTANVLTF,
(SEQ ID NO: 71)
CASSQEQLAGPEQYF,
or
(SEQ ID NO: 126)
CASSFAGTDTQYF,

or a variant CDR that has 1, 2, 3, 4 or 5 amino acid variations selected from additions, substitutions, and deletions with respect thereto, wherein the TCR or binding fragment binds to the cancer polypeptide antigens Melan-A polypeptide (SEQ ID NO: 72) and IMP2 polypeptide (SEQ ID NO: 74) or antigenic peptide fragments thereof, for example antigenic peptide fragments comprising or consisting of EAAGIGILTV (SEQ ID NO: 75, MelanA) and NLSALGIFST (SEQ ID NO: 77, IMP2).

According to the fifth aspect the isolated anti-cancer T-cell receptor (TCR) or anti-cancer binding fragment thereof further comprises:
(i) an alpha chain variable region further comprising a complementarity-determining region (CDR1 alpha) comprising the sequence DRGSQS (SEQ ID NO: 82) and a complementarity-determining region (CDR2 alpha) comprising the sequence IYSNGD (SEQ ID NO: 83), or a variant CDR that has 1, 2, or 3 amino acid variations selected from additions, substitutions and deletions with respect thereto; and/or
(ii) a beta chain variable region comprising any one of:
(a). a CDR3 beta sequence comprising the sequence CASSWAGPVEQYF (SEQ ID NO: 53) and CDR1 beta and CDR2 beta sequence comprising the CDR1 beta and CDR2 beta sequences respectively of TRBV12-4,
(b). a CDR3 beta sequence comprising the sequence CASSLGLAGNEQYF (SEQ ID NO: 54) and CDR1 beta and CDR2 beta sequence comprising the CDR1 beta and CDR2 beta sequences respectively of TRBV27,
(c). a CDR3 beta sequence comprising the sequence CASSSGLTDTQYF (SEQ ID NO: 55) TRBV7-3 and CDR1 beta and CDR2 beta sequence comprising the CDR1 beta and CDR2 beta sequences respectively of TRBV7-3,
(d). a CDR3 beta sequence comprising the sequence CASSLGIISGQPQHF (SEQ ID NO: 56) and CDR1 beta and CDR2 beta sequence comprising the CDR1 beta and CDR2 beta sequences respectively of TRBV13,
(e). a CDR3 beta sequence comprising the sequence CASSLGGGGYEQYF (SEQ ID NO: 57) and CDR1 beta and CDR2 beta sequence comprising the CDR1 beta and CDR2 beta sequences respectively of TRBV27,
(f). a CDR3 beta sequence comprising the sequence CAISPGEGTDTQYF (SEQ ID NO: 58) and CDR1 beta and CDR2 beta sequence comprising the CDR1 beta and CDR2 beta sequences respectively of TRBV10-3,
(g). a CDR3 beta sequence comprising the sequence CASSYTGTLSYEQYF (SEQ ID NO: 59) and CDR1 beta and CDR2 beta sequence comprising the CDR1 beta and CDR2 beta sequences respectively of TRBV28,
(h). a CDR3 beta sequence comprising the sequence CASSYVGLGSPLHF (SEQ ID NO: 60) and CDR1 beta and CDR2 beta sequence comprising the CDR1 beta and CDR2 beta sequences respectively of TRBV6-5,
(i). a CDR3 beta sequence comprising the sequence CATSRDMDPEQYF (SEQ ID NO: 61) and CDR1 beta and CDR2 beta sequence comprising the CDR1 beta and CDR2 beta sequences respectively of TRBV15,
(j). a CDR3 beta sequence comprising the sequence CASSLGGADTQYF (SEQ ID NO: 62) and CDR1 beta and CDR2 beta sequence comprising the CDR1 beta and CDR2 beta sequences respectively of TRBV27,
(k). a CDR3 beta sequence comprising the sequence CASSFLQGTGELFF (SEQ ID NO: 63) and CDR1 beta and CDR2 beta sequence comprising the CDR1 beta and CDR2 beta sequences respectively of TRBV27,
(l). a CDR3 beta sequence comprising the sequence CASRVGILSGELFF (SEQ ID NO: 64) and CDR1 beta and CDR2 beta sequence comprising the CDR1 beta and CDR2 beta sequences respectively of TRBV27,
(m). a CDR3 beta sequence comprising the sequence CATSRDRGWEQFF (SEQ ID NO: 65) and CDR1 beta and CDR2 beta sequence comprising the CDR1 beta and CDR2 beta sequences respectively of TRBV15,
(n). a CDR3 beta sequence comprising the sequence CASSLSGAEAFF (SEQ ID NO: 66) and CDR1 beta and CDR2 beta sequence comprising the CDR1 beta and CDR2 beta sequences respectively of TRBV27,
(o). a CDR3 beta sequence comprising the sequence CASSPSWAHETQYF (SEQ ID NO: 67) and CDR1 beta and CDR2 beta sequence comprising the CDR1 beta and CDR2 beta sequences respectively of TRBV27,
(p). a CDR3 beta sequence comprising the sequence CASSPSGTVYEKLFF (SEQ ID NO: 68) and CDR1 beta and CDR2 beta sequence comprising the CDR1 beta and CDR2 beta sequences respectively of TRBV28,
(q). a CDR3 beta sequence comprising the sequence CSASEGIGQPQHF (SEQ ID NO: 69) and CDR1 beta and CDR2 beta sequence comprising the CDR1 beta and CDR2 beta sequences respectively of TRBV20-1,
(r). a CDR3 beta sequence comprising the sequence CASSPGLTANVLTF (SEQ ID NO: 70) and CDR1 beta and CDR2 beta sequence comprising the CDR1 beta and CDR2 beta sequences respectively of TRBV27,
(s). a CDR3 beta sequence comprising the sequence CASSQEQLAGPEQYF (SEQ ID NO: 71) and CDR1 beta and CDR2 beta sequence comprising the CDR1 beta and CDR2 beta sequences respectively of TRBV4-2; or
(t). a CDR3 beta sequence comprising the sequence CASSFAGTDTQYF (SEQ ID NO: 126) and CDR1 beta and CDR2 beta sequence comprising the CDR1 beta and CDR2 beta sequences respectively of TRBV27; or, in any of (a) to (t), a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

According to the fifth aspect the isolated anti-cancer T-cell receptor (TCR) or anti-cancer binding fragment thereof may comprise a beta chain variable region comprising any one of:
(a). a CDR3 beta sequence comprising the sequence CASSWAGPVEQYF (SEQ ID NO: 53) and CDR1 beta and CDR2 beta sequences comprising the sequences SGHDY (SEQ ID NO:94) and FNNNVP (SEQ ID NO:111) respectively,
(b). a CDR3 beta sequence comprising the sequence CASSLGLAGNEQYF (SEQ ID NO: 54) and CDR1 beta and CDR2 beta sequences comprising the sequences MNHEY (SEQ ID NO:84) and SMNVEV (SEQ ID NO:98) respectively,
(c). a CDR3 beta sequence comprising the sequence CASSSGLTDTQYF (SEQ ID NO: 55), and CDR1 beta and CDR2 beta sequences comprising the sequences SGHTA (SEQ ID NO:92) and FQGTGA (SEQ ID NO:112) respectively,
(d). a CDR3 beta sequence comprising the sequence CASSLGIISGQPQHF (SEQ ID NO: 56) and CDR1 beta and CDR2 beta sequences comprising the sequences PRHDT (SEQ ID NO:95) and FYEKMQ (SEQ ID NO:113) respectively,
(e). a CDR3 beta sequence comprising the sequence CASSLGGGGYEQYF (SEQ ID NO: 57) and CDR1 beta and CDR2 beta sequences comprising the sequences MNHEY (SEQ ID NO:84) and SMNVEV (SEQ ID NO:98) respectively,
(f). a CDR3 beta sequence comprising the sequence CAISPGEGTDTQYF (SEQ ID NO: 58) and CDR1 beta and CDR2 beta sequences comprising the sequences ENHRY (SEQ ID NO:110) and SYGVKD (SEQ ID NO:114) respectively,
(g). a CDR3 beta sequence comprising the sequence CASSYTGTLSYEQYF (SEQ ID NO: 59) and CDR1 beta and CDR2 beta sequences comprising the sequences MDHEN (SEQ ID NO:85) and SYDVKM (SEQ ID NO:99) respectively,
(h). a CDR3 beta sequence comprising the sequence CASSYVGLGSPLHF (SEQ ID NO: 60) and CDR1 beta and CDR2 beta sequences comprising the sequences MNHEY (SEQ ID NO:84) and SVGAGI (SEQ ID NO:97) respectively,
(i). a CDR3 beta sequence comprising the sequence CATSRDMDPEQYF (SEQ ID NO: 61) and CDR1 beta and CDR2 beta sequences comprising the sequences LNHNV (SEQ ID NO:96) and YYDKDF (SEQ ID NO:115) respectively,
(j). a CDR3 beta sequence comprising the sequence CASSLGGADTQYF (SEQ ID NO: 62) and CDR1 beta and CDR2 beta sequences comprising the sequences MNHEY (SEQ ID NO:84) and SMNVEV (SEQ ID NO:98) respectively,
(k). a CDR3 beta sequence comprising the sequence CASSFLQGTGELFF (SEQ ID NO: 63) and CDR1 beta and CDR2 beta sequences comprising the sequences MNHEY (SEQ ID NO:84) and SMNVEV (SEQ ID NO:98) respectively,
(l). a CDR3 beta sequence comprising the sequence CASRVGILSGELFF (SEQ ID NO: 64) and CDR1 beta and CDR2 beta sequences comprising the sequences MNHEY (SEQ ID NO:84) and SMNVEV (SEQ ID NO:98) respectively,
(m). a CDR3 beta sequence comprising the sequence CATSRDRGWEQFF (SEQ ID NO: 65) and CDR1 beta and CDR2 beta sequences comprising the sequences LNHNV (SEQ ID NO:96) and YYDKDF (SEQ ID NO:115) respectively,
(n). a CDR3 beta sequence comprising the sequence CASSLSGAEAFF (SEQ ID NO: 66) and CDR1 beta and CDR2 beta sequences comprising the sequences MNHEY (SEQ ID NO:84) and SMNVEV (SEQ ID NO:98) respectively,
(o). a CDR3 beta sequence comprising the sequence CASSPSWAHETQYF (SEQ ID NO: 67) and CDR1 beta and CDR2 beta sequences comprising the sequences MNHEY (SEQ ID NO:84) and SMNVEV (SEQ ID NO:98) respectively,
(p). a CDR3 beta sequence comprising the sequence CASSPSGTVYEKLFF (SEQ ID NO: 68) and CDR1 beta and CDR2 beta sequences comprising the sequences MDHEN (SEQ ID NO:85) and SYDVKM (SEQ ID NO:99) respectively,
(q). a CDR3 beta sequence comprising the sequence CSASEGIGQPQHF (SEQ ID NO: 69) and CDR1 beta and CDR2 beta sequences comprising the sequences DFQATT (SEQ ID NO:89) and SNEGSKA (SEQ ID NO:103) respectively,
(r). a CDR3 beta sequence comprising the sequence CASSPGLTANVLTF (SEQ ID NO: 70) and CDR1 beta and CDR2 beta sequences comprising the sequences MNHEY (SEQ ID NO:84) and SMNVEV (SEQ ID NO:98) respectively,
(s). a CDR3 beta sequence comprising the sequence CASSQEQLAGPEQYF (SEQ ID NO: 71) and CDR1 beta and CDR2 beta sequences comprising the sequences LGHNA (SEQ ID NO:90) and YNFKEQ (SEQ ID NO:104) respectively; or
(s). a CDR3 beta sequence comprising the sequence CASSFAGTDTQYF (SEQ ID NO: 126) and CDR1 beta and CDR2 beta sequences comprising the sequences MNHEY (SEQ ID NO:84) and SMNVEV (SEQ ID NO:98), respectively; or, in any of (a) to (t), a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

According to the fifth aspect the isolated anti-cancer T-cell receptor (TCR) or anti-cancer binding fragment thereof preferably comprises: (a) an alpha chain variable region comprising a CDR1 alpha comprising the sequence DRGSQS (SEQ ID NO: 82), a CDR2 alpha comprising the sequence IYSNGD (SEQ ID NO:83), and a CDR3 alpha comprising the sequence CAVSIGFGNVLHC (SEQ ID NO: 15) and a beta chain variable region comprising a CDR1 beta comprising the sequence MNHEY (SEQ ID NO:84), a CDR2 beta comprising the sequence SMNVEV (SEQ ID NO: 98), a CDR3 beta comprising the sequence CASSFAGTDTQYF (SEQ ID NO: 126).

According to a sixth aspect of the present invention there is provided an isolated anti-cancer T-cell receptor (TCR) or anti-cancer binding fragment thereof which comprises:
(i) an alpha chain variable region comprising a complementarity-determining region (CDR3 alpha) comprising or consisting of any one of:

(SEQ ID NO: 19)
CAMSVGFGNVLHC,
(SEQ ID NO: 20)
CAALYNTDKLIF,
(SEQ ID NO: 21)
CAARGAQKLVF,
(SEQ ID NO: 22)
CAATLYNNDMRF,
(SEQ ID NO: 23)
CVVSASNGNKLVF,
(SEQ ID NO: 24)
CAYLVGSNYQLIW,
or
(SEQ ID NO: 25)
CAFMKPNTGNQFYF,

or a variant CDR that has 1, 2, 3, 4 or 5 amino acid variations selected from additions, substitutions and deletions with respect thereto; and/or
(ii) a beta chain variable region comprising a complementarity-determining region (CDR3 beta) comprising or consisting of any one of:

(SEQ ID NO: 53)
CASSWAGPVEQYF,
(SEQ ID NO: 54)
CASSLGLAGNEQYF,
(SEQ ID NO: 55)
CASSSGLTDTQYF,
(SEQ ID NO: 56)
CASSLGIISGQPQHF,
(SEQ ID NO: 57)
CASSLGGGGYEQYF,
(SEQ ID NO: 58)
CAISPGEGTDTQYF,
(SEQ ID NO: 59)
CASSYTGTLSYEQYF
(SEQ ID NO: 60)
CASSYVGLGSPLHF,
(SEQ ID NO: 61)
CATSRDMDPEQYF,
(SEQ ID NO: 62)
CASSLGGADTQYF,
(SEQ ID NO: 63)
CASSFLQGTGELFF,
(SEQ ID NO: 64)
CASRVGILSGELFF,
(SEQ ID NO: 65)
CATSRDRGWEQFF,
(SEQ ID NO: 66)
CASSLSGAEAFF,
(SEQ ID NO: 67)
CASSPSWAHETQYF,
(SEQ ID NO: 68)
CASSPSGTVYEKLFF,
(SEQ ID NO: 69)
CSASEGIGQPQHF,
(SEQ ID NO: 70)
CASSPGLTANVLTF,
(SEQ ID NO: 71)
CASSQEQLAGPEQYF,
or
(SEQ ID NO: 126)
CASSFAGTDTQYF,

or a variant CDR that has 1, 2, 3, 4 or 5 amino acid variations selected from additions, substitutions, and deletions with respect thereto, wherein the TCR or binding fragment binds to the cancer polypeptide antigens Melan-A polypeptide (SEQ ID NO: 72) and IMP2 polypeptide (SEQ ID NO: 74) or antigenic peptide fragments thereof, for example antigenic peptide fragments comprising or consisting of EAAGIGILTV (SEQ ID NO: 75, MelanA), and NLSALGIFST (SEQ ID NO: 77, IMP2).

According to the sixth aspect of the present invention there is provided an isolated anti-cancer T-cell receptor (TCR) or anti-cancer binding fragment thereof which further comprises
(i) an alpha chain variable region further comprising any one of:
(a). a CDR3 alpha sequence comprising the sequence CAMSVGFGNVLHC (SEQ ID NO: 19) and CDR1 alpha and CDR2 alpha sequence comprising the CDR1 beta and CDR2 beta sequences respectively of TRAV12-3,
(b). a CDR3 alpha sequence comprising the sequence CAALYNTDKLIF (SEQ ID NO: 20) and CDR1 alpha and CDR2 alpha sequence comprising the CDR1 beta and CDR2 beta sequences respectively of TRAV29DV5,
(c). a CDR3 alpha sequence comprising the sequence CAARGAQKLVF (SEQ ID NO: 21) and CDR1 alpha and CDR2 alpha sequence comprising the CDR1 beta and CDR2 beta sequences respectively of TRAV23DV6,
(d). a CDR3 alpha sequence comprising the CAATLYNNDMRF (SEQ ID NO:22) and CDR1 alpha and CDR2 alpha sequence comprising the CDR1 beta and CDR2 beta sequences respectively of TRAV13-1,
(e). a CDR3 alpha sequence comprising the sequence CVVSASNGNKLVF (SEQ ID NO: 23) and CDR1 alpha and CDR2 alpha sequence comprising the CDR1 beta and CDR2 beta sequences respectively of TRAV10,
(f). a CDR3 alpha sequence comprising the sequence CAYLVGSNYQLIW (SEQ ID NO: 24) and CDR1 alpha and CDR2 alpha sequence comprising the CDR1 beta and CDR2 beta sequences respectively of TRAV38-2DV8,
(g). a CDR3 alpha sequence comprising the sequence CAFMKPNTGNQFYF (SEQ ID NO: 25) and CDR1 alpha and CDR2 alpha sequence comprising the CDR1 beta and CDR2 beta sequences respectively of TRAV38-1, or, in any of (a) to (g), a variant CDR3 alpha that has 1, 2, 3, 4 or 5 amino acid variations selected from additions, substitutions and deletions with respect thereto, and/or a variant CDR1 alpha and/or CDR2 alpha sequence that has 1, 2, or 3 amino acid variations selected from additions, substitutions and deletions with respect thereto; and/or
(ii) a beta chain variable region comprising any one of:
(a). a CDR3 beta sequence comprising the sequence CASSWAGPVEQYF (SEQ ID NO: 53) and CDR1 beta and CDR2 beta sequence comprising the CDR1 beta and CDR2 beta sequences respectively of TRBV12-4,
(b). a CDR3 beta sequence comprising the sequence CASSLGLAGNEQYF (SEQ ID NO: 54) and CDR1 beta and CDR2 beta sequence comprising the CDR1 beta and CDR2 beta sequences respectively of TRBV27,
(c). a CDR3 beta sequence comprising the sequence CASSSGLTDTQYF (SEQ ID NO: 55) TRBV7-3 and CDR1 beta and CDR2 beta sequence comprising the CDR1 beta and CDR2 beta sequences respectively of TRBV7-3,
(d). a CDR3 beta sequence comprising the sequence CASSLGIISGQPQHF (SEQ ID NO: 56) and CDR1 beta and CDR2 beta sequence comprising the CDR1 beta and CDR2 beta sequences respectively of TRBV13,
(e). a CDR3 beta sequence comprising the sequence CASSLGGGGYEQYF (SEQ ID NO: 57) and CDR1 beta and CDR2 beta sequence comprising the CDR1 beta and CDR2 beta sequences respectively of TRBV27,
(f). a CDR3 beta sequence comprising the sequence CAISPGEGTDTQYF (SEQ ID NO: 58) and CDR1 beta and CDR2 beta sequence comprising the CDR1 beta and CDR2 beta sequences respectively of TRBV10-3,
(g). a CDR3 beta sequence comprising the sequence CASSYTGTLSYEQYF (SEQ ID NO: 59) and CDR1 beta and CDR2 beta sequence comprising the CDR1 beta and CDR2 beta sequences respectively of TRBV28,
(h). a CDR3 beta sequence comprising the sequence CASSYVGLGSPLHF (SEQ ID NO: 60) and CDR1 beta and CDR2 beta sequence comprising the CDR1 beta and CDR2 beta sequences respectively of TRBV6-5,
(i). a CDR3 beta sequence comprising the sequence CATSRDMDPEQYF (SEQ ID NO: 61) and CDR1 beta and CDR2 beta sequence comprising the CDR1 beta and CDR2 beta sequences respectively of TRBV15,
(j). a CDR3 beta sequence comprising the sequence CASSLGGADTQYF (SEQ ID NO: 62) and CDR1 beta and CDR2 beta sequence comprising the CDR1 beta and CDR2 beta sequences respectively of TRBV27,
(k). a CDR3 beta sequence comprising the sequence CASSFLQGTGELFF (SEQ ID NO: 63) and CDR1 beta and CDR2 beta sequence comprising the CDR1 beta and CDR2 beta sequences respectively of TRBV27,
(l). a CDR3 beta sequence comprising the sequence CASRVGILSGELFF (SEQ ID NO: 64) and CDR1 beta and CDR2 beta sequence comprising the CDR1 beta and CDR2 beta sequences respectively of TRBV27,
(m). a CDR3 beta sequence comprising the sequence CATSRDRGWEQFF (SEQ ID NO: 65) and CDR1 beta and CDR2 beta sequence comprising the CDR1 beta and CDR2 beta sequences respectively of TRBV15,
(n). a CDR3 beta sequence comprising the sequence CASSLSGAEAFF (SEQ ID NO: 66) and CDR1 beta and CDR2 beta sequence comprising the CDR1 beta and CDR2 beta sequences respectively of TRBV27,
(o). a CDR3 beta sequence comprising the sequence CASSPSWAHETQYF (SEQ ID NO: 67) and CDR1 beta and CDR2 beta sequence comprising the CDR1 beta and CDR2 beta sequences respectively of TRBV27,
(p). a CDR3 beta sequence comprising the sequence CASSPSGTVYEKLFF (SEQ ID NO: 68) and CDR1 beta and CDR2 beta sequence comprising the CDR1 beta and CDR2 beta sequences respectively of TRBV28,
(q). a CDR3 beta sequence comprising the sequence CSASEGIGQPQHF (SEQ ID NO: 69) and CDR1 beta and CDR2 beta sequence comprising the CDR1 beta and CDR2 beta sequences respectively of TRBV20-1,
(r). a CDR3 beta sequence comprising the sequence CASSPGLTANVLTF (SEQ ID NO: 70) and CDR1 beta and CDR2 beta sequence comprising the CDR1 beta and CDR2 beta sequences respectively of TRBV27,
(s). a CDR3 beta sequence comprising the sequence CASSQEQLAGPEQYF (SEQ ID NO: 71) and CDR1 beta and CDR2 beta sequence comprising the CDR1 beta and CDR2 beta sequences respectively of TRBV4-2; or
(t). a CDR3 beta sequence comprising the sequence CASSFAGTDTQYF (SEQ ID NO: 126) and CDR1 beta and CDR2 beta sequence comprising the CDR1 beta and CDR2 beta sequences respectively of TRBV27; or, in any of (a) to (t), a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

According to the second aspect, the isolated anti-cancer T-cell receptor (TCR) or anti-cancer binding fragment thereof may comprise a beta chain variable region comprising any one of:
(a). a CDR3 beta sequence comprising the sequence CASSWAGPVEQYF (SEQ ID NO: 53) and CDR1 beta and CDR2 beta sequences comprising the sequences SGHDY (SEQ ID NO:94) and FNNNVP (SEQ ID NO:111) respectively,
(b). a CDR3 beta sequence comprising the sequence CASSLGLAGNEQYF (SEQ ID NO: 54) and CDR1 beta and CDR2 beta sequences comprising the sequences MNHEY (SEQ ID NO:84) and SMNVEV (SEQ ID NO:98) respectively,
(c). a CDR3 beta sequence comprising the sequence CASSSGLTDTQYF (SEQ ID NO: 55), and CDR1 beta and CDR2 beta sequences comprising the sequences SGHTA (SEQ ID NO:92) and FQGTGA (SEQ ID NO:112) respectively,
(d). a CDR3 beta sequence comprising the sequence CASSLGIISGQPQHF (SEQ ID NO: 56) and CDR1 beta and CDR2 beta sequences comprising the sequences PRHDT (SEQ ID NO:95) and FYEKMQ (SEQ ID NO:113) respectively,
(e). a CDR3 beta sequence comprising the sequence CASSLGGGGYEQYF (SEQ ID NO: 57) and CDR1 beta and CDR2 beta sequences comprising the sequences MNHEY (SEQ ID NO:84) and SMNVEV (SEQ ID NO:98) respectively,
(f). a CDR3 beta sequence comprising the sequence CAISPGEGTDTQYF (SEQ ID NO: 58) and CDR1 beta and CDR2 beta sequences comprising the sequences ENHRY (SEQ ID NO:110) and SYGVKD (SEQ ID NO:114) respectively,
(g). a CDR3 beta sequence comprising the sequence CASSYTGTLSYEQYF (SEQ ID NO: 59) and CDR1 beta and CDR2 beta sequences comprising the sequences MDHEN (SEQ ID NO:85) and SYDVKM (SEQ ID NO:99) respectively,
(h). a CDR3 beta sequence comprising the sequence CASSYVGLGSPLHF (SEQ ID NO: 60) and CDR1 beta and CDR2 beta sequences comprising the sequences MNHEY (SEQ ID NO:84) and SVGAGI (SEQ ID NO:97) respectively,
(i). a CDR3 beta sequence comprising the sequence CATSRDMDPEQYF (SEQ ID NO: 61) and CDR1 beta and CDR2 beta sequences comprising the sequences LNHNV (SEQ ID NO:96) and YYDKDF (SEQ ID NO:115) respectively,
(j). a CDR3 beta sequence comprising the sequence CASSLGGADTQYF (SEQ ID NO: 62) and CDR1 beta and CDR2 beta sequences comprising the sequences MNHEY (SEQ ID NO:84) and SMNVEV (SEQ ID NO:98) respectively,
(k). a CDR3 beta sequence comprising the sequence CASSFLQGTGELFF (SEQ ID NO: 63) and CDR1 beta and CDR2 beta sequences comprising the sequences MNHEY (SEQ ID NO:84) and SMNVEV (SEQ ID NO:98) respectively,
(l). a CDR3 beta sequence comprising the sequence CASRVGILSGELFF (SEQ ID NO: 64) and CDR1 beta and CDR2 beta sequences comprising the sequences MNHEY (SEQ ID NO:84) and SMNVEV (SEQ ID NO:98) respectively,
(m). a CDR3 beta sequence comprising the sequence CATSRDRGWEQFF (SEQ ID NO: 65) and CDR1 beta and CDR2 beta sequences comprising the sequences LNHNV (SEQ ID NO:96) and YYDKDF (SEQ ID NO:115) respectively,
(n). a CDR3 beta sequence comprising the sequence CASSLSGAEAFF (SEQ ID NO: 66) and CDR1 beta and CDR2 beta sequences comprising the sequences MNHEY (SEQ ID NO:84) and SMNVEV (SEQ ID NO:98) respectively,
(o). a CDR3 beta sequence comprising the sequence CASSPSWAHETQYF (SEQ ID NO: 67) and CDR1 beta and CDR2 beta sequences comprising the sequences MNHEY (SEQ ID NO:84) and SMNVEV (SEQ ID NO:98) respectively,
(p). a CDR3 beta sequence comprising the sequence CASSPSGTVYEKLFF (SEQ ID NO: 68) and CDR1 beta and CDR2 beta sequences comprising the sequences MDHEN (SEQ ID NO:85) and SYDVKM (SEQ ID NO:99) respectively,
(q). a CDR3 beta sequence comprising the sequence CSASEGIGQPQHF (SEQ ID NO: 69) and CDR1 beta and CDR2 beta sequences comprising the sequences DFQATT (SEQ ID NO:89) and SNEGSKA (SEQ ID NO:103) respectively,
(r). a CDR3 beta sequence comprising the sequence CASSPGLTANVLTF (SEQ ID NO: 70) and CDR1 beta and CDR2 beta sequences comprising the sequences MNHEY (SEQ ID NO:84) and SMNVEV (SEQ ID NO:98) respectively,
(s). a CDR3 beta sequence comprising the sequence CASSQEQLAGPEQYF (SEQ ID NO: 71) and CDR1 beta and CDR2 beta sequences comprising the sequences LGHNA (SEQ ID NO:90) and YNFKEQ (SEQ ID NO:104) respectively; or
(t). a CDR3 beta sequence comprising the sequence CASSFAGTDTQYF (SEQ ID NO: 126) and CDR1 beta and CDR2 beta sequences comprising the sequences MNHEY (SEQ ID NO:84) and SMNVEV (SEQ ID NO:98), or, in any of (a) to (t), a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

According to the sixth aspect of the present invention there is provided an isolated anti-cancer T-cell receptor (TCR) or anti-cancer binding fragment thereof which preferably comprises either of:
(a) an alpha chain variable region comprising a CDR1 alpha comprising the sequence NSAFQY (SEQ ID NO:131), a CDR2 alpha comprising the sequence TYSSGN (SEQ ID NO:132), and a CDR3 alpha comprising the sequence CAMSVGFGNVLHC (SEQ ID NO:19) and a beta chain variable region comprising a CDR1 beta comprising the sequence MNHEY (SEQ ID NO:84), a CDR2 beta comprising the sequence SMNVEV (SEQ ID NO:98), a CDR3 beta comprising the sequence CASSLGLAGNEQYF (SEQ ID NO:54); or
(b) an alpha chain variable region comprising a CDR1 alpha comprising the sequence NTAFDY (SEQ ID NO:133), a CDR2 alpha comprising the sequence IRPDVSE (SEQ ID NO:134), and a CDR3 alpha comprising the sequence CAARGAQKLVF (SEQ ID NO:21), and a beta chain variable region comprising a CDR1 beta comprising the sequence MNHEY (SEQ ID NO:84), a CDR2 beta comprising the sequence SVGAGI (SEQ ID NO:97), a CDR3 beta comprising the sequence CASSYVGLGSPLHF (SEQ ID NO:60).

According to a seventh aspect of the present invention there is provided an isolated anti-cancer T-cell receptor (TCR) or anti-cancer binding fragment thereof which comprises any of:
(a) an alpha chain variable region comprising a complementarity-determining region (CDR3 alpha) comprising or consisting of: CAVSDNGNKLVF (SEQ ID NO:119) or a variant CDR that has 1, 2, 3, 4 or 5 amino acid variations selected from additions, substitutions and deletions with respect thereto; and/or a beta chain variable region comprising a complementarity-determining region (CDR3 beta) comprising or consisting of CASSWAGPVEQYF (SEQ ID NO:53), or a variant CDR that has 1, 2, 3, 4 or 5 amino acid variations selected from additions, substitutions and deletions with respect thereto,
(b) an alpha chain variable region comprising a CDR3 alpha sequence comprising the sequence CAVSDNGNKLVF (SEQ ID NO:119) and CDR1 alpha and CDR2 alpha sequence comprising the CDR1 beta and CDR2 beta sequences respectively of TRAV8-6, or a variant CDR3 alpha that has 1, 2, 3, 4 or 5 amino acid variations selected from additions, substitutions and deletions with respect thereto, and/or a variant CDR1 alpha and/or CDR2 alpha sequence that has 1, 2, or 3 amino acid variations selected from additions, substitutions and deletions with respect thereto; and/or a beta chain variable region comprising a CDR3 beta sequence comprising the sequence CASSWAGPVEQYF (SEQ ID NO:53), and CDR1 beta and CDR2 beta sequence comprising the CDR1 beta and CDR2 beta sequences respectively of TRAV12-4, or a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto,
(c) an alpha chain variable region comprising a CDR3 alpha sequence comprising the sequence CAVSDNGNKLVF (SEQ ID NO:119), and CDR1 beta and CDR2 alpha sequences comprising the sequences SSVSVY (SEQ ID NO:135) and YLSGSTLV (SEQ ID NO: 136), respectively, or a variant CDR3 alpha that has 1, 2, 3, 4 or 5 amino acid variations selected from additions, substitutions and deletions with respect thereto, and/or a variant CDR1 alpha and/or CDR2 alpha sequence that has 1, 2, or 3 amino acid variations selected from additions, substitutions and deletions with respect thereto; and/or a beta chain variable region comprising a CDR3 beta sequence comprising the sequence CASSWAGPVEQYF (SEQ ID NO:53), and CDR1 beta and CDR2 beta sequences comprising the sequences SGHDY (SEQ ID NO: 94) and FNNNVP (SEQ ID NO:111), respectively, or a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto; or
(d) an alpha chain variable region comprising a CDR3 alpha sequence comprising the sequence CAVSDNGNKLVF (SEQ ID NO:119), and CDR1 beta and CDR2 alpha sequences comprising the sequences SSVSVY (SEQ ID NO:135) and YLSGSTLV (SEQ ID NO:136), respectively, and/or a beta chain variable region comprising a CDR3 beta sequence comprising the sequence CASSWAGPVEQYF (SEQ ID NO:53), and CDR1 beta and CDR2 beta sequences comprising the sequences SGHDY (SEQ ID NO:94) and FNNNVP (SEQ ID NO:111), respectively;
Wherein, in (a) to (d), the TCR or binding fragment binds to the cancer polypeptide antigens Melan-A polypeptide (SEQ ID NO: 72), BST2 polypeptide (SEQ ID NO: 73) and IMP2 polypeptide (SEQ ID NO: 74) or antigenic peptide fragments thereof, for example antigenic peptide fragments comprising or consisting of EAAGIGILTV (SEQ ID NO: 75, MelanA), LLLGIGILVL (SEQ ID NO: 76, BST2) and NLSALGIFST (SEQ ID NO: 77, IMP2).

Preferably the isolated anti-cancer T-cell receptor (TCR) or anti-cancer binding fragment thereof according to the present invention does not comprise an alpha chain variable region comprising a CDR1 alpha comprising the sequence DRGSQS (SEQ ID NO:82), a CDR2 alpha comprising the sequence IYSNGD (SEQ ID NO:83), and a CDR3 alpha comprising the sequence CAVNVAGKSTF (SEQ ID NO:116), and a beta chain variable region comprising a CDR1 beta comprising the sequence (GTSNPN SEQ ID NO:88) a CDR2 beta comprising the sequence SVGIG (SEQ ID NO: 102), a CDR3 beta comprising the sequence CAWSETGLGTGELFF (SEQ ID NO: 121).

TCR polypeptide sequences of the invention can be obtained and manipulated using the techniques disclosed for example in Green and Sambrook 2012 Molecular Cloning: A Laboratory Manual 4th Edition Cold Spring Harbour Laboratory Press.

### Sequence Comparisons

For the purposes of comparing two closely related polypeptide sequences, the “% sequence identity” between a first sequence and a second sequence may be calculated. Polypeptide sequences are said to be the same as or identical to other polypeptide sequences, if they share 100% sequence identity over their entire length. Residues in sequences are numbered from left to right, i.e. from N- to C-terminus for polypeptides. The terms “identical” or percentage “identity”, in the context of two or more polypeptide sequences, refer to two or more sequences or sub-sequences that are the same or have a specified percentage of amino acid residues that are the same (i.e. at least 85%, 86%, 87%, 88%, 89%, 90%, 91%, 92%, 93%, 94%, 95%, 96%, 97%, 98% or 99% sequence identity over a specified region), when compared and aligned for maximum correspondence over a comparison window (segment of sequence designated to be compared). Suitably, the comparison is performed over a window corresponding to the entire length of the reference sequence. Methods of alignment of sequences for comparison are well-known in the art, for example the BLAST algorithm also performs a statistical analysis of the similarity between two sequences (see, e.g., Karlin & Altschul, 1993, Proc. Nat'l. Acad. Sci. USA 90:5873-5787). Such procedures serve to compare a first, reference sequence to a second, comparison sequence, to determine the degree of identity or the number of additions, substitutions and/or deletions which when made to the first sequence would produce the second sequence.

### Sequence Variants

The term “amino acid” refers to any one of the naturally occurring amino acids, as well as amino acid analogues and amino acid mimetics that function in a manner which is similar to the naturally occurring amino acids. In an embodiment, the amino acid sequence of the TCR or cancer-specific binding fragment thereof is artificial. Without limitation, and as discussed further herein, the TCR or cancer-specific binding fragment thereof may comprise at least one mutation to remove a cysteine residue by replacement with another residue and/or to introduce a cysteine residue by replacement of another residue with cysteine.

In an embodiment, at least one amino acid is substituted, added or deleted relative to the wildtype sequence. In an embodiment, the at least one amino acid is (are) located in a framework region, a CDR or a constant region, particularly in a framework region or a constant region, especially in a constant region. Thus, particularly, any and all additions, substitutions and deletions of amino acids are in a framework region or a constant region, particularly in a constant region, alternatively the at least one amino acid is not located in any CDR.

Variations in sequence can be in the form of amino acid additions, substitutions and deletions, especially substitutions. For example, additions can be at the N and/or C termini of sequences and deletions can be at the N and/or C termini of sequences. Substitutions, i.e. substitution of one amino acid for another at a particular position in a sequence, are suitably conservative substitutions. The following eight groups each contain amino acids that are typically conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins 1984).

Suitably, sequence variations do not significantly adversely affect the ability of the TCR or fragment thereof to bind to its target antigen or epitope, for example to the binding motif of the invention presented on the surface of the cancer or tumour, for example its binding affinity of the variant is 75% or more e.g. 80% or more e.g. 85% or more e.g. 90% or more e.g. 95% or more e.g. 98% or more e.g. 99% or more of that of the TCR that in soluble form has a and β chains of said TCR respectively.

Where sequence variants (e.g. of an α or β chain or a fragment thereof e.g. cancer specific binding fragment, variable domain, a soluble fragment or cancer specific binding fragment, or a CDR) are said to have at least 85% identity to a reference sequence, they may have at least 86%, 87%, 88%, 89%, 90%, 91%, 92%, 93%, 94%, 95%, 96%, 97%, 98% or 99% sequence identity to the reference sequence.

The following variants formed of sequence mutations are contemplated in particular:

Particularly useful mutations are mutations made to one or both chains (i.e. one or both of the α and β chains) of a TCR of the invention to reduce the likelihood that said chain or chains will pair with a chain or chains of the TCR naturally produced by a T-cell. In an embodiment, the constant region of one or preferably both chains is modified. For example, mutations may be made in the constant region of the extracellular region to promote pairing of the recombinant chains and reduce pairing with an endogenous TCR. One such example is the introduction into the constant region of the extracellular region of the α and β chains of a cysteine (C) residue such that the TCR forms a disulphide bridge not present in the naturally produced TCR.

Further useful mutations that may be made include mutations intended to increase the stability of a domain and/or promote correct folding, especially the extracellular domain of a soluble TCR. Other possible mutations include the removal of native cysteine residues (particularly in the instance when new cysteine residues are introduced as mentioned above). Thus, existing C residues may be removed in the extracellular region (particularly within the constant region thereof) of either or both of the chains. For example, an existing C residue in the extracellular region of the β chain within the constant region may be replaced with A, S or T, especially A. An existing C residue in the extracellular region of the α chain within the constant region may also (or instead) be replaced with A, S or T, especially A. Sequences for expression in a bacterial host may be provided with an initial M residue.

### Polynucleotides

According to a further aspect of the invention there is provided a polynucleotide encoding the TCR or anti-cancer binding fragment thereof according to the invention.

In an embodiment, such a polynucleotide may be a chimeric polynucleotide comprising a gene encoding the TCR or anti-cancer binding fragment and a heterologous promoter and/or other transcription control element such as a terminating signal operably linked thereto.

Since a complete TCR comprises an α chain and a β chain, two polynucleotides each encoding a chain of the TCR may be provided or a polynucleotide encoding both chains of the TCR may be provided. Yet further, the two chains of the TCR may be linked by a cleavable peptide linker (e.g. a linker that cleaves in an immune cell, particularly a T-cell; including a “self-cleaving” viral 2A sequence) and the polynucleotide may encode both chains of the TCR and the linker such as nucleic acids.

The terms “nucleic acid” and “polynucleotide” are used interchangeably herein and refer to a polymeric macromolecule made from nucleotide monomers particularly deoxyribonucleotide or ribonucleotide monomers. The term encompasses polynucleotides containing known nucleotide analogues or modified backbone residues or linkages, which are naturally occurring and non-naturally occurring, which have similar properties as the reference polynucleotide, and which are intended to be metabolized in a manner similar to the reference nucleotides or are intended to have extended half-life in the system. Examples of such analogues include, without limitation, phosphorothioates, phosphoramidates, methyl phosphonates, chiral-methyl phosphonates, 2-O-methyl ribonucleotides, peptide-nucleic acids (PNAs). Suitably the term “polynucleotide” refers to naturally occurring polymers of deoxyribonucleotide or ribonucleotide monomers. Suitably the polynucleotides of the invention are recombinant. Recombinant means that the polynucleotide is the product of at least one of cloning, restriction or ligation steps, or other procedures that result in a polynucleotide that is distinct from a polynucleotide found in nature (e.g., in the case of cDNA). In an embodiment the polynucleotide of the invention is an artificial polynucleotide sequence (e.g., a cDNA sequence or polynucleotide sequence with non-naturally occurring codon usage). In one embodiment, the polynucleotides of the invention are DNA. Alternatively, the polynucleotides of the invention are RNA.

DNA (deoxyribonucleic acid) and RNA (ribonucleic acid) refer to polynucleotides having a backbone of sugar moieties which are deoxyribosyl and ribosyl moieties respectively. As used herein, a “corresponding RNA” is an RNA having the same sequence as a reference DNA but for the substitution of thymine (T) in the DNA with uracil (U) in the RNA.

In an embodiment the polynucleotide of the invention is a DNA, including single- or double-stranded DNA and straight-chain or circular DNA (i.e. plasmid DNA). Due to the degeneracy of the genetic code, a large number of different, but functionally identical polynucleotides can encode any given polypeptide. Such polynucleotide variations lead to “silent” (sometimes referred to as “degenerate” or “synonymous”) variants, which are one species of conservatively modified variations. Every polynucleotide sequence disclosed herein which encodes a polypeptide also enables every possible silent variation of the polynucleotide. Accordingly, each silent variation of a polynucleotide that encodes a polypeptide is implicit in each described sequence and is provided as an aspect of the invention.

Codons of the polynucleotide sequences of the invention may be altered in order that sequence variants of the TCR are expressed as discussed above. In an embodiment, up to 5 codons are altered e.g. one, two, three, or four, e.g. one or two e.g. one codons are altered such that a different amino acid is encoded where the codon alteration occurs. Codons encoding residues of the CDRs may be altered alternatively they are not altered. In an embodiment, the polynucleotides of the invention are codon optimised for expression in a human host cell, particularly, an immune cell, particularly a T-cell. The polynucleotide may suitably encode an immature TCR which comprises an N-terminal leader sequence which is removed post translationally by cellular peptidases (such as signal peptidase) to produce the mature form.

### Vectors

According to a yet further aspect of the invention there is provided a vector encoding the TCR or anti-cancer binding fragment of the invention. Specifically, there is provided a vector for delivery of a polynucleotide of the invention to cells (particularly immune cells, such as T-cells) comprising a polynucleotide encoding the TCR or anti-cancer binding fragment of the invention.

As noted above, since a complete TCR comprises an α chain and a β chain, two vectors each comprising a polynucleotide encoding a chain of the TCR may be provided or a vector comprising a polynucleotide encoding both chains of the TCR may be provided. Yet further, the two chains of the TCR may be linked by a cleavable peptide linker (e.g. a linker that cleaves in an immune cell, particularly a T-cell permitting the encoded TCR chains to associate) and the vector may comprise a polynucleotide which encodes both chains of the TCR and the linker.

The, or each vector should suitably comprise such elements as are necessary for permitting transcription of a translationally active RNA molecule in the host cell, such as a promoter and/or other transcription control elements such as an internal ribosome entry site (IRES) or a termination signal. A “translationally active RNA molecule” is an RNA molecule capable of being translated into a protein by the host cell's translation apparatus.

Example promoters to drive transcription of the TCR chains include constitutive promoters such as the cytomegalovirus (CMV) promoter and elongation factor 1α (EF1α) promoter.

The vector may be, for example, a viral vector such as a lentiviral vector. Other examples of viral vectors include vectors derived from γ-retrovirus, adenovirus, adeno-associated virus (AAV), alphavirus, herpes virus, arenavirus, measles virus, poxvirus or rhabdovirus. DNA molecules, for example transposons, may also be suitable vectors to transduce immune cells, such as T-cells with TCR genes. A suitable vector of the invention comprises the aforementioned polynucleotide and is, for example, a viral vector as disclosed above such as a lentiviral vector.

### Production of TCRs

TCRs of the invention and the derivatives described herein (bispecifics or fusion proteins etc) can be obtained and manipulated using the techniques disclosed for example in Green and Sambrook 2012 Molecular Cloning: A Laboratory Manual 4th Edition Cold Spring Harbour Laboratory Press. In particular, artificial gene synthesis may be used to produce polynucleotides followed by expression in a suitable organism to produce polypeptides. A gene encoding a polypeptide of the invention can be synthetically produced by, for example, solid-phase DNA synthesis. In the context of this invention one skilled in the art would understand that the polynucleotide sequences encoding the TCRs and fragments thereof described in this invention could be readily used in a variety of protein production systems, including, for example, viral vectors.

For the purposes of production of polypeptides of the invention in a microbiological host (e.g., bacterial or fungal), polynucleotides of the invention will comprise suitable regulatory and control sequences (including promoters, termination signals etc) and sequences to promote polypeptide secretion suitable for protein production in the host. Similarly, polypeptides of the invention could be produced by transducing cultures of eukaryotic cells with polynucleotides of the invention which have been combined with suitable regulatory and control sequences (including promoters, termination signals etc) and sequences to promote polypeptide secretion suitable for protein production in these cells.

Improved isolation of the polypeptides of the invention produced by recombinant means may optionally be facilitated through the addition of a purification tag at one end of the polypeptide. The polypeptides of the invention may be produced ex vivo in immune cells, such as T-cells as discussed below.

### Immune Cells and Immune Cell Clones

The present invention further provides an immune cell, such as a T-cell, particularly an engineered immune cell such as an engineered T-cell, expressing the TCR or anti-cancer binding fragment according to the invention or comprising the polynucleotide or vector of the invention.

The immune cells and immune cell clones which may express the TCR of the invention or into which the TCR may be transduced include cytotoxic cells such as T-cells, NK-cells and NKT-cells, particularly T-cells. The immune cells and immune cell clones may be cells of the lymphoid lineage including T cells, Natural Killer T (NKT) cells, and precursors thereof including embryonic stem cells, and pluripotent stem cells from which lymphoid cells may be differentiated. Preferably the immune cells and immune cell clones are T cells which can for example include, but are not limited to, helper T cells, cytotoxic T cells, memory T cells (including central memory T cells, stem-cell-like memory T cells or stem-like memory T cells), and effector memory T cells such as TEM cells and TEMRA cells, Regulatory T cells or suppressor T cells, Natural killer T cells, Mucosal associated invariant T cells, TILs (tumour infiltrating lymphocytes) and gamma-delta T cells. Preferably, the immune cells and immune cell clones are T cells optionally CD4+ T cells or a CD8+ T cells. Accordingly the immune cells and immune cell clones may be T-cells, optionally CD4+ T cells or CD8+ T cells, or the immune cells and immune cell clones may be a population of T-cells, optionally CD4+ T cells; or CD8+ T cells, or a mixed population of CD4+ T cells and CD8+ T cells. Suitably the T-cell is a CD8+ T-cell.

According to a further aspect of the invention there is provided an immune cell, particularly a T-cell expressing said TCR or anti-cancer binding fragment of the invention, ideally, in either a soluble or membrane compatible form i.e. having a transmembrane region and intracellular region.

According to a yet further aspect of the invention there is provided an immune cell clone, particularly a T-cell clone expressing said TCR or anti-cancer binding fragment of the invention, ideally, in either a soluble or membrane-compatible form i.e. having a transmembrane region and intracellular region.

In an embodiment the immune cell or immune cell clone, particularly the T-cell or T-cell clone is engineered. For example, the invention provides a immune cell or immune cell clone, particularly a T-cell or T-cell clone wherein the cell or cells of the clone are modified by the introduction (e.g. by transduction) of a heterologous polynucleotide encoding the TCR or anti-cancer binding fragment of the invention or by a vector of the invention.

For example the T-cell clone may be selected from any one of MEL5, MEL8, MEL11, MEL25, MEL31, CLL9, MEL54, MEL55, MEL56, MEL57, MEL58 as disclosed herein and particularly with reference to Tables 1 and 3.

In an embodiment, the immune cell, particularly the T-cell comprises a vector comprising a polynucleotide encoding the TCR of the invention and the immune cell, particularly the T-cell expresses the TCR of the invention.

### Ex Vivo Process

According to an aspect of the invention there is provided an ex vivo process comprising (i) obtaining immune cells, particularly T-cells from a patient, (ii) introducing (e.g. by transduction) a heterologous polynucleotide of the invention or a vector of the invention into the immune cells, particularly T-cells to produce modified immune cells, particularly T-cells which express an anti-cancer T-cell receptor (TCR) or an anti-cancer binding fragment of a TCR according to the invention; and (iii) reintroducing said modified immune cells, particularly T-cells into the patient. There is also provided a process comprising reintroducing modified immune cells, particularly T-cells into a patient wherein said modified immune cells, particularly T-cells are obtained from said patient and modified ex vivo (e.g. by transduction) by the introduction of a heterologous polynucleotide of the invention or by a vector of the invention so that they express an anti-cancer T-cell receptor (TCR) or an anti-cancer binding fragment of a TCR according to the invention. The patient in question suitably is a cancer patient particularly a human cancer patient.

The immune cells, particularly T-cells may optionally be expanded before or (more preferably) after step (ii) above. The immune cells, particularly T-cells may be expanded by multiple methods, e.g. by treatment with a cytokine and/or stimulatory antibody for example IL-2 and/or antibodies against CD3 and CD28.

In a further embodiment which is a variant of the aforementioned processes, the transduced immune cells, particularly T-cells which are administered to the patients were not originally from the same patient (i.e. they are allogeneic cells).

The immune cells, particularly T-cells that are introduced into the patient after modification (e.g. by transduction) may be polyclonal or monoclonal. In the latter case, a particular modified (e.g. transduced) clone is selected for expansion before administering the cells to the patient.

### Method of Treatment

There is also provided a method of treatment of cancer comprising administering to a patient in need thereof modified immune cells, particularly T-cells, according to the invention, preferably wherein the immune cells, particularly T-cells are immune cells, particularly T-cells which have been obtained from said patient and modified ex vivo (e.g. by transduction), optionally ex vivo, by the introduction of a heterologous polynucleotide of the invention or by a vector of the invention so that they express an anti-cancer T-cell receptor (TCR) or an anti-cancer binding fragment of a TCR according to the invention.

There is also provided use of the aforementioned modified (e.g. transduced) immune cells, particularly T-cells in the manufacture of a medicament for the treatment of cancer. In the foregoing methods of treatment the cancer treated is preferably those cancer set out in the section on cancer treatment below.

### Fusion Proteins

As discussed above, it may be convenient to produce the TCR as a fusion protein comprising the α and β chains (or fragments thereof comprising at least one CDR, preferably 3 CDRs per chain) which is cleaved to form the respective a and β chains in a cell (e.g. an immune cell, particularly a T-cell). Hence the two chains or fragments thereof are connected by a cleavable linker.

Suitable cleavable linkers include self-cleaving linkers of the 2A family, which can include

P2A
(ATNFSLLKQAGDVEENPGP, SEQ ID NO: 131),
E2A
(QCTNYALLKLAGDVESNPGP, SEQ ID NO: 132),
F2A
(VKQTLNFDLLKLAGDVESNPGP, SEQ ID NO: 133)
and
T2A (EGRGSLLTCGDVEENPGP, SEQ ID NO: 134).

The linker of the 2A family may be preceded by a furin cleavage site and a SGSG (SEQ ID NO: 135) linker sequence

### Bispecifics

According to a yet further aspect of the invention there is provided a pharmaceutical composition or bispecific comprising said TCR or cell or clone or vector.

Alternatively still, said TCR (or anti-cancer binding fragments thereof) may form part of a bispecific wherein said bispecific includes said TCR, for the purpose of binding to its ligand on a cancer cell, and also an immune cell activating component or ligand that binds and so activates an immune cell such as a Killer T-cell.

In an embodiment, there is provided a bispecific construct comprising the TCR or anti-cancer binding fragment of the invention (such as a soluble TCR) and an immune cell activating component or ligand (such as an antibody protein) that binds to and activates an immune cell, for example such as T-cell e.g. a CD8+ T-cell. For example, the immune cell activating component or ligand activates an immune cell via binding to CD3.

For example, one particular bispecific construct comprises the polypeptide sequences of the TCR or anti-cancer binding fragment of the invention, for example a soluble TCR of the invention, and an antibody protein (particularly an agonist antibody protein) that binds to CD3.

Said antibody protein is suitably an antigen-binding domain of an antibody such as a VH (variable heavy domain from a 4 chain antibody) or a VHH (variable heavy domain from a 2 chain (heavy chain only) antibody such as from Camelid) or is a scFv (i.e. a fusion protein comprising the variable regions of the light and heavy chains of an antibody connected by a short (e.g. 10-25 amino acids) linker).

A number of anti-CD3 agonist antibodies are available in the prior art. For example, blinatumomab which is an approved bispecific T-cell engager (BiTE) product consists of a CD19-targeting antibody (heavy chain scFv) connected to a CD3-targeting agonist antibody (light chain scFv). The CD3-targeting agonist antibody (light chain scFv) component of blinatumomab could therefore be used. The other BiTE products solitomab, pasotuxizumab and ertumaxomab also comprise a CD3-targeting agonist antibody component which could be used in a bispecific of the present invention. The TCR-based bispecific tebentafusp also comprises a CD3-targeting agonist antibody component which could be used in a bispecific of the present invention.

A linker may be provided to link a chain of the TCR to the other part of the bispecific construct e.g. a non-immunogenic linker sequence such as GGGGS (SEQ ID NO: 136).

More generally, there is provided a fusion protein comprising the TCR or anti-cancer binding fragment of the invention and a heterologous protein providing the immune cell stimulating and/or activating activity, for example an antibody, which may be a monoclonal antibody, or antibody fragment or binding domain, for example any of Fab, Fab′, diabody, tribody, minibody, scFv, scFv-Fc, F(ab′)2, VhH, V-NAR, dab, dab-Fc, free-LC, half antibody.

There is also provided a polynucleotide encoding said bispecific, bispecific construct or fusion protein. Said polynucleotide is suitably a DNA, including single or double stranded DNA and straight-chain or circular DNA.

### Compositions

The invention provides a pharmaceutical composition comprising the TCR or anti-cancer binding fragment thereof, polynucleotide, vector, immune cell, immune cell clone, T-cell, T-cell clone, bispecific, bispecific construct or fusion protein of the invention and a pharmaceutically acceptable carrier. Suitably the pharmaceutical composition is formulated under sterile conditions and is suitable for parenteral administration. For parenteral administration, the carrier preferably comprises water and may contain buffers for pH control, stabilising agents e.g., surfactants and amino acids and tonicity modifying agents e.g., salts and sugars.

### Cancer Treatment

According to a further aspect of the invention there is provided a method of treating cancer comprising administering said TCR (or anti-cancer binding fragment thereof) or cell or clone or polynucleotide or vector to an individual to be treated.

There is also provided said TCR (or anti-cancer binding fragment thereof) or cell or clone, for example immune cell, immune cell clone, T-cell, T-cell clone or polynucleotide or vector for use in the treatment of cancer.

There is also provided the use of said TCR or cell or clone, for example immune cell, immune cell clone, T-cell, T-cell clone, or vector in the manufacture of a medicament to treat cancer.

Also, there is provided a method of treating cancer in a subject comprising administering a therapeutically effective amount of an immune cell, immune cell clone, T-cell, T-cell clone, bispecific, bispecific construct, fusion protein or pharmaceutical composition of the invention to the subject.

There is also provided an immune cell, immune cell clone, T-cell, T-cell clone, bispecific, bispecific construct, fusion protein or pharmaceutical composition for use in the treatment of cancer.

There is also provided use of an immune cell, immune cell clone, T-cell, T-cell clone, bispecific, bispecific construct, fusion protein or pharmaceutical composition in the manufacture of a medicament to treat cancer.

Said cancer may include solid tumours and blood cancers, for example cancers selected from lung, melanoma (e.g. skin melanoma), bone, breast, blood (e.g. leukemia), prostate, kidney, bladder, cervical, ovarian and colorectal cancers, and in particular selected from lung, melanoma (e.g. skin melanoma), bone and breast cancers.

BST2 and IMP2 are known to be expressed across various cancers including: liver, breast, pancreas, bone, cervical, kidney and hepatocellular (renal cell carcinoma), testicular, colon, prostate, lung, brain, ovarian, blood cancers, squamous cell carcinoma. In particular, BST2 is associated with renal cell carcinoma, testicular cancer, oral cavity squamous cell carcinoma, glioma and hepatocellular carcinoma, mammary tumor growth and metastasis and multiple myeloma. IMP2 is associated with esophageal adenocarcinoma, pancreatic cancer, Acute Myelocytic Leukemia, breast cancer and hepatocellular carcinoma. Preferably the treatment according to the invention, for example using TCRs, TCR fragments, bispecifics, fusion proteins, TCR-T-cells of the invention includes the treatment of the aforementioned cancers.

MELAN A is overexpressed in most melanomas. The MEL8 TCR-T cells of the invention bind and recognise several distinct cancer types including melanoma, breast, pancreatic, AML and prostate and may be used in the treatment thereof.

Equally well, it is also envisaged that all embodiments of the present invention that may be used to treat cancer may potentially be used to prevent cancer. Thus, corresponding methods and uses and substances for use to prevent cancer are provided as an aspect of the invention.

### Combinations

According to a yet further aspect of the invention there is provided a combination therapeutic for the treatment of cancer comprising:
a) said TCR (or anti-cancer binding fragment thereof) or cell or clone, for example immune cell, immune cell clone, T-cell, T-cell clone, or polynucleotide or vector in combination with
b) a further cancer therapeutic agent.
Accordingly, the TCR (or anti-cancer binding fragment thereof) or cell or clone or polynucleotide or vector of the invention may be administered separately, simultaneously or sequentially with an anti-cancer agent.

More generally, there is provided a pharmaceutical composition comprising:
a) the immune cell, immune cell clone, T-cell, T-cell clone, pharmaceutical composition, bispecific, bispecific construct or fusion protein of the invention; and
b) an anti-cancer agent.

Accordingly, the immune cell, immune cell clone, T-cell, T-cell clone, pharmaceutical composition, bispecific, bispecific construct or fusion protein of the invention may be administered separately, simultaneously or sequentially with an anti-cancer agent.

Further anti-cancer agents that may be included in a combination therapy include immune check point inhibitors e.g. selected from PD-1 inhibitors, such as pembrolizumab, (Keytruda) and nivolumab (Opdivo), PD-L1 inhibitors, such as atezolizumab (Tecentriq), avelumab (Bavencio) and durvalumab (Imfinzi) and CTLA-4 inhibitors such as ipilimumab (Yervoy), other immune stimulants such as interferons (e.g. interferon α, β or γ), steroids e.g. prednisolone and alkylating agents such as platinum-based anti-neoplastic agents e.g. cisplatin, carboplatin and oxaliplatin.

In a preferred method of the invention said TCR, cell, clone or vector is administered in combination with an anti-cancer agent such as, but not limited to, a bispecific.

Other features of the present invention will become apparent from the following examples. Unless stated otherwise, any feature disclosed herein may be replaced by an alternative feature serving the same or a similar purpose

### EXAMPLES

1.1. MM909.24 TIL Infusion Product Responds to Widely Shared HLA a*02:01-Restricted Antigens

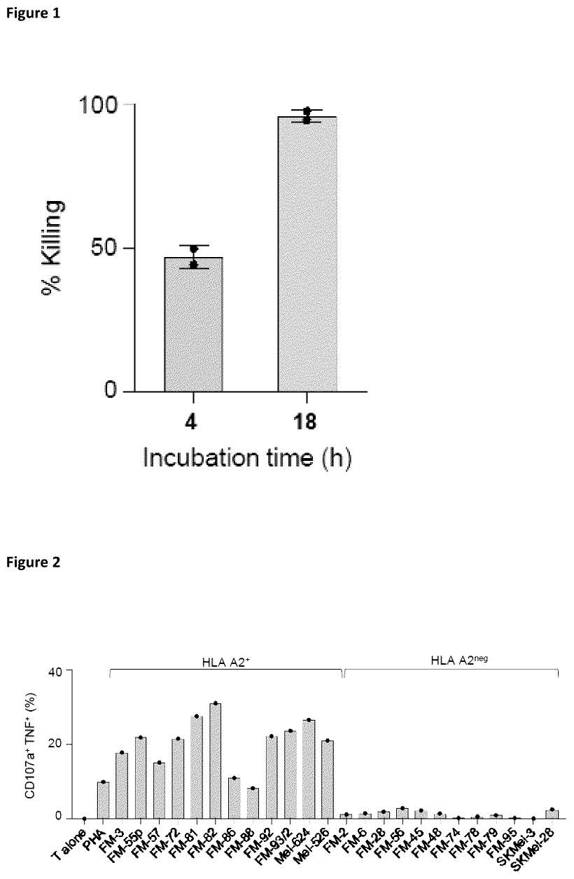

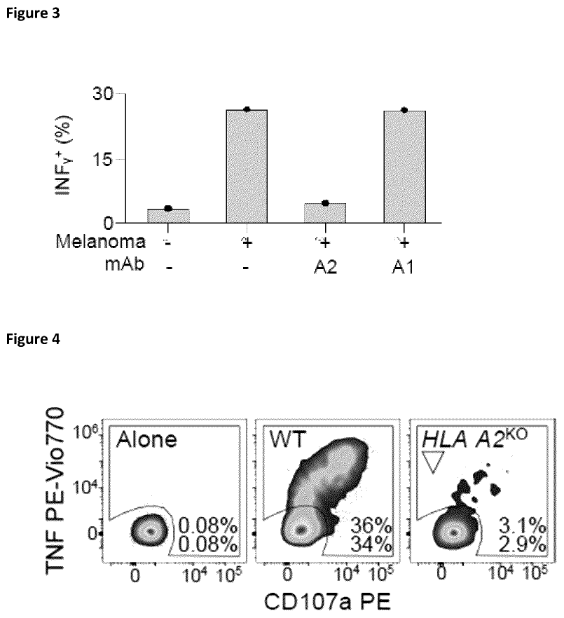

Autologous tumor-specific T-cells were obtained from the TIL infusion product used to induce complete durable remission in HLA A*0201⁺ stage IV malignant melanoma patient MM909.24 at the National Centre for Cancer Immune Therapy, Denmark (CCIT-DK), [Andersen, R. et al. (2016), Clin Cancer Res 22, 3734-3745]. The MM909.24 TIL exhibited robust killing of the autologous melanoma cell line (FIG. 1) and responded well to all HLA A*02:01 melanoma lines tested but did not respond to melanoma lines that lacked HLA A*02:01 expression (FIG. 2), suggesting that the response to the autologous tumor line might be via shared HLA A*02:01-restricted epitopes rather than through patient-specific, mutation-generated neoantigens. Analysis of the MM909.24 TIL response to autologous tumor in the presence of an HLA A*02 blocking antibody and removal of the HLA A*0201 gene from the autologous melanoma line using CRISPR-Cas9 showed that HLA A*02:01-restricted T-cells dominated the response patient tumor (data not shown). FIG. 3 demonstrates that anti-HLA*02 blocking antibody, but not irrelevant anti-HLA*01 antibody, removed almost all the response to the autologous tumor line as measured by intracellular staining of interferon-gamma (INFgamma). FIG. 4 demonstrates that deletion of the HLA A*0201 gene reduced recognition of the autologous melanoma line by over 90%. In conclusion the MM909.24 TIL infusion product responds to widely shared HLA A*02:01-restricted antigens.

### 1.2. MM909.24 TIL and Mel8 Clone Responds to Other Cancer Cell Lines of Differing Origin Through HLA A*0201

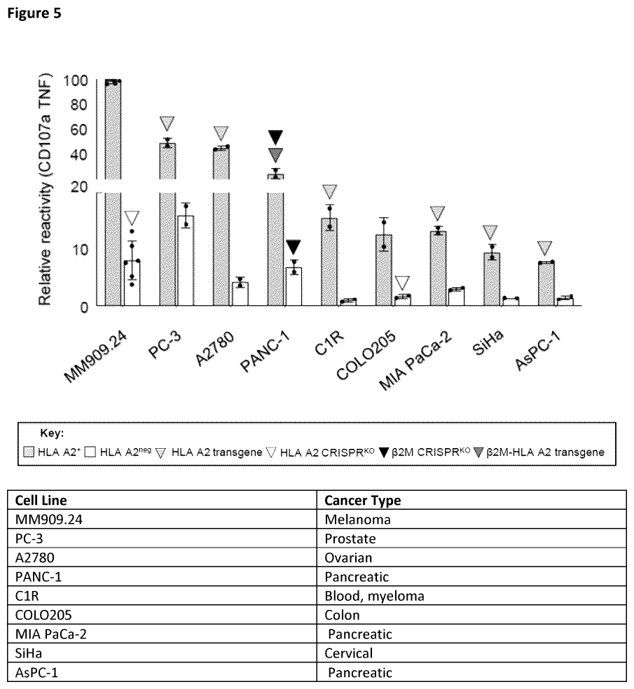

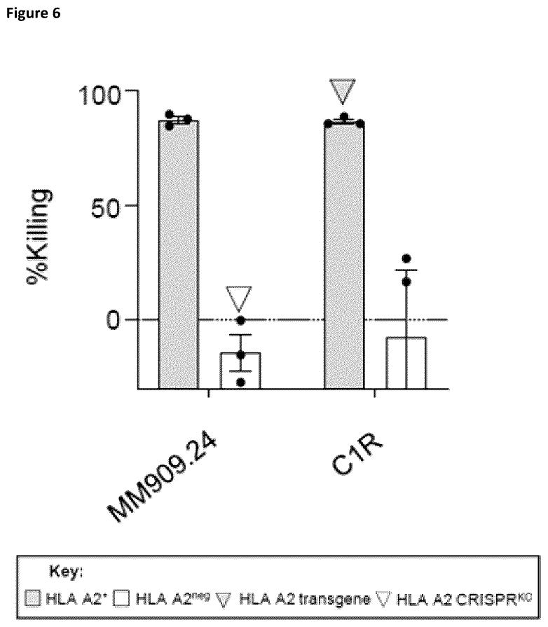

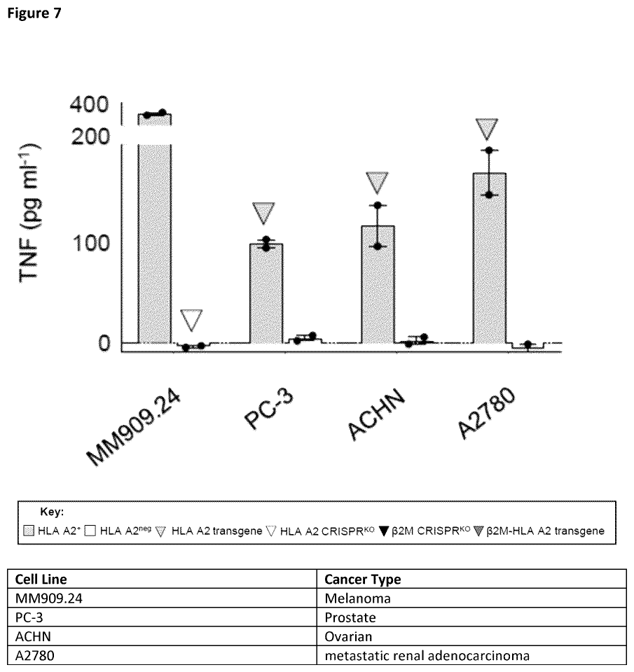

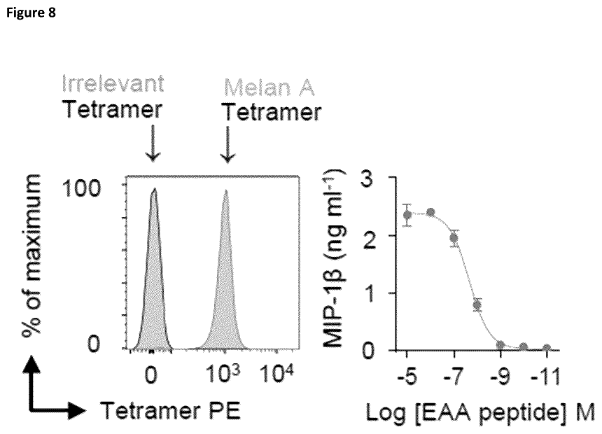

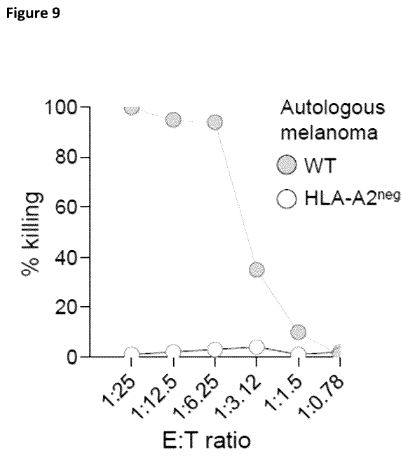

MM909.24 TIL was further investigated to determine whether it responded to other cancer cell lines of differing origin through HLA A*0201, responses were seen to a number of different cancer types other than melanoma from which the TIL was obtained as judged by TIL TFN alpha procution end point (FIG. 5). To pursue the nature of the broad range activity further CD8 T-cells were cloned from TIL MM909.24 infusion product by limiting dilution, one isolated clone, Mel8, demonstrated response to a wide variety of HLA A*0201+ cancer cell lines in addition to melanoma as shown in FIG. 6 and FIG. 7. Peptide-HLA tetramer staining and overnight peptide activation followed by MIP-1beta ELISA using Melan-A26-35 (EAAGIGILTV) peptide [HLAA2-ALWGPDPAAA (preproinsulin15-24) tetramer negative control] confirmed that the MEL8 T-cell clone responded to the Melan A EAAGIGILTV peptide (FIG. 8). MEL8 T-cell clone also exhibits potent killing of the MM909.24 autologous melanoma line via HLA A*02:01 (FIG. 9) assessed by 5 hour incubation in a cytotoxicity chromium release assay against wild type and HLAA2 CRISPR/Cas9 knockout autologous melanoma, no killing is seen for HLAA2 knockout. As Melan A expression is limited to most melanomas and melanosome containing cell types in the skin, mucosa, hair follicles, eye and ear, this suggested that T-cell clone MEL8 might respond to a broadly expressed TAA (tumour associated antigen) through HLA A*02:01 in addition to recognizing the EAAGIGILTV Melan A epitope thereby allowing it to recognize other cancers. Clonotyping confirmed that MEL8 was clonal and expressed single TCR alpha and beta chains with TRAV12-2*02, TRAJ8*01, CDR3alpha CAVQKLVF, and TRBV6-5*01, TRBD2*01, TRBJ2-7*01, CDR3beta CASSYSFTEATYEQYF; this confirmed that the reactivity seen against various cancer types was through a single TCR. CRISPR-Cas9 knockout of Melan A from the MM909.24 melanoma line reduced recognition by MEL8 clone by approximately 10% suggesting recognition of the patient tumor via more than one HLA A*02:01-restricted epitope (data not shown).

### 1.3 Further Epitope Discovery for the MEL8 Clone and Three Further T Cell Clones MEL 11, 25, 31

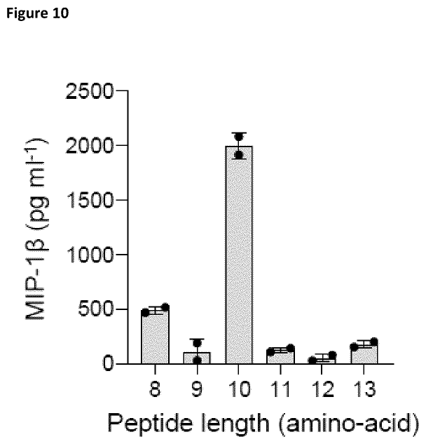

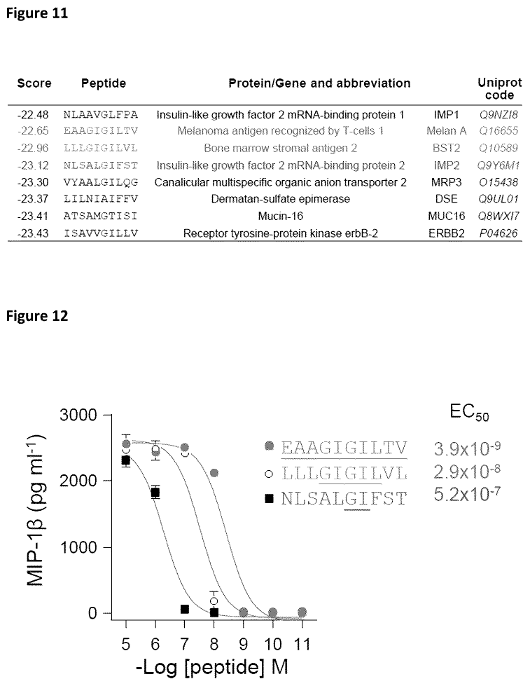

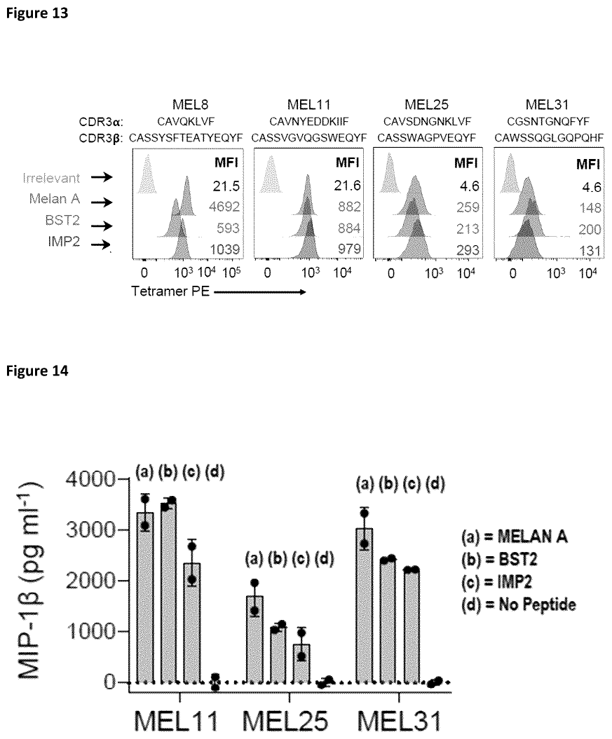

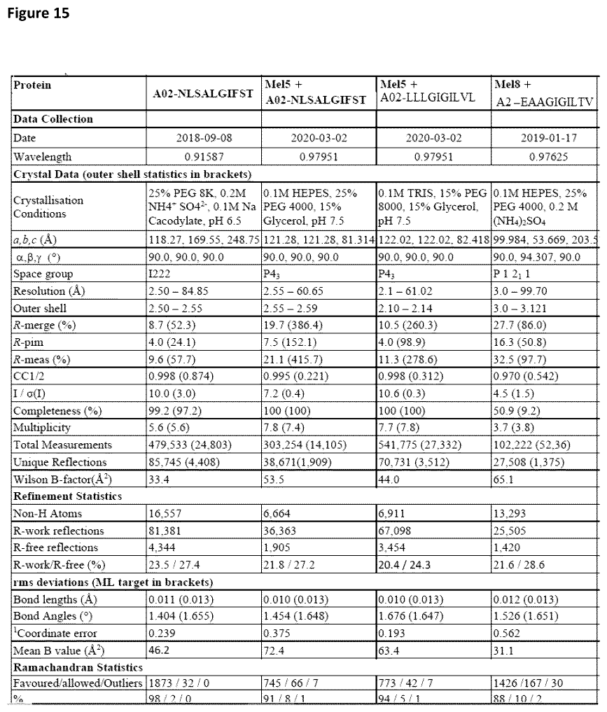

Investigations were carried out to discover further epitopes recognized by the MEL8 T-cell clone which might permit it to recognize cancer types other than melanoma. A sizing scan based on the technique of Ekeruche-Makinde, J. et al. Blood (2013). 121, 1112-1123, showed that MEL8 preferred 10-mer peptides in accordance with it recognizing the EAAGIGILTV peptide (FIG. 10). Screening of potential epitopes was carried out using a CPL (cancer peptide ligand) screening procedure followed by interrogation of results against a cancer-associated proteomics database using a methodology for discovery of peptide epitopes for orphan TCRs that recognize shared, unmutated TAAs. The screening process is based on a T-cell screening methodology using extensive yeast display libraries (e.g. with 9.36×10¹¹ different peptides for a 10-mer library) to successfully predict strong binding sequences in combination with advanced epitope search algorithms [Wooldridge, L. et al. (2012), J Biol Chem 287, 1168-1177; Szomolay, B. et al. (2016), & Immunol Cell Biol; Ekeruche-Makinde, J. et al. (2013), Blood 121, 1112-1123], this permits cancer-specific T-cells to be screened with matched-length CPL. Reactivity of a T-cell clone towards corresponding peptide is determined and the resulting data used to search a human proteome database. The process is further adapted to include graphics processing and proteomic databases to interrogate known CPL data to predict recognized peptides. This approach has proven highly predictive and has several advantages over existing methodologies as the CPL libraries include almost all possible amino acid combinations of a given length (only combinations containing two or more cysteines are excluded); and are unbiased at every position so can be used for any TCR without a priori knowledge of HLA restriction. To improve prediction results, a human cancer proteome database was constructed consisting of full-length protein sequences of TAAs (tumour associated antigen) and described epitopes derived thereof (CANTIGEN; freely available at https://cantigen.cf.ac.uk//launch.php). The core of the CANTIGEN proteomic/peptide database is based around the freely available TANTIGEN database [Olsen, L. R., Cancer Immunol Immunother, 2017 June; 66 (6): 731-735]. Following CPL input to CANTIGEN the returned candidate peptides are scored for likelihood of recognition and to include both known and novel epitopes for testing. CANTIGEN screening of 10-mer CPL data generated using MEL8 produced the ranked list of potential peptide epitopes shown in FIG. 11. MEL8 T-cell clone was demonstrated to recognize three of the top four predicted natural cancer epitopes: EAAGIGILTV from Melan A, LLLGIGILVL from BST2 and NLSALGIFST from IMP2 and stained with HLA A*02:01 tetramers of these peptides, FIG. 12 shows the EC50 of MEL8 peptide activation assessed by MIP-1beta ELISA after overnight co-incubation with the indicated peptides, and FIG. 13 shows tetramer staining of MEL8 (and three other T-cell clones also isolated from the TIL of patient MM909.24). It was noted that the peptides have two common residues at positions 6 (Gly) and 7 (Ile), with the Melan A and BST2 peptides sharing 5 amino acids at positions 5-8 (Gly-Ile-Gly-Ile-Leu). Three further T-cell clonotypes MEL11, MEL25 and MEL31 were also isolated from the TIL used to successfully treat patient MM909.24. All three T-cell clones stained with all three tetramers, Melan A, IMP2 and BST2 and responded to each peptide in activation assays with the respective epitopes (FIG. 13 and FIG. 14). The CDR3 sequences of the four T cell clones are listed in Table 1 below.

### 1.4. MELAN A, BST2, IMP2 Cancer Peptides

BST2 and IMP2 are known to be expressed across various cancers including: liver, breast, pancreas, bone, cervical, kidney and hepatocellular (renal cell carcinoma), testicular, colon, prostate, lung, brain, ovarian, blood cancers, squamous cell carcinoma. The majority of human cancers express BST2, Uhlen, M. et al. (2016) Science 347, 1260419, and high BST2 staining is associated with renal cell carcinoma, testicular cancer, oral cavity squamous cell carcinoma, glioma and hepatocellular carcinoma, mammary tumor growth and metastasis and multiple myeloma. The IMP2 gene is both amplified and overexpressed in many human cancers including esophageal adenocarcinoma, pancreatic cancer, Acute Myelocytic Leukemia, breast cancer and hepatocellular carcinoma. MELAN A is overexpressed in most melanomas.

1.5. Structural Determination of the Shared x-x-x-A/G-I/L-G-I-x-x-x Recognition Motif

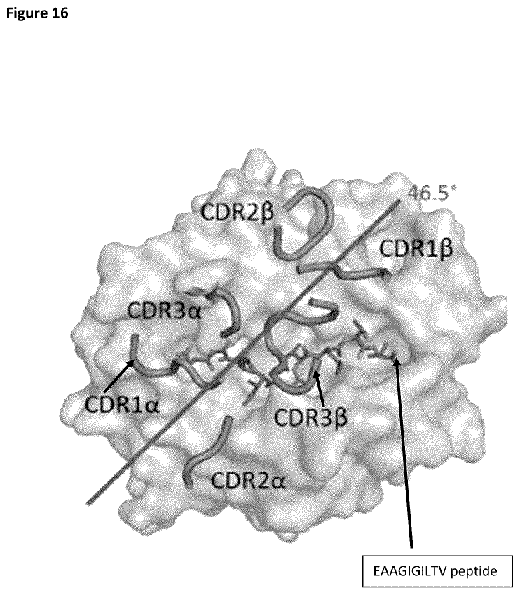

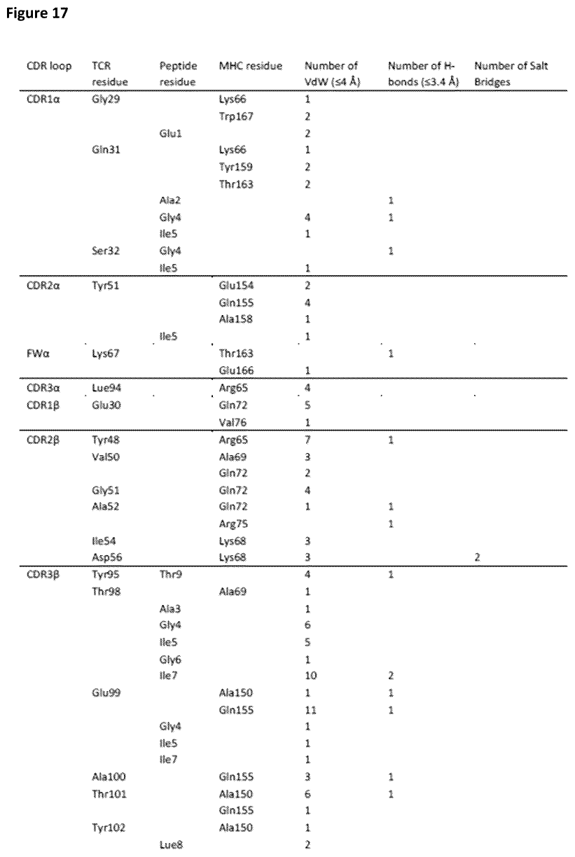

Crystals of the MEL8 TCR in complex with HLA A*02:01-EAAGIGILTV (MELAN A epitope) were produced according to the conditions of FIG. 15, and diffraction data collected from the crystals was obtained and processed according to the statistics shown in FIG. 15. Graphical rendering of the determined structure indicated that the MEL8 TCR CDR1 alpha and CDR3 beta loops are in close proximity to the EAAGIGILTV peptide, FIG. 16 shows a top-down view of EAAGIGILTV peptide (sticks) presented by HLA A*02:01 (grey, shown as surface). MEL8 CDR loops (noodle) are shown as cartoon, a crossing angle is indicated by the grey line. Examination of the bonds between the EAAGIGILTV peptide and the MEL8 TCR show many contacts (54% of Van der Waals and 66% of hydrogen bonds) involve peptide residues Gly4 and Ile7. By contrast, peptide residues Glu1, Ile5 and Leu8 contribute less interactions (24% of Van der Waals and no hydrogen bonds), despite their protrusion towards the MEL8 TCR (FIG. 17).

A comparison between the MEL8-HLA-A*02:01-EAAGIGILTV structure and the previously determined MEL5-HLA-A*02:01-EAAGIGILTV structure [Madura, F. et al. (2015). Eur J Immunol 45, 584-591], shows a conserved CDR1α conformation (both TCRs exhibit TRAV12-2 gene usage but different gene/CDR3 compositions), while the differing CDR3 loop compositions results in differing CDR3 conformations between the MEL8 and MEL5 TCRs, however in both cases the central interactions made with the peptide residues Gly4 and Ile7 remains consistent between both TCRs.

TABLE 1
Multiprong clone chain usage and sequence details

| | | CDR1 | CDR2 | | | CDR1 | CDR2 | | |
|---|---|---|---|---|---|---|---|---|---|
| TCR | TRAV | alpha | alpha | CDR3 alpha | TRBV | beta | beta | CDR3 beta | Target |
| MEL8 | 12- | DRGSQS | IYSNGD | CAVQKLVF | 6- | MNHEY | SVGAGI | CASSYSFTEATYE | MELA/ |
| | 2*01 | SEQ ID | SEQ ID | SEQ ID NO: 1 | 5*01 | SEQ ID | SEQ ID | QYE | BST2/ |
| | | NO: 82 | NO: 83 | | | NO: 84 | NO: 97 | SEQ ID NO 28 | IMP2 |
| MEL5 | 12- | DRGSQS | IYSNGD | CAVNVAGKSTF | 30*02 | GTSNPN | SVGIG | CAWSETGLGTGE | MELA/ |
| | 2*01 | SEQ ID | SEQ ID | SEQ ID NO: | | SEQ ID | SEQ ID | LFF | BST2/ |
| | | NO: 82 | NO: 83 | 116 | | NO: 88 | NO: 102 | SEQ ID NO 121 | IMP2 |
| CLL9 | 12- | DRGSQS | IYSNGD | CAVGAGGFKTIF | 2*02 | SNHLY | FYNNEI | CASSESPLSGNE | MELA/ |
| | 2*01 | SEQ ID | SEQ ID | SEQ ID NO: | | SEQ ID | SEQ ID | QYF | BST2/ |
| | | NO: 82 | NO: 83 | 117 | | NO: 127 | NO: 128 | SEQ ID NO 122 | IMP2 |
| MEL11 | 12- | DRGSQS | IYSNGD | CAVNYEDDKIIF | 9 | SGDLS | HYYNGE | CASSVGVQGSWEQ | MELA/ |
| | 2*01 | SEQ ID | SEQ ID | SEQ ID NO: | | SEQ ID: | SEQ ID | YE | BST2/ |
| | | NO: 82 | NO: 83 | 118 | | NO: 129 | NO: 130 | SEQ ID NO 123 | IMP2 |
| MEL25 | 8-6 | SSVSVY | YLSGSTLV | CAVSDNGNKLVF | 12-4 | SGHDY | ENNNVP | CASSWAGPVEQYE | MELA/ |
| | | SEQ ID | SEQ ID | SEQ ID NO: | | SEQ ID | SEQ ID | SEQ ID NO 53 | BST2/ |
| | | NO: 135 | NO: 136 | 119 | | NO: 94 | NO: 111 | | IMP2 |
| MEL31 | 12- | DRGSQS | IYSNGD | CGSNTGNQFYF | 30 | GTSNPN | SVGIG | CAWSSQGLGQPQHF | MELA/ |
| | 2*01 | SEQ ID | SEQ ID | SEQ ID NO: | | SEQ ID | SEQ ID | SEQ ID NO 34 | BST2/ |
| | | NO: 82 | NO: 83 | 120 | | NO: 88 | NO: 102 | | IMP2 |

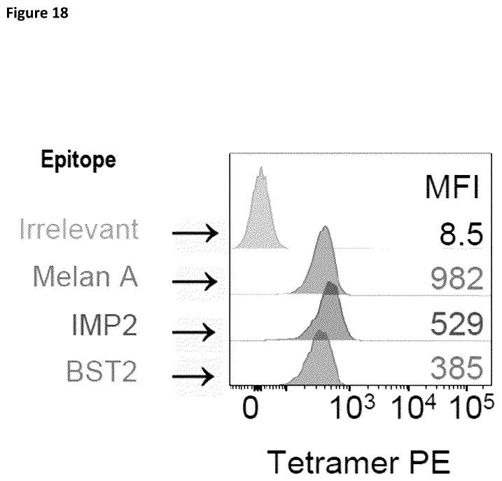

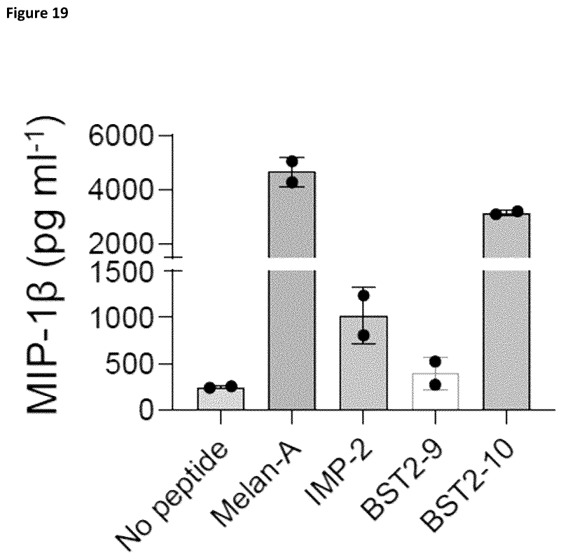

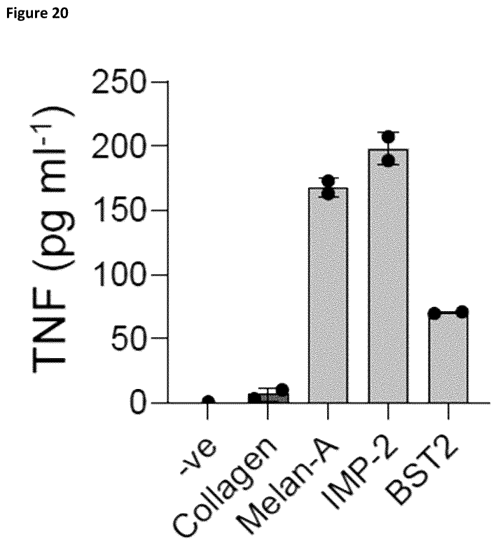

A further investigation was conducted to determine whether the MEL5 T-cell could also recognize the identified BST2 and IMP2 epitopes recognized by MEL8. MEL5 T-cell stained well with tetramers showing peptide recognition of Melan A (EAAGIGILTV), BST2 (LLLGIGILVL) and IMP2 (NLSALGIFST), and showed reaction to the peptides in activation assays (FIGS. 18 & 19), furthermore HLA A*02:01⁺ targets expressing Melan A (EAAGIGILTV), or BST2 (LLLGIGILVL) or IMP2 (NLSALGIFST) were recognized (FIG. 20) confirming that all three peptides were processed and presented at the target cell surface for recognition by Mel5 TCR.

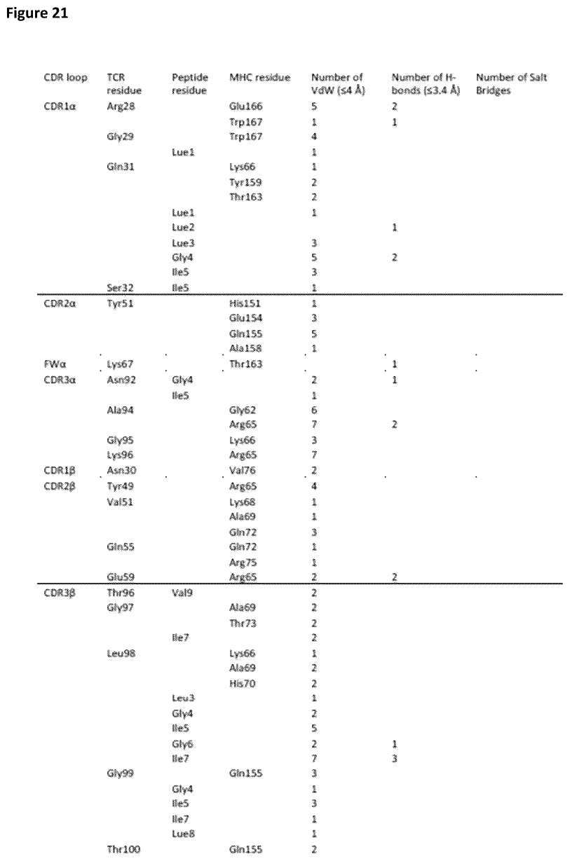

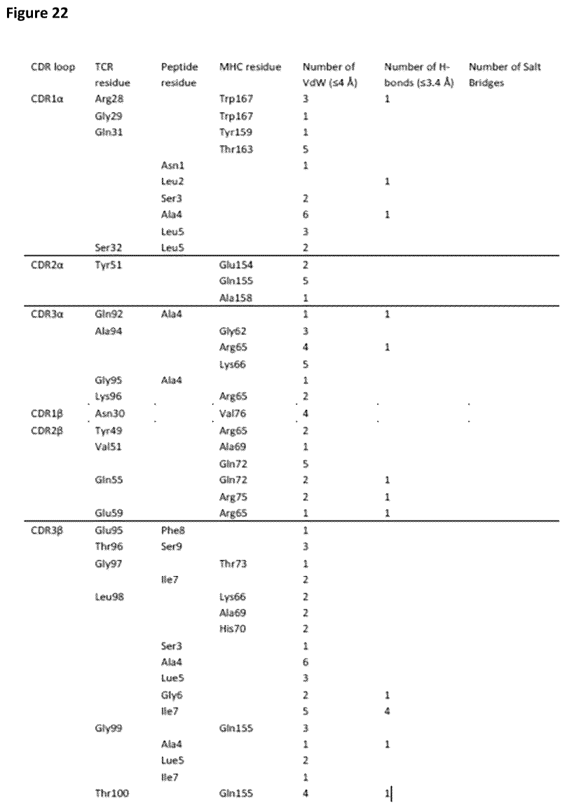

Further crystals were produced of the MEL5 TCR in complex with the BST2 and IMP2 epitopes that diffracted to 2.1 and 2.55 Å respectively according to the conditions recorded in FIG. 15. Overlay of the Melan-A, BST2 and IMP2 epitopes within the complex structures showed that they adopted a similar conformation and an observed conserved TCR docking mechanism despite the different bound peptide sequences. The molecular interaction of MEL5 TCR binding recognition with each of the EAAGIGILTV, LLLGIGILVL and NLSALGIFST epitopes is conserved and is directed on the shared x-x-x-A/G-L/I-x-I-x-x-x motif identified for Mel8-EAAGIGILTV demonstrating that MEL5 TCR cross-reactivity is driven by molecular commonality of structural features between the three epitopes (FIGS. 17, 21, 22) permitting MEL5 T-cells to target cancer via epitopes from three different TAA. Additionally the 3D structure of the unbound HLA A*02:01-NLSALGIFST monomer was determined to 2.5 Å resolution, comparison to the HLA A*02:01-NLSALGIFST MEL5 TCR structure indicated that no conformation shifts occurred within the peptide upon interaction with MEL5, (efforts to obtain crystals of HLA A*02:01-LLLGIGILVL monomers were unsuccessful).

TABLE 2
Multiprong peptide epitope motif and comparison
of key contacts at P4, P5, P7

| | Amino Acid Position | | | | | | | | | |
|---|---|---|---|---|---|---|---|---|---|---|
| Peptide | P1 | P2 | P3 | P4 | P5 | P6 | P7 | P8 | P9 | P10 |
| MelanA | E | A | A | G | I | G | I | L | T | V |
| BST2 | L | L | L | G | I | G | I | L | V | L |
| IMP2 | N | L | S | A | L | G | I | F | S | T |

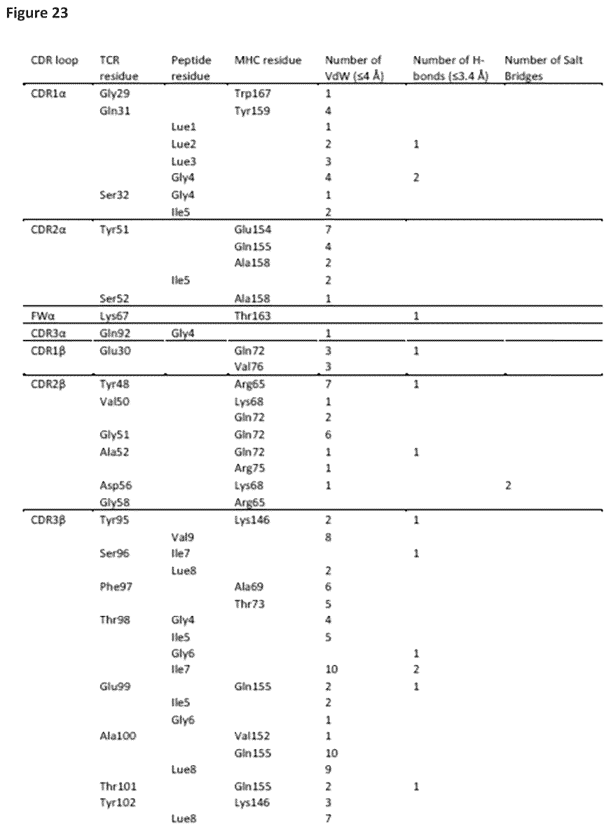

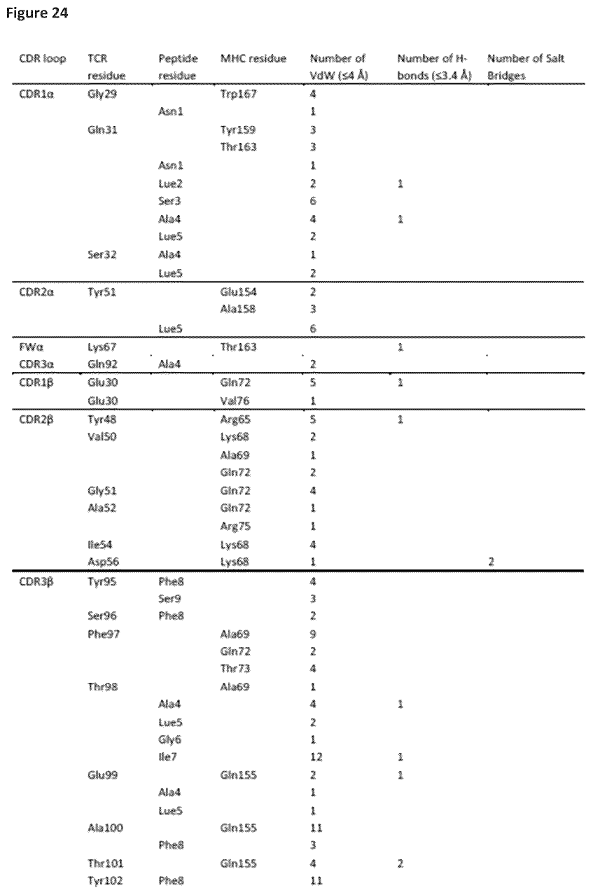

The MEL5 structural ligand data was used to perform molecular dynamics homology modelling of the MEL8 TCR in complex with the BST2 and IMP2 epitopes (MEL8-A2 A*02:01-LLLGIGILVL and the MEL8-A2 A*02:01-NLSALGIFST). Analysis of the modelled interactions between the MEL8 TCR and the cancer epitopes show that interactions involving the P4 and P7 hotspot account for 24% and 39% of the interactions involving the BST2 and IMP2 epitopes respectively (FIGS. 23 & 24) suggesting molecular commonality as a mechanism providing MEL8 cross-reactivity with epitope regions with the shared x-x-x-A/G-L/I-x-I-x-x-x motif (Table 2).

### 1.6. Recognition of Each TAA by Multipronged T-Cells is Additive

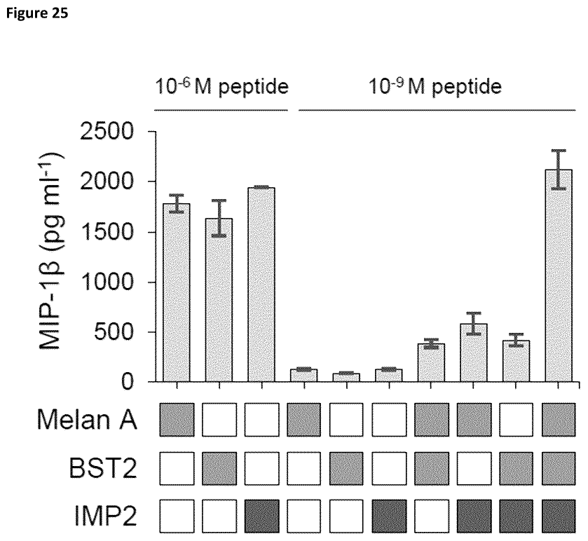

Overnight activation of MEL8 T-cell clone co-incubated with peptides and HLAA2+T2 antigen presenting cells was assessed by MIP-1betaELISA. Melan-A26-35 (EAAGIGILTV), BST2222-31 (LLLGIGILVL) and IMP2367-376 (NLSALGIFST) peptides were used alone or combined. The data showed that recognition of EAAGIGILTV, LLLGIGILVL and NLSALGIFST epitopes is additive. While each peptide was recognized well when targets were pulsed with 10⁻⁶ M peptide, MEL8 T-cells did not recognize targets pulsed with 10⁻⁹ M of each peptide, however peptide pulsing with two peptides at 10⁻⁹ M increased recognition by ˜5-fold. Addition of all three peptides at 10⁻⁹ M, a combined exogenous concentration of just 3×10⁻⁹ M, significantly enhanced target recognition (FIG. 25).

### 1.7. Advantages of Multiprong Over Monospecific TCRs

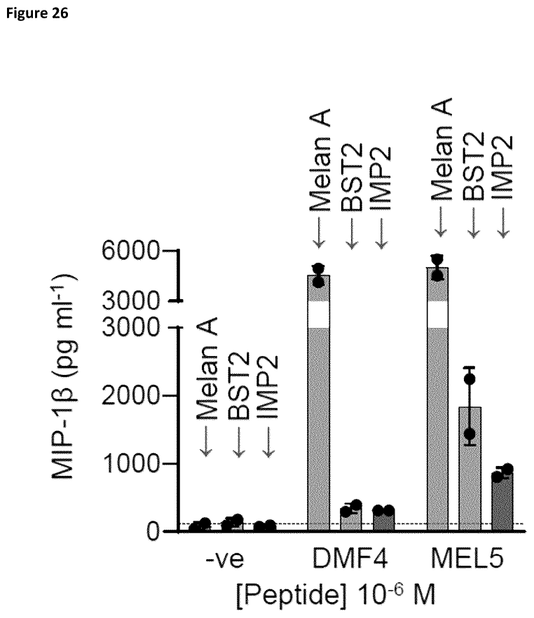

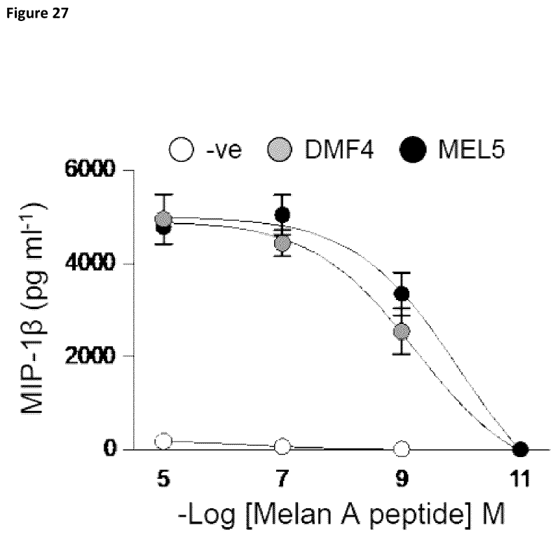

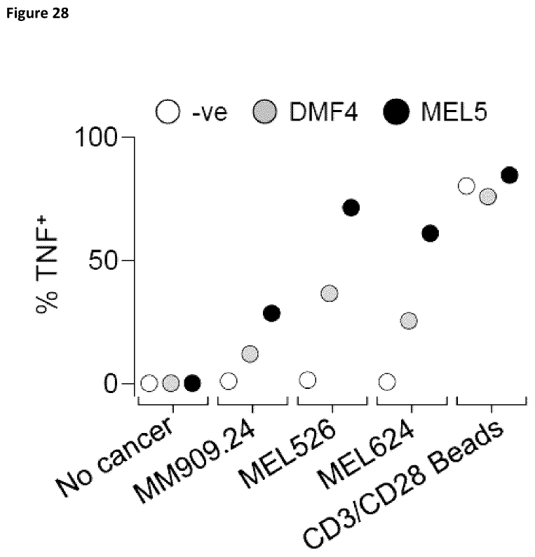

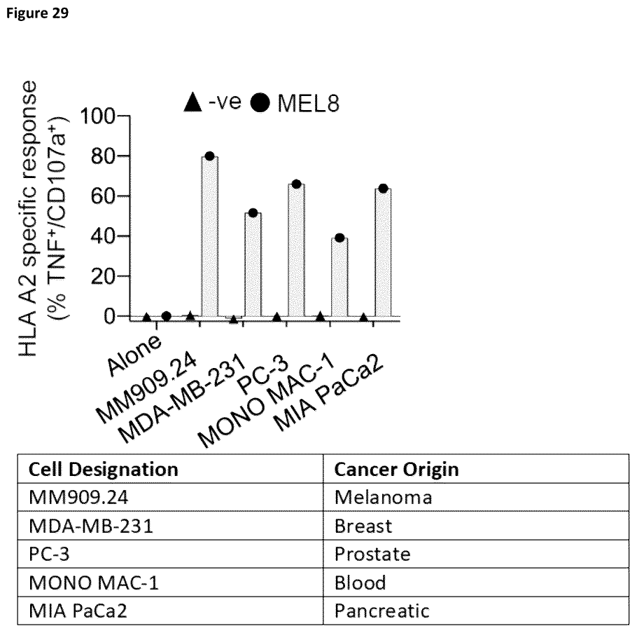

It was determined that CD8 T-cells expressing the clinically relevant Melan A-specific DMF4 TCR [Morgan, R. A. et al. (2006) Science 314, 126-129] only responded to the Melan A peptide (FIG. 26) and not to the identified antigens from BST2 and IMP2. It was further determined that whereas CD8 T-cells transduced with DMF4 or MEL5 TCRs both responded similarly to a titration of with wildtype 10-mer Melan A-derived peptide EAAGIGILTV equally (FIG. 27), CD8 T-cells transduced with the MEL5 TCR responded to the HLA A*02:01 each of the three melanoma lines MM909.24, MEL526 and MEL624 [Marincola, F. M. et al., (1994). J Immunother Emphasis Tumor Immunol 16, 13-23] better than those transduced with the DMF4 TCR (FIG. 28). It was found that melanomas engineered to lack both Melan A and BST2 were recognized by MEL8 but not DMF4. This indicates that the enhanced response of MEL8 correlated with its broader scope of recognition of the melanoma cell cancer antigen population. In conclusion, these data show that a multipronged TCR such as MEL8 recognizing multiple cancer antigens is better equipped at preventing tumor escape mechanisms that involve the loss of protein expression from cancer cells. The data indicate that a multipronged TCR is more sensitive to cancer cells expressing multiple antigens than a regular monospecific TCR. Advantageously, MEL8 TCR-T cells were able to recognize several distinct cancer types; melanoma, breast, pancreatic, AML and prostate (FIG. 29), consequently recognition of multiple tumor-associated antigens (TAA) by a single TCR may help curb cancer immune evasion and provide means for treating multiple cancer types.

### 1.8. Safety of Multipronged T-Cells

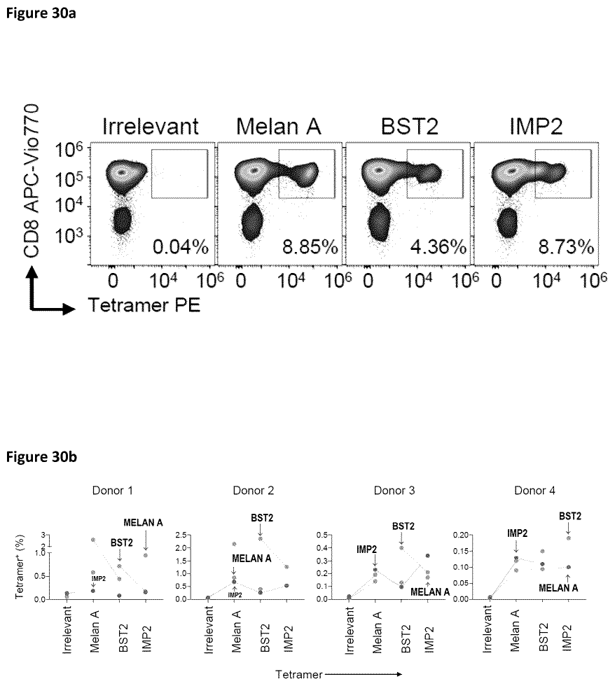

The MEL8 clonotype was isolated from the TIL used to successfully treat patient MM909.24 and was present in blood after TIL infusion without indications of pathology due to BST2 or IMP2 targeting. This indicates that the Mel8 activated T-cell clonotype must be safe in vivo. To further examine safety, we stained the TIL infusion product used to successfully treat patient MM909.24 with HLA A*02:01-EAAGIGILTV, HLA A*02:01-LLLGIGILVL and HLA A*02:01-NLSALGIFST tetramers. 8.85%, 4.36% and 8.73% of the TIL stained with the respective tetramers (FIG. 30a) confirming that patient MM909.24 was infused with an activated T-cell product containing T-cells with each specificity. We conclude that BST2 and IMP2-specific T-cells exhibit a promising safety profile even when transfused into cancer patients in activated form in a large number (>4.7×10⁹ and 9.6×10⁹ activated T-cells with each specificity were transferred to patient MM909.24 based on one dimensional tetramer staining in FIG. 30a).

1.9. Isolation of Further Multiprong T-Cell Clonotypes Through T Cell Priming with Melan a, BST2 and IMP2 Peptides

Purified CD8+ T-cells from PBMC samples of four normal HLAA2 donors were primed with Melan-A26-35 (EAAGIGILTV), BST222-31 (LLLGIGILVL) or IMP2366-376 (NLSALGIFST) peptides for 4 weeks, then stained with MelanA, BST2 and IMP2 tetramers, or with irrelevant tetramer made with ILAKFLHWL (hTERT540-548). It was found that multipronged T-cells that bind Melan A, BST2 and IMP2 tetramers could be primed from these PBMCs FIG. 30b, cells were sorted with gates were set on single lymphocytes and live CD3+CD14neg CD19neg cells. The percentage of tetramer positive cells is shown for each gate. Overall, BST2 or Melan A seemed to be more potent than IMP2 for T-cell priming, it appears that each individual possesses T-cell clonotypes that have potential to simultaneously recognize epitopes from Melan A, BST2 and IMP2 although such clonotypes seem to be in the minority of those with capacity to respond to Melan A, BST2 or IMP2 epitopes.

1.10. Isolation of Further Multiprong Multiprong T-Cells from MM909.24 TIL Product

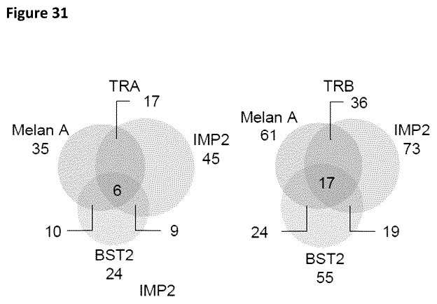

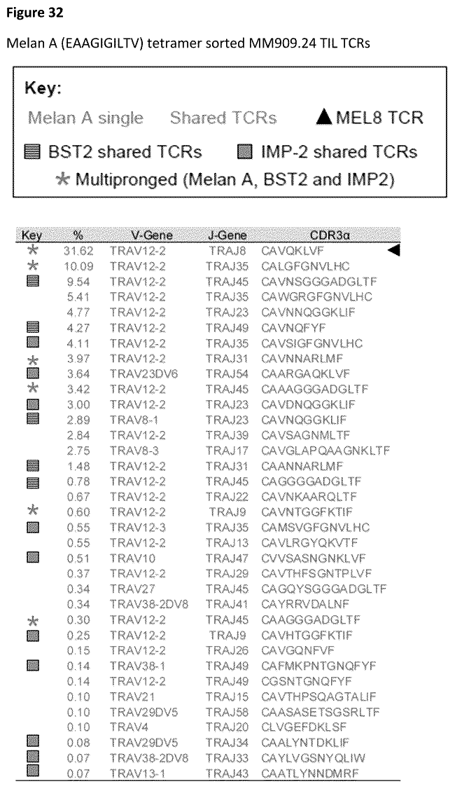

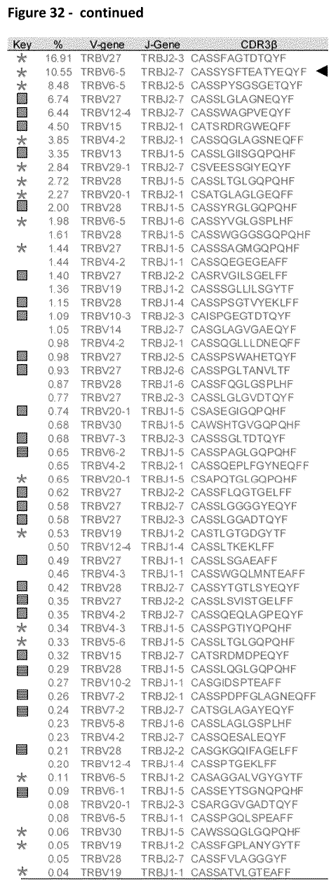

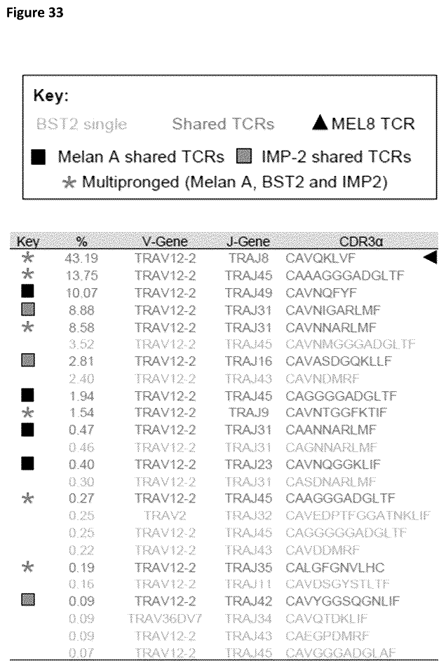

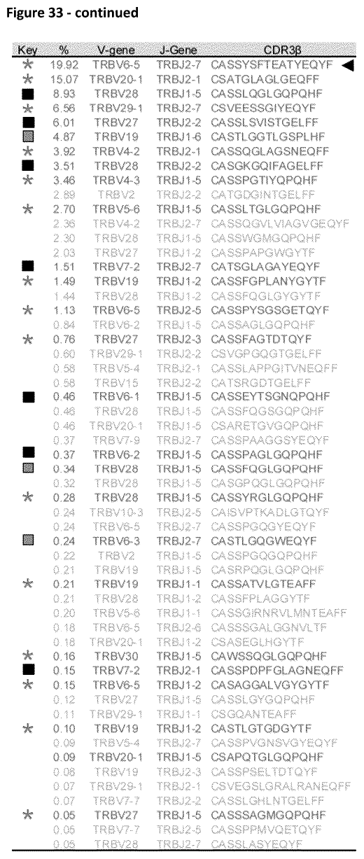

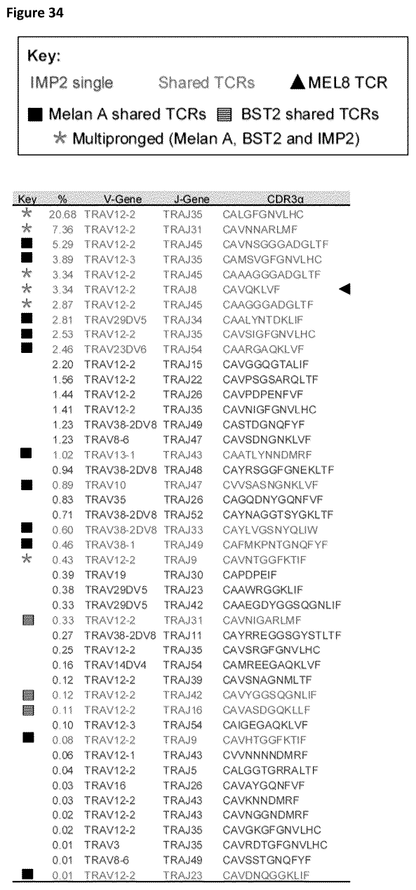

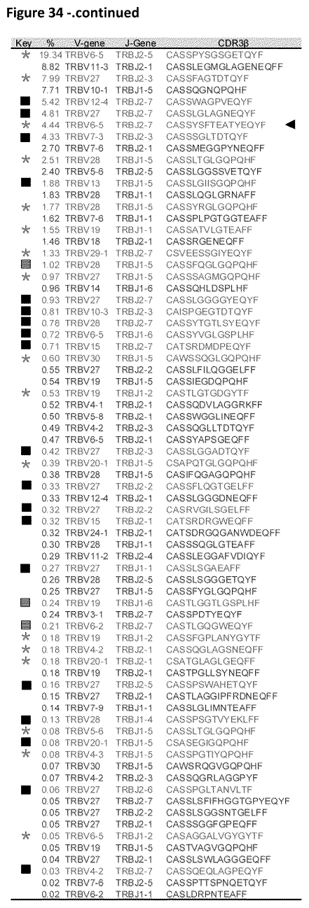

An investigation was carried out to isolate and sequence selected T-cells within the MM909.24 TIL product that stain with HLA A*02:01-EAAGIGILTV, HLA A*02:01-LLLGIGILVL or HLA A*02:01-NLSALGIFST tetramers to characterize families of TCRs within the population that exhibit the ability to recognize more than one of these antigens. FIG. 30a shows the staining of TIL from melanoma patient MM909.24 using HLAA2 MelanA, BST2 and IMP2 tetramers and FIG. 31 shows a Venn diagram illustration of alpha (TRA) and beta (TRB) TCR chains from these Melan A, BST2 and IMP2 tetramer sorts. MEL8 TCR appeared in all three sorts, further T-cells within the MM909.24 TIL product that stained with HLA A*02:01-EAAGIGILTV, HLA A*02:01-LLLGIGILVL or HLA A*02:01-NLSALGIFST tetramers were sequenced to build a picture of which TCRs in each population exhibited the ability to recognize more than one of these antigens (FIG. 31). Overall, the tetramer double populations expressed 75 TCR alpha and 126 TCR beta chains, the V gene, J gene and CDR3 usage is shown in FIGS. 32, 33 & 34. Clonotypes were isolated. The MEL8 clonotype was present in cell populations staining with HLA A*02:01-EAAGIGILTV and HLA A*02:01-LLLGIGILVL tetramers and within the cells that stained with HLA A*02:01-NLSALGIFST. Further T cell clonotypes with 6 TCR alpha and 17 TCR beta were discovered within the MM909.24 TIL infusion product which also stained with all three tetramers. The identified T-cell clonotypes that responded to BST2 and IMP2 epitopes made up the majority of the response within these TIL and had persisted thereby suggesting that such T-cells that respond to BST2 and IMP2 epitopes are safe and have a role in cancer clearance. As both BST2 and/or IMP2 are highly expressed by most cancer types.

1.11. Isolation of Multipronged T-Cells from Non-Melanoma Cancers

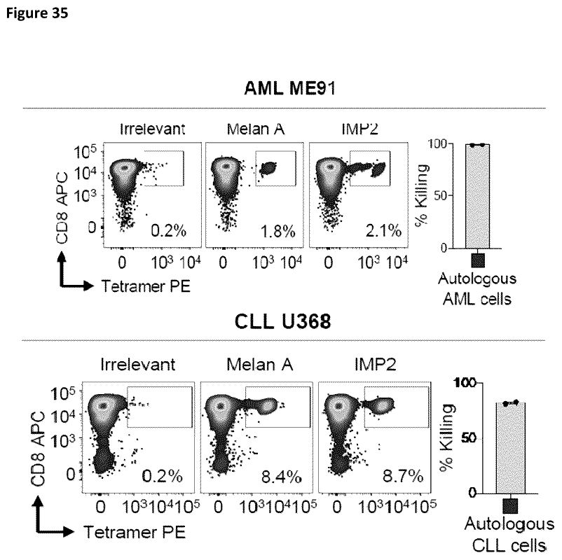

It was further discovered that expanded populations of IMP2-specific T-cells could be obtained from the blood of cancer patients having different cancer types to melanoma. CD8 T-cells were isolated from Acute Myeloid Leukemia (AML) patient ME91 and cultured with cytokines for 4 weeks ex-vivo (2 weeks with IL-2, then IL-2+IL-15) and stained using HLAA2 MelanA and IMP2 tetramers (FIG. 35 [upper]); the bar graph shows data from a flow based killing assay of the IMP2/MelanA T-cell line versus primary autologous AML cancer cells, (gates were set on single lymphocytes and live CD8+CD3+ cells. The data shows that more than 2% of this patient's T-cells recognized the IMP2 epitope in the absence of any specific stimulation through the TCR and exhibited good killing of primary autologous AML cells, 1.8% also stained with Melan A tetramer suggesting T-cell populations that recognized both antigens. The IMP2-specific T-cell line from this donor exhibited good killing of primary autologous AML cells.

In a second experiment IMP2 and Melan A specific T-cells were also isolated from Patient U368 suffering chronic lymphocytic leukemia (CLL). Purified CD8+ T-cells from CLL patient U386 which were stimulated with peptide and cultured with cytokines for 4 weeks (2 weeks with IL-2, then IL-2+IL-15) and stained with HLAA2 MelanA and IMP-2 tetramers; the bar graph (FIG. 35 [lower]) shows flow based killing assay of the IMP2/Melan A T-cell line versus primary autologous CLL cancer cells, (gates were set on single lymphocytes and live CD8+CD3+ cells. The percentage of tetramer+ T-cells is shown for each gate). The data shows that T-cells primed with IMP2 peptide for 2 weeks had IMP2 (8.7%) and Melan A (8.4%) populations, which killed autologous cancer cells.

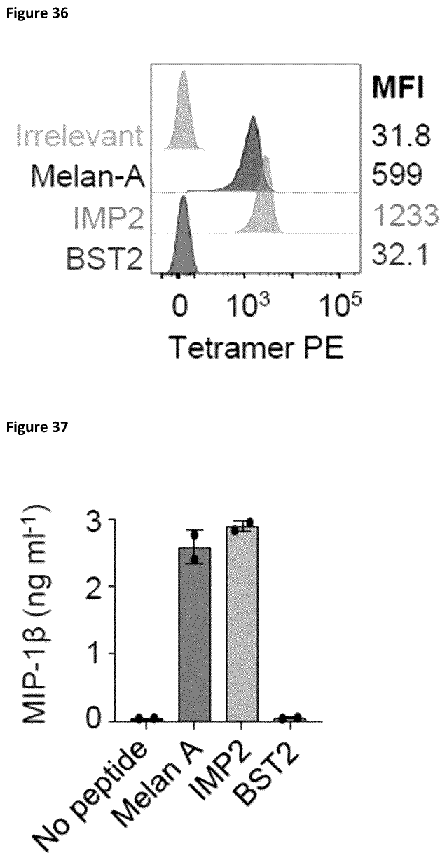

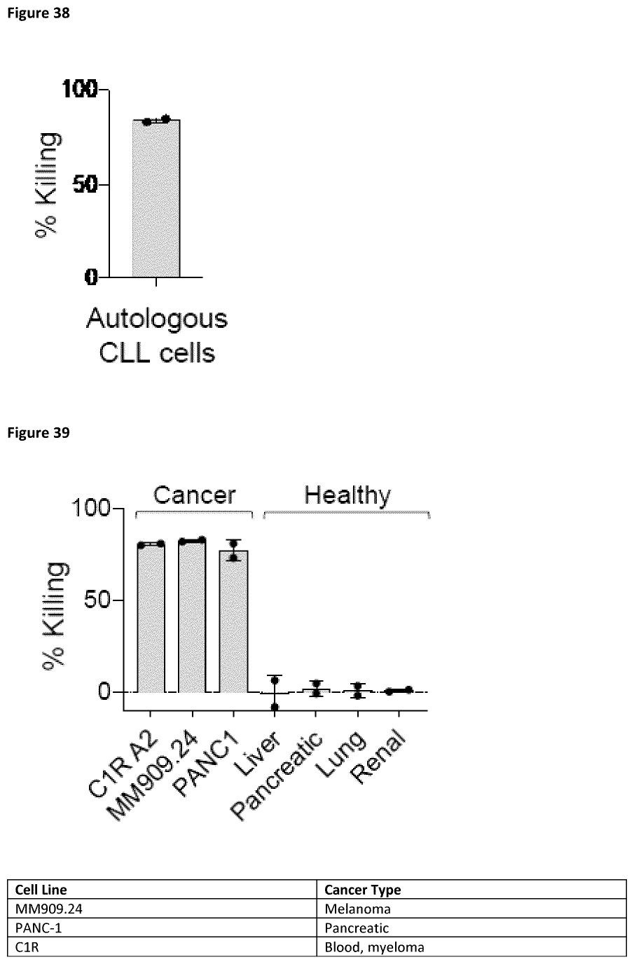

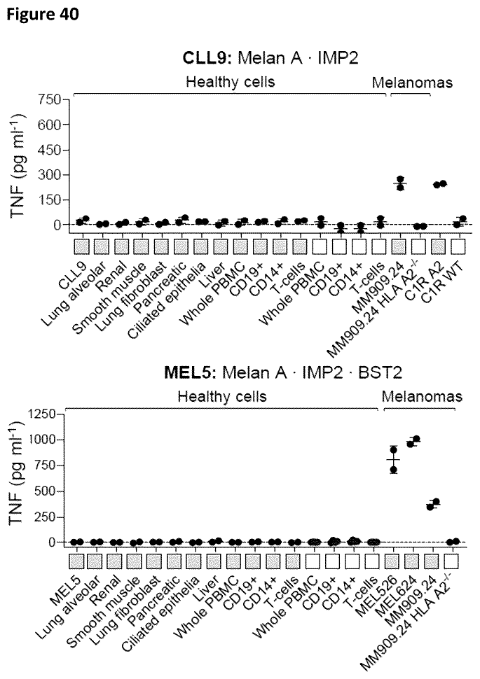

Monoclonal T-cells (T-cell clone CLL9) were produced from the patient U368 T-cell line. TCR sequencing of U368-derived T-cell clone CLL9 revealed TRAV12-2 usage, as also seen for MEL5 and MEL8 (Table 3). CLL9 stained with IMP2 and Melan A tetramers, but not for BST2 (FIG. 36), which was confirmed in a peptide activation assay (FIG. 37). CLL9 killed autologous CLL cells (FIG. 37) and a variety of cancer cell lines (FIG. 38) while remaining inert to healthy cells (FIG. 39). The tri-specific T-cell clone MEL5 and the bi-specific T-cell clone CLL9 have both been shown to killed HLA A*02:01⁺ cancer lines but are demonstrably inert to a wide range of HLA A*02:01⁺ primary cell (FIG. 40). In conclusion, patient T-cells with specificity for Melan A, BST2 and IMP2 or Melan A and IMP2 can kill cancer cell types other than melanoma, including patient autologous primary AML and CLL cells. Further T-cells were isolated in similar fashion from TIL product MM909.24 and were characterized by sequence and target specificity as shown in Table 3 below.

TABLE 3
Summary of isolated multiprong TCR-T cell clones from TIL product
MM909.24 including physical characterization.

| | | CDR1 | CDR2 | | | CDR1 | CDR2 | | |
|---|---|---|---|---|---|---|---|---|---|
| TCR | TRAV | alpha | alpha | CDR3 alpha | TRBV | beta | beta | CDR3 beta | Target |
| MEL54 | 12-2 | DRGSQS | IYSNGD | CAVNNARLMF | 4-2 | LGHNA | YNEKEQ | CASSQGVIVIAGVG | MELA/ |
| | | SEQ ID | SEQ ID | SEQ ID | | SEQ ID | SEQ ID | EQY | BST2/ |
| | | NO: 82 | NO: 83 | NO: 3 | | NO: 90 | NO: 104 | SEQ ID NO: 124 | IMP2 |
| MEL55 | 12-2 | DRGSQS | IYSNGD | CAVNQGGKLIF | 6-1 | MNHNS | SASEGT | CASSEAVLYVDTQY | MELA/ |
| | | SEQ ID | SEQ ID | SEQ ID NO: | | SEQ ID | SEQ ID | F | BST2 |
| | | NO: 82 | NO: 83 | 13 | | NO: 93 | NO: 109 | SEQ ID NO: 125 | |
| MEL56 | 12-3 | NSAFQY | TYSSGN | CAMSVGFGNV | 27 | MNHEY | SMNVEV | CASSLGLAGNEQYF | MELA/ |
| | | SEQ ID | SEQ ID | LHC | | SEQ ID | SEQ ID | SEQ ID NO: 54 | IMP2 |
| | | NO: 131 | NO: 132 | SEQ ID NO: | | NO: 84 | NO: 98 | | |
| | | | | 19 | | | | | |
| MEL57 | 12-2 | DRGSQS | IYSNGD | CAVSIGFGNV | 27 | MNHEY | SMNVEV | CASSFAGTDTQYF | MELA/ |
| | | SEQ ID | SEQ ID | LHC | | SEQ ID | SEQ ID | SEQ ID NO: 126 | IMP2 |
| | | NO: 82 | NO: 83 | SEQ ID NO: | | NO: 84 | NO: 98 | | |
| | | | | 15 | | | | | |
| MEL58 | 23DV6 | NTAFDY | IRPDVSE | CAARGAQKLVF | 6-5 | MNHEY | SVGAGI | CASSYVGLGSPLHE | MELA/ |
| | | SEQ ID | SEQ ID | SEQ ID NO: | | SEQ ID | SEQ ID | SEQ ID NO: 60 | IMP2 |
| | | NO: 133 | NO: 134 | 21 | | NO: 84 | NO: 97 | | |

### Sequences

TABLE 4

| CDR3-alpha | SEQ ID NO | TRAV | TRAJ | TCR SPECIFICTY |
|---|---|---|---|---|
| CAVQKLVF | SEQ ID NO: 1 | TRAV12-2 | TRAJ8 | MelA/BST2/IMP2 |
| CAAAGGGADGLT | SEQ ID NO: 2 | TRAV12-2 | TRAJ45 | MelA/BST2/IMP2 |
| F | | | | |
| CAVNNARLMF | SEQ ID NO: 3 | TRAV12-2 | TRAJ31 | MelA/BST2/IMP2 |
| CAVNTGGFKTIF | SEQ ID NO: 4 | TRAV12-2 | TRAJ9 | MelA/BST2/IMP2 |
| CAAGGGADGLTF | SEQ ID NO: 5 | TRAV12-2 | TRAJ45 | MelA/BST2/IMP2 |
| CALGFGNVLHC | SEQ ID NO: 6 | TRAV12-2 | TRAJ35 | MelA/BST2/IMP2 |
| CAVNIGARLMF | SEQ ID NO: 7 | TRAV12-2 | TRAJ31 | BST2/IMP2 |
| CAVASDGQKLLF | SEQ ID NO: 8 | TRAV12-2 | TRAJ16 | BST2/IMP2 |
| CAVYGGSQGNLIF | SEQ ID NO: 9 | TRAV12-2 | TRAJ42 | BST2/IMP2 |
| CAVNQFYF | SEQ ID NO: 10 | TRAV12-2 | TRAJ49 | MelA/BST2 |
| CAGGGGADGLTF | SEQ ID NO: 11 | TRAV12-2 | TRAJ45 | MelA/BST2 |
| CAANNARLMF | SEQ ID NO: 12 | TRAV12-2 | TRAJ31 | MelA/BST2 |
| CAVNQGGKLIF | SEQ ID NO: 13 | TRAV12-2 | TRAJ23 | MelA/BST2 |
| CAVNSGGGADGL | SEQ ID NO: 14 | TRAV12-2 | TRAJ45 | MelA/IMP2 |
| TF | | | | |
| CAVSIGFGNVLHC | SEQ ID NO: 15 | TRAV12-2 | TRAJ35 | MelA/IMP2 |
| CAVHTGGFKTIF | SEQ ID NO: 16 | TRAV12-2 | TRAJ9 | MelA/IMP2 |
| CAVDNQGGKLIF | SEQ ID NO: 17 | TRAV12-2 | TRAJ23 | MelA/IMP2 |
| CAVSIGFGNVLHC | SEQ ID NO: 18 | TRAV12-2 | TRAJ35 | MelA/IMP2 |
| CAMSVGFGNVLH | SEQ ID NO: 19 | TRAV12-3 | TRAJ35 | MelA/IMP2 |
| C | | | | |
| CAALYNTDKLIF | SEQ ID NO: 20 | TRAV29DV5 | TRAJ34 | MelA/IMP2 |
| CAARGAQKLVF | SEQ ID NO: 21 | TRAV23DV6 | TRAJ54 | MelA/IMP2 |
| CAATLYNNDMRF | SEQ ID NO: 22 | TRAV13-1 | TRAJ43 | MelA/IMP2 |
| CVVSASNGNKLV | SEQ ID NO: 23 | TRAV10 | TRAJ47 | MelA/IMP2 |
| F | | | | |
| CAYLVGSNYQLIW | SEQ ID NO: 24 | TRAV38-2DV8 | TRAJ33 | MelA/IMP2 |
| CAFMKPNTGNQF | SEQ ID NO: 25 | TRAV38-1 | TRAJ49 | MelA/IMP2 |
| YF | | | | |
| CAVNVAGKSTF | SEQ ID NO: 116 | TRAV12-2 | TRAJ27 | MelA/IMP2/BST2 |
| CAVGAGGFKTIF | SEQ ID NO: 117 | TRAV12-2 | TRAJ9 | MelA/IMP2/BST2 |
| CAVNYEDDKIIF | SEQ ID NO: 118 | TRAV12-2 | TRAJ30 | MelA/IMP2/BST2 |
| CAVSDNGNKLVF | SEQ ID NO: 119 | TRAV8-6 | TRAJ47 | MelA/IMP2/BST2 |
| CGSNTGNQFYF | SEQ ID NO: 120 | TRAV12-2 | TRAJ49 | MelA/IMP2/BST2 |

TABLE 5

| CDR3-beta | SEQ ID NO | TRBV | TRBJ | TCR SPECIFICTY |
|---|---|---|---|---|
| CASSPYSGSGET | SEQ ID NO: 26 | TRBV6-5 | TRBJ2-5 | MelA/BST2/IMP2 |
| QYF | | | | |
| CASSFAGTDTQY | SEQ ID NO: 27 | TRBV27 | TRBJ2-3 | MelA/BST2/IMP2 |
| F | | | | |
| CASSYSFTEATY | SEQ ID NO: 28 | TRBV6-5 | TRBJ2-7 | MelA/BST2/IMP2 |
| EQYF | | | | |
| CASSLTGLGQPQ | SEQ ID NO: 29 | TRBV28 | TRBJ1-5 | MelA/BST2/IMP2 |
| HF | | | | |
| CASSYRGLGQPQ | SEQ ID NO: 30 | TRBV28 | TRBJ1-5 | MelA/BST2/IMP2 |
| HF | | | | |
| CASSATVLGTEA | SEQ ID NO: 31 | TRBV19 | TRBJ1-1 | MelA/BST2/IMP2 |
| FF | | | | |
| CSVEESSGIYEQ | SEQ ID NO: 32 | TRBV29-1 | TRBJ2-7 | MelA/BST2/IMP2 |
| YF | | | | |
| CASSSAGMGQPQ | SEQ ID NO: 33 | TRBV27 | TRBJ1-5 | MelA/BST2/IMP2 |
| HF | | | | |
| CAWSSQGLGQPQ | SEQ ID NO: 34 | TRBV30 | TRBJ1-5 | MelA/BST2/IMP2 |
| HF | | | | |
| CASTLGTGDGYT | SEQ ID NO: 35 | TRBV19 | TRBJ1-2 | MelA/BST2/IMP2 |
| F | | | | |
| CSAPQTGLGQPQ | SEQ ID NO: 36 | TRBV20-1 | TRBJ1-5 | MelA/BST2/IMP2 |
| HF | | | | |
| CASSFGPLANYG | SEQ ID NO: 37 | TRBV19 | TRBJ1-2 | MelA/BST2/IMP2 |
| YTF | | | | |
| CASSQGLAGSNE | SEQ ID NO: 38 | TRBV4-2 | TRBJ2-1 | MelA/BST2/IMP2 |
| QFF | | | | |
| CSATGLAGLGEQ | SEQ ID NO: 39 | TRBV20-1 | TRBJ2-1 | MelA/BST2/IMP2 |
| FF | | | | |
| CASSLTGLGQPQ | SEQ ID NO: 40 | TRBV5-6 | TRBJ1-5 | MelA/BST2/IMP2 |
| HF | | | | |
| CASSPGTIYQPQ | SEQ ID NO: 41 | TRBV4-3 | TRBJ1-5 | MelA/BST2/IMP2 |
| HF | | | | |
| CASAGGALVGYG | SEQ ID NO: 42 | TRBV6-5 | TRBJ1-2 | MelA/BST2/IMP2 |
| YTF | | | | |
| CASSFQGLGQPQ | SEQ ID NO: 43 | TRBV28 | TRBJ1-5 | BST2/IMP2 |
| HF | | | | |
| CASTLGGTLGSP | SEQ ID NO: 44 | TRBV19 | TRBJ1-6 | BST2/IMP2 |
| LHF | | | | |
| CASTLGQGWEQY | SEQ ID NO: 45 | TRBV6-2 | TRBJ2-7 | BST2/IMP2 |
| F | | | | |
| CASSLQGLGQPQ | SEQ ID NO: 46 | TRBV28 | TRBJ1-5 | MelA/BST2 |
| HF | | | | |
| CASSLSVISTGE | SEQ ID NO: 47 | TRBV27 | TRBJ2-2 | MelA/BST2 |
| LFF | | | | |
| CASGKGQIFAGE | SEQ ID NO: 48 | TRBV28 | TRBJ2-2 | MelA/BST2 |
| LFF | | | | |
| CATSGLAGAYEQ | SEQ ID NO: 49 | TRBV7-2 | TRBJ2-7 | MelA/BST2 |
| YF | | | | |
| CASSEYTSGNQP | SEQ ID NO: 50 | TRBV6-1 | TRBJ1-5 | MelA/BST2 |
| QHF | | | | |
| CASSPAGLGQPQ | SEQ ID NO: 51 | TRBV6-2 | TRBJ1-5 | MelA/BST2 |
| HF | | | | |
| CASSPDPFGLAG | SEQ ID NO: 52 | TRBV7-2 | TRBJ2-1 | MelA/BST2 |
| NEQFF | | | | |
| CASSWAGPVEQY | SEQ ID NO: 53 | TRBV12-4 | TRBJ2-7 | MelA/IMP2 |
| F | | | | |
| CASSLGLAGNEQ | SEQ ID NO: 54 | TRBV27 | TRBJ2-7 | MelA/IMP2 |
| YF | | | | |
| CASSSGLTDTQY | SEQ ID NO: 55 | TRBV7-3 | TRBJ2-3 | MelA/IMP2 |
| F | | | | |
| CASSLGIISGQP | SEQ ID NO: 56 | TRBV13 | TRBJ1-5 | MelA/IMP2 |
| QHF | | | | |
| CASSLGGGGYEQ | SEQ ID NO: 57 | TRBV27 | TRBJ2-7 | MelA/IMP2 |
| YF | | | | |
| CAISPGEGTDTQ | SEQ ID NO: 58 | TRBV10-3 | TRBJ2-3 | MelA/IMP2 |
| YF | | | | |
| CASSYTGTLSYE | SEQ ID NO: 59 | TRBV28 | TRBJ2-7 | MelA/IMP2 |
| QYF | | | | |
| CASSYVGLGSPL | SEQ ID NO: 60 | TRBV6-5 | TRBJ1-6 | MelA/IMP2 |
| HF | | | | |
| CATSRDMDPEQY | SEQ ID NO: 61 | TRBV15 | TRBJ2-7 | MelA/IMP2 |
| F | | | | |
| CASSLGGADTQY | SEQ ID NO: 62 | TRBV27 | TRBJ2-3 | MelA/IMP2 |
| F | | | | |
| CASSFLQGTGEL | SEQ ID NO: 63 | TRBV27 | TRBJ2-2 | MelA/IMP2 |
| FF | | | | |
| CASRVGILSGEL | SEQ ID NO: 64 | TRBV27 | TRBJ2-2 | MelA/IMP2 |
| FF | | | | |
| CATSRDRGWEQF | SEQ ID NO: 65 | TRBV15 | TRBJ2-1 | MelA/IMP2 |
| F | | | | |
| CASSLSGAEAFF | SEQ ID NO: 66 | TRBV27 | TRBJ1-1 | MelA/IMP2 |
| CASSPSWAHETQ | SEQ ID NO: 67 | TRBV27 | TRBJ2-5 | MelA/IMP2 |
| YF | | | | |
| CASSPSGTVYEK | SEQ ID NO: 68 | TRBV28 | TRBJ1-4 | MelA/IMP2 |
| LFF | | | | |
| CSASEGIGQPQH | SEQ ID NO: 69 | TRBV20-1 | TRBJ1-5 | MelA/IMP2 |
| F | | | | |
| CASSPGLTANVL | SEQ ID NO: 70 | TRBV27 | TRBJ2-6 | MelA/IMP2 |
| TF | | | | |
| CASSQEQLAGPE | SEQ ID NO: 71 | TRBV4-2 | TRBJ2-7 | MelA/IMP2 |
| QYF | | | | |
| CAWSETGLGTGE | SEQ ID NO: 121 | TRBV30-2 | TRBJ2-7 | MelA/IMP2/BST2 |
| LFF | | | | |
| CASSESPLSGNE | SEQ ID NO: 122 | TRBV2-2 | TRBJ2-7 | MelA/IMP2/BST2 |
| QYF | | | | |
| CASSVGVQGSWE | SEQ ID NO: 123 | TRBV9 | TRBJ2-7 | MelA/IMP2/BST2 |
| QYF | | | | |
| CASSQGVLVIAG | SEQ ID NO: 124 | TRBV4-2 | TRBJ2-7 | MelA/IMP2/BST2 |
| VGEQYF | | | | |
| CASSEAVLYVDT | SEQ ID NO: 125 | TRBV6-1 | TRBJ2-3 | MelA/ BST2 |
| QYF | | | | |
| CASSFAGTDTQY | SEQ ID NO: 126 | TRBV27 | TRBJ2-3 | MelA/IMP2 |
| F | | | | |

TABLE 6

| | | | | | TCR |
|---|---|---|---|---|---|
| CDR3-beta | CDR1-beta | CDR2-beat | TRBV | TRBJ | SPECIFICTY |
| CASSPYSGSGETQYF | MNHEY SEQ | SVGAGI SEQ ID | TRBV6-5 | TRBJ2- | MelA/BST2/IMP2 |
| SEQ ID NO: 26 | ID NO: 84 | NO: 97 | | 5 | |
| CASSFAGTDTQYF | MNHEY SEQ | SMNVEV SEQ | TRBV27 | TRBJ2- | MelA/BST2/IMP2 |
| SEQ ID NO: 27 | ID NO: 84 | ID NO: 98 | | 3 | |
| CASSYSFTEATYEQYR | MNHEY SEQ | SVGAGI SEQ ID | TRBV6-5 | TRBJ2- | MelA/BST2/IMP2 |
| SEQ ID NO: 28 | ID NO: 84 | NO: 97 | | 7 | |
| CASSLTGLGQPQHF | MDHEN SEQ | SYDVKM SEQ | TRBV28 | TRBJ1- | MelA/BST2/IMP2 |
| SEQ ID NO: 29 | ID NO: 85 | ID NO: 99 | | 5 | |
| CASSYRGLGQPQHF | MDHEN SEQ | SYDVKM SEQ | TRBV28 | TRBJ1- | MelA/BST2/IMP2 |
| SEQ ID NO: 30 | ID NO: 85 | ID NO: 99 | | 5 | |
| CASSATVLGTEAFF | LNHDA SEQ | SQIVND SEQ ID | TRBV19 | TRBJ1- | MelA/BST2/IMP2 |
| SEQ ID NO: 31 | ID NO: 86 | NO: 100 | | 1 | |
| CSVEESSGIYEQYF | SQVTM SEQ | ANQGSEA SEQ | TRBV29- | TRBJ2- | MelA/BST2/IMP2 |
| SEQ ID NO: 32 | ID NO: 87 | ID NO: 101 | 1 | 7 | |
| CASSSAGMGQPQHF | MNHEY SEQ | SMNVEV SEQ | TRBV27 | TRBJ1- | MelA/BST2/IMP2 |
| SEQ ID NO: 33 | ID NO: 84 | ID NO: 98 | | 5 | |
| CAWSSQGLGQPQHF | GTSNPN SEQ | SVGIG SEQ ID | TRBV30 | TRBJ1- | MelA/BST2/IMP2 |
| SEQ ID NO: 34 | ID NO: 88 | NO: 102 | | 5 | |
| CASTLGTGDGYTF | LNHDA SEQ | SQIVND SEQ ID | TRBV19 | TRBJ1- | MelA/BST2/IMP2 |
| SEQ ID NO: 35 | ID NO: 86 | NO: 100 | | 2 | |
| CSAPQTGLGQPQHF | DFQATT SEQ | SNEGSKA SEQ | TRBV20- | TRBJ1- | MelA/BST2/IMP2 |
| SEQ ID NO: 36 | ID NO: 89 | ID NO: 103 | 1 | 5 | |
| CASSFGPLANYGYTF | LNHDA SEQ | SQIVND SEQ ID | TRBV19 | TRBJ1- | MelA/BST2/IMP2 |
| SEQ ID NO: 37 | ID NO: 86 | NO: 100 | | 2 | |
| CASSQGLAGSNEQFF | LGHNA SEQ | YNFKEQ SEQ ID | TRBV4-2 | TRBJ2- | MelA/BST2/IMP2 |
| SEQ ID NO: 38 | ID NO: 90 | NO: 104 | | 1 | |
| CSATGLAGLGEQFF | DFQATT SEQ | SNEGSKA SEQ | TRBV20- | TRBJ2- | MelA/BST2/IMP2 |
| SEQ ID NO: 39 | ID NO: 89 | ID NO: 103 | 1 | 1 | |
| CASSLTGLGQPQHF | SGHDT SEQ | YYEEEE SEQ ID | TRBV5-6 | TRBJ1- | MelA/BST2/IMP2 |
| SEQ ID NO: 40 | ID NO: 91 | NO: 105 | | 5 | |
| CASSPGTIYQPQHF | LGHNA SEQ | YSLEER SEQ ID | TRBV4-3 | TRBJ1- | MelA/BST2/IMP2 |
| SEQ ID NO: 41 | ID NO: 90 | NO: 106 | | 5 | |
| CASAGGALVGYGYTF | MNHEY SEQ | SVGAGI SEQ ID | TRBV6-5 | TRBJ1- | MelA/BST2/IMP2 |
| SEQ ID NO: 42 | ID NO: 84 | NO: 97 | | 2 | |
| CASSFQGLGQPQHF | MDHEN SEQ | SYDVKM SEQ | TRBV28 | TRBJ1- | BST2/IMP2 |
| SEQ ID NO: 43 | ID NO: 85 | ID NO: 99 | | 5 | |
| CASTLGGTLGSPLHF | LNHDA SEQ | SQIVND SEQ ID | TRBV19 | TRBJ1- | BST2/IMP2 |
| SEQ ID NO: 44 | ID NO: 86 | NO: 100 | | 6 | |
| CASTLGQGWEQYF | MNHEY SEQ | SVGEGT SEQ ID | TRBV6-2 | TRBJ2- | BST2/IMP2 |
| SEQ ID NO: 45 | ID NO: 84 | NO: 107 | | 7 | |
| CASSLQGLGQPQHF | MDHEN SEQ | SYDVKM SEQ | TRBV28 | TRBJ1- | MelA/BST2 |
| SEQ ID NO: 46 | ID NO: 85 | ID NO: 99 | | 5 | |
| CASSLSVISTGELFF | MNHEY SEQ | SMNVEV SEQ | TRBV27 | TRBJ2- | MelA/BST2 |
| SEQ ID NO: 47 | ID NO: 84 | ID NO: 98 | | 2 | |
| CASGKGQIFAGELFF | MDHEN SEQ | SYDVKM SEQ | TRBV28 | TRBJ2- | MelA/BST2 |
| SEQ ID NO: 48 | ID NO: 85 | ID NO: 99 | | 2 | |
| CATSGLAGAYEQYF | SGHTA SEQ | FQGNSA SEQ | TRBV7-2 | TRBJ2- | MelA/BST2 |
| SEQ ID NO: 49 | ID NO: 92 | ID NO: 108 | | 7 | |
| CASSEYTSGNQPQHF | MNHNS SEQ | SASEGT SEQ ID | TRBV6-1 | TRBJ1- | MelA/BST2 |
| SEQ ID NO: 50 | ID NO: 93 | NO: 109 | | 5 | |
| CASSPAGLGQPQHF | MNHEY SEQ | SVGEGT SEQ ID | TRBV6-2 | TRBJ1- | MelA/BST2 |
| SEQ ID NO: 51 | ID NO: 84 | NO: 107 | | 5 | |
| CASSPDPFGLAGNEQFF | SGHTA SEQ | FQGNSA SEQ | TRBV7-2 | TRBJ2- | MelA/BST2 |
| SEQ ID NO: 52 | ID NO: 92 | ID NO: 108 | | 1 | |
| CASSWAGPVEQYF | SGHDY SEQ | FNNNVP SEQ ID | TRBV12- | TRBJ2- | MelA/IMP2 |
| SEQ ID NO: 53 | ID NO: 94 | NO: 111 | 4 | 7 | |
| CASSLGLAGNEQYF | MNHEY SEQ | SMNVEV SEQ | TRBV27 | TRBJ2- | MelA/IMP2 |
| SEQ ID NO: 54 | ID NO: 84 | ID NO: 98 | | 7 | |
| CASSSGLTDTQYF | SGHTA SEQ | FQGTGA SEQ | TRBV7-3 | TRBJ2- | MelA/IMP2 |
| SEQ ID NO: 55 | ID NO: 92 | ID NO: 112 | | 3 | |
| CASSLGIISGQPQHF | PRHDT SEQ | FYEKMQ SEQ | TRBV13 | TRBJ1- | MelA/IMP2 |
| SEQ ID NO: 56 | ID NO: 95 | ID NO: 113 | | 5 | |
| CASSLGGGGYEQYF | MNHEY SEQ | SMNVEV SEQ | TRBV27 | TRBJ2- | MelA/IMP2 |
| SEQ ID NO: 57 | ID NO: 84 | ID NO: 98 | | 7 | |
| CAISPGEGTDTQYF | ENHRY SEQ | SYGVKD SEQ ID | TRBV10- | TRBJ2- | MelA/IMP2 |
| SEQ ID NO: 58 | ID NO: 110 | NO: 114 | 3 | 3 | |
| CASSYTGTLSYEQYF | MDHEN SEQ | SYDVKM SEQ | TRBV28 | TRBJ2- | MelA/IMP2 |
| SEQ ID NO: 59 | ID NO: 85 | ID NO: 99 | | 7 | |
| CASSYVGLGSPLHF | MNHEY SEQ | SVGAGI SEQ ID | TRBV6-5 | TRBJ1- | MelA/IMP2 |
| SEQ ID NO: 60 | ID NO: 84 | NO: 97 | | 6 | |
| CATSRDMDPEQYF | LNHNV SEQ | YYDKDF SEQ ID | TRBV15 | TRBJ2- | MelA/IMP2 |
| SEQ ID NO: 61 | ID NO: 96 | NO: 115 | | 7 | |
| CASSLGGADTQYF | MNHEY SEQ | SMNVEV SEQ | TRBV27 | TRBJ2- | MelA/IMP2 |
| SEQ ID NO: 62 | ID NO: 84 | ID NO: 98 | | 3 | |
| CASSFLQGTGELFF | MNHEY SEQ | SMNVEV SEQ | TRBV27 | TRBJ2- | MelA/IMP2 |
| SEQ ID NO: 63 | ID NO: 84 | ID NO: 98 | | 2 | |
| CASRVGILSGELFF | MNHEY SEQ | SMNVEV SEQ | TRBV27 | TRBJ2- | MelA/IMP2 |
| SEQ ID NO: 64 | ID NO: 84 | ID NO: 98 | | 2 | |
| CATSRDRGWEQFF | LNHNV SEQ | YYDKDF SEQ ID | TRBV15 | TRBJ2- | MelA/IMP2 |
| SEQ ID NO: 65 | ID NO: 96 | NO: 115 | | 1 | |
| CASSLSGAEAFF | MNHEY SEQ | SMNVEV SEQ | TRBV27 | TRBJ1- | MelA/IMP2 |
| SEQ ID NO: 66 | ID NO: 84 | ID NO: 98 | | 1 | |
| CASSPSWAHETQYF | MNHEY SEQ | SMNVEV SEQ | TRBV27 | TRBJ2- | MelA/IMP2 |
| SEQ ID NO: 67 | ID NO: 84 | ID NO: 98 | | 5 | |
| CASSPSGTVYEKLFF | MDHEN SEQ | SYDVKM SEQ | TRBV28 | TRBJ1- | MelA/IMP2 |
| SEQ ID NO: 68 | ID NO: 85 | ID NO: 99 | | 4 | |
| CSASEGIGQPQHF | DFQATT SEQ | SNEGSKA SEQ | TRBV20- | TRBJ1- | MelA/IMP2 |
| SEQ ID NO: 69 | ID NO: 89 | ID NO: 103 | 1 | 5 | |
| CASSPGLTANVLTF | MNHEY SEQ | SMNVEV SEQ | TRBV27 | TRBJ2- | MelA/IMP2 |
| SEQ ID NO: 70 | ID NO: 84 | ID NO: 98 | | 6 | |
| CASSQEQLAGPEQYF | LGHNA SEQ | YNFKEQ | TRBV4-2 | TRBJ2- | MelA/IMP2 |
| SEQ ID NO: 71 | ID NO: 90 | | | 7 | |
| CAWSETGLGTGELFF | GTSNPN SEQ | SVGIG SEQ ID | TRBV30 | TRBJ2- | MelA/IMP2/BST2 |
| SEQ ID NO: 121 | ID NO: 88 | NO: 102 | | 2 | |
| CASSESPLSGNEQYF | SNHLY SEQ | FYNNEI SEQ ID | TRBV2-2 | TRBJ2- | MelA/IMP2/BST2 |
| SEQ ID NO: 122 | ID NO: 127 | NO: 128 | | 7 | |
| CASSVGVQGSWEQYF | SGDLS SEQ | HYYNGE SEQ | TRBV9 | TRBJ2- | MelA/IMP2/BST2 |
| SEQ ID NO: 123 | ID NO: 129 | ID NO: 130 | | 7 | |
| CASSQGVLVIAGVGEQYF | LGHNA SEQ | YNFKEQ SEQ ID | TRBV4-2 | TRBJ2- | MELA/BST2/IMP2 |
| SEQ ID NO: 124 | ID NO: 90 | NO: 104 | | 7 | |
| CASSEAVLYVDTQYF | MNHNS SEQ | SASEGT SEQ ID | TRBV6-1 | TRBJ2- | MELA/BST2/ |
| SEQ ID NO: 125 | ID NO: 93 | NO: 109 | | 3 | |
| CASSLGLAGNEQYF | MNHEY SEQ | SMNVEV SEQ | TRBV27 | TRBJ2- | MELA/IMP2 |
| SEQ ID NO: 54 | ID NO: 84 | ID NO: 98 | | 3 | |
| CASSFAGTDTQYF | MNHEY SEQ | SMNVEV SEQ | TRBV27 | TRBJ2- | MELA/IMP2 |
| SEQ ID NO: 126 | ID NO: 84 | ID NO: 98 | | 3 | |

Melan A polypeptide
SEQ ID NO: 72
MPREDAHFIY GYPKKGHGHS YTTAEEAAGI GILTVILGVL
LLIGCWYCRR RNGYRALMDK SLHVGTQCAL TRRCPQEGFD
HRDSKVSLQE KNCEPVVPNA PPAYEKLSAE QSPPPYSP.
BST2 polypeptide
SEQ ID NO: 73
MASTSYDYCR VPMEDGDKRC KLLLGIGILV LLIIVILGVP
LIIFTIKANS EACRDGLRAV MECRNVTHLL QQELTEAQKG
FQDVEAQAAT CNHTVMALMA SLDAEKAQGQ KKVEELEGEI
TTLNHKLQDA SAEVERLRRE NQVLSVRIAD KKYYPSSQDS
SSAAAPQLLI VLLGLSALLQ.
IMP2 polypeptide
SEQ ID NO: 74
MMNKLYIGNL SPAVTADDLR QLFGDRKLPL AGQVLLKSGY
AFVDYPDQNW AIRAIETLSG KVELHGKIME VDYSVSKKLR
SRKIQIRNIP PHLQWEVLDG LLAQYGTVEN VEQVNTDTET
AVVNVTYATR EEAKIAMEKL SGHQFENYSF KISYIPDEEV
SSPSPPQRAQ RGDHSSREQG HAPGGTSQAR QIDFPLRILV
PTQFVGAIIG KEGLTIKNIT KQTQSRVDIH RKENSGAAEK
PVTIHATPEG TSEACRMILE IMQKEADETK LAEEIPLKIL
AHNGLVGRLI GKEGRNLKKI EHETGTKITI SSLQDLSIYN
PERTITVKGT VEACASAEIE IMKKLREAFE NDMLAVNQQA
NLIPGLNLSA LGIFSTGLSV LSPPAGPRGA PPAAPYHPFT
THSGYFSSLY PHHQFGPFPH HHSYPEQEIV NLFIPTQAVG
AIIGKKGAHI KQLARFAGAS IKIAPAEGPD VSERMVIITG
PPEAQFKAQG RIFGKLKEEN FFNPKEEVKL EAHIRVPSST
AGRVIGKGGK TVNELQNLTS AEVIVPRDQT PDENEEVIVR
IIGHFFASQT AQRKIREIVQ QVKQQEQKYP QGVASQRSK.
Melan A peptide fragment
SEQ ID NO: 75
EAAGIGILTV.
BST2 peptide fragment
SEQ ID NO: 76
LLLGIGILVL.
IMP2 peptide fragment
SEQ ID NO: 77
NLSALGIFST.
SEQ ID NO: 78
MQKEVEQNSGPLSVPEGAIASLNCTYSDRGSQSFFWYRQYSGKSPELIMS
IYSNGDKEDGRFTAQLNKASQYVSLLIRDSQPSDSATYLCAVQKLVFGTG
TRLLVSP(N)/QNPDPAVYQLRDSKSSDKSVCLFTDFDSQTNVSQSKDSD
VYITDKCVLDMRSMDFKSNSAVAWSNKSDFACANAFNNSIIPEDTFFPSP
ESS. MEL8 alpha chain; variable domain = regular
with CDRs = bold underlined, splice site amino
acid = (), constant domain = italic underlined
SEQ ID NO: 79
MNAGVTQTPKFQVLKTGQSMTLQCAQDMNHEYMSWYRQDPGMGLRLIHYS
VGAGITDQGEVPNGYNVSRSTTEDFPLRLLSAAPSQTSVYFCASSYSFTE
ATYEQYFGPGTRLTVT(E)DLKNVFPPEVAVFEPSEAEISHTQKATLVCL
ATGFYPDHVELSWWVNGKEVHSGVCTDPQPLKEQPALNDSRYALSSRLRV
SATFWQDPRNHFRCQVQFYGLSENDEWTQDRAKPVTQIVSAEAWGRAD.
MEL8 beta chain; variable domain = regular with
CDRs = bold underlined, splice site amino acid =
(), constant domain = italic underlined
SEQ ID NO: 80
atgaaatccttgagagttttactagtgatcctgtggcttcagttgagctg
ggtttggagccaacagaaggaggtggagcagaattctggacccctcagtg
ttccagagggagccattgcctctctcaactgcacttacagtgaccgaggt
tcccagtccttcttctggtacagacaatattctgggaaaagccctgagtt
gataatgtccatatactccaatggtgacaaagaagatggaaggtttacag
cacagctcaataaagccagccagtatgtttctctgctcatcagagactcc
cagcccagtgattcagccacctacctctgtgccgtgcagaaacttgtatt
tggaactggcacccgacttctggtcagtccaaatatccagaaccctgacc
ctgccgtgtaccagctgagagactctaaatccagtgacaagtctgtctgc
ctattcaccgattttgattctcaaacaaatgtgtcacaaagtaaggattc
tgatgtgtatatcacagacaaaactgtgctagacatgaggtctatqgact
tcaagagcaacagtgctgtggcctggagcaacaaatctgactttgcatgt
gcaaacgccttcaacaacagcattattccagaagacaccttcttccccag
cccagaaagttcctqtgatgtcaagctggtcgagaaaagctttgaaacag
atacgaacctaaactttcaaaacctgtcagtgattgggttccgaatcctc
ctcctgaaagtggccgggtttaatctgctcatgacgctgcggctgtggtc
cagc. MEL8 alpha chain native sequence, Leader =
bold, TRAV12-2*02 = regular, CDR3α = bold under-
lined, TRAJ8*01 = italic bold, TRAC1 = italic
underlined.
SEQ ID NO: 81
atgagcatcggcctcctgtgctgtgcagccttgtctctcctgtgggcagg
tccagtgaatgctggtgtcactcagaccccaaaattccaggtcctgaaga
caggacagagcatgacactgcagtgtgcccaggatatgaaccatgaatac
atgtcctggtatcgacaagacccaggcatggggctgaggctgattcatta
ctcagttggtgctggtatcactgaccaaggagaagtccccaatggctaca
atgtctccagatcaaccacagaggatttcccgctcaggctgctgtcggct
gctccctcccagacatctgtgtacttctgtgccagcagttactcctttac
tGAGGcgacctacgagcagtacttcgggccgggcaccaggctcacggtca
cagaggacctgaaaaacgtgttcccacccgaggtcgctgtgtttgagcca
tcagaagcagagatctcccacacccaaaaggccacactggtgtgcctggc
cacaggcttctaccccgaccacgtggagctgagctggtgggtgaatggga
aggaggtgcacagtggggtcagcacagacccgcagcccctcaaggagcag
cccgccctcaatgactccagatactgcctgagcagccgcctgagggtctc
ggccaccttctggcagaacccccgcaaccacttccgctgtcaagtccagt
tctacgggctctcggagaatgacgagtggacccaggatagggccaaacct
gtcacccagatcgtcagcgccgaggcctggggtagagcagactgtggctt
cacctccgagtcttaccagcaaggggtcctgtctgccaccatcctctatg
agatcttgctagggaaggccaccttgtatgccgtgctggtcagtgccctc
gtgctgatggccatggtcaagagaaaggattccagaggc. MEL8 beta
chain native sequence, Leader = bold, TRBV6-5*01 =
regular, CDR3 beta = bold underlined, TRBD2*02 =
capitals, TRBJ2-7*01 = italic bold, TRBC2*01 =
italic underlined,.
MEL8,
SEQ ID NO: 82
DRGSQS(CDR1 alpha TRAV12-2)
MEL8,
SEQ ID NO: 83
IYSNGD(CDR2 alpha TRAV12-2)

1. An isolated anti-cancer T-cell receptor (TCR), or anti-cancer binding fragment thereof, that binds to a plurality of cancer polypeptide antigens or antigenic peptide fragments thereof wherein said cancer polypeptide antigens or antigenic peptide fragments thereof are distinct from each other and are presented at the cell surface of one or more than one type of cancer cell.

2. The isolated anti-cancer T-cell receptor (TCR), or anti-cancer binding fragment thereof according to claim 1 wherein, the cancer polypeptide antigen or antigenic peptide fragment thereof comprises the amino acid sequence motif X-A/G-I/L-G-I-X wherein X is any amino acid, and wherein said isolated anti-cancer T-cell receptor (TCR), or anti-cancer binding fragment thereof binds to an amino acid sequence comprising said sequence motif.

3. The isolated anti-cancer T-cell receptor (TCR), or anti-cancer binding fragment thereof according to claim 1 or 2 wherein, the cancer polypeptide antigen or antigenic peptide fragment thereof comprises an amino acid sequence motif selected from any of;
(a).
X-X-X-A/G-I/L-G-I-X-X-X
(b).
X-X-X-A/G-I/L-G-I-X-X
(c).
X-X-X-A/G-I/L-G-I-X
(d).
X-X-A/G-I/L-G-I-X-X-X
(e).
X-X-A/G-I/L-G-I-X-X
(f).
X-A/G-I/L-G-I-X-X-X
(h).
X-A/G-I/L-G-I-X-X,
or
(i).
X-A/G-I/L-G-I-X;
wherein X is any amino acid, and wherein said isolated anti-cancer T-cell receptor (TCR), or anti-cancer binding fragment thereof binds to an amino acid sequence comprising said sequence motif.

4. The isolated anti-cancer T-cell receptor (TCR), or anti-cancer binding fragment thereof according to any of claims 1 to 3 wherein, the cancer polypeptide antigen is selected from any one or more of Melan-A polypeptide (SEQ ID NO: 72), BST2 polypeptide (SEQ ID NO: 73) and IMP2 polypeptide (SEQ ID NO: 74), or antigenic peptide fragment thereof.

5. The isolated anti-cancer T-cell receptor (TCR), or anti-cancer binding fragment thereof according to claim 4 wherein, the TCR or anti-cancer binding fragment thereof binds to any of;
(a). Melan-A polypeptide (SEQ ID NO: 72), BST2 polypeptide (SEQ ID NO: 73) and IMP2 polypeptide (SEQ ID NO: 74),
(b). Melan-A polypeptide (SEQ ID NO: 72), and BST2 polypeptide (SEQ ID NO: 73,
(c). Melan-A polypeptide (SEQ ID NO: 72) and IMP2 polypeptide (SEQ ID NO: 74), or
(d). BST2 polypeptide (SEQ ID NO: 73) and IMP2 polypeptide (SEQ ID NO: 74); or in in any of (a) to (d) an antigenic peptide fragment thereof respectively.

6. The isolated anti-cancer T-cell receptor (TCR), or anti-cancer binding fragment thereof according to any one of claims 1 to 5 wherein, the antigenic peptide fragment comprises any of 8, 9, 10, 11, 12 consecutive amino acid residues of the cancer polypeptide antigen of any of claims 1 to 5.

7. The isolated anti-cancer T-cell receptor (TCR), or anti-cancer binding fragment thereof according to claim 6 wherein, the antigenic peptide fragment is selected from any one or more of the sequences; (a) EAAGIGILTV (SEQ ID NO: 75, Melan A antigenic peptide fragment), (b) LLLGIGILVL (SEQ ID NO: 76, BST2 antigenic peptide fragment), (c) NLSALGIFST (SEQ ID NO: 77, IMP2 antigenic peptide fragment).

8. The isolated anti-cancer T-cell receptor (TCR), or anti-cancer binding fragment thereof according to claim 7 wherein, the TCR or anti-cancer binding fragment thereof binds to any of;

| | (a). |
|---|---|
| (SEQ ID NO: 75) | |
| | EAAGIGILTV, |
| (SEQ ID NO: 76) | |
| | LLLGIGILVL |
| | and |
| (SEQ ID NO: 77) | |
| | NLSALGIFST, |
| | (b). |
| (SEQ ID NO: 75) | |
| | EAAGIGILTV |
| | and |
| (SEQ ID NO: 76) | |
| | LLLGIGILVL, |
| | (c). |
| (SEQ ID NO: 75) | |
| | EAAGIGILTV |
| | and |
| (SEQ ID NO: 77) | |
| | NLSALGIFST, |
| | or |
| | (d). |
| (SEQ ID NO: 76) | |
| | LLLGIGILVL |
| | and |
| (SEQ ID NO: 77) | |
| | NLSALGIFST. |

9. The isolated anti-cancer T-cell receptor (TCR), or anti-cancer binding fragment thereof according to any one of claims 1 to 8, wherein said antigenic peptide fragment is presented at a cell surface by human leukocyte antigen (HLA) class I molecule and/or wherein the cell is a cancer or tumour cell.

10. The isolated anti-cancer T-cell receptor (TCR) or anti-cancer binding fragment thereof according to any one of claims 1 to 9, wherein the TCR or binding fragment does not comprise an alpha chain variable region comprising a CDR1 alpha comprising the sequence DRGSQS (SEQ ID NO:82), a CDR2 alpha comprising the sequence IYSNGD (SEQ ID NO:83), and a CDR3 alpha comprising the sequence CAVNVAGKSTF (SEQ ID NO:116), and a beta chain variable region comprising a CDR1 beta comprising the sequence (GTSNPN SEQ ID NO:88) a CDR2 beta comprising the sequence SVGIG (SEQ ID NO: 102), a CDR3 beta comprising the sequence CAWSETGLGTGELFF (SEQ ID NO:121).

11. The isolated anti-cancer T-cell receptor (TCR) or anti-cancer binding fragment thereof according to any one of claims 1 to 10, wherein the TCR or binding fragment comprises:
(i) an alpha chain variable region comprising a complementarity-determining region (CDR3 alpha) comprising or consisting of any one of:

| (SEQ ID NO: 1) | |
|---|---|
| | CAVQKLVF, |
| (SEQ ID NO: 2) | |
| | CAAAGGGADGLTF, |
| (SEQ ID NO: 3) | |
| | CAVNNARLMF, |
| (SEQ ID NO: 4) | |
| | CAVNTGGFKTIF, |
| (SEQ ID NO: 5) | |
| | CAAGGGADGLTF, |
| (SEQ ID NO: 6) | |
| | CALGFGNVLHC, |
| (SEQ ID NO: 116) | |
| | CAVNVAGKSTF |
| (SEQ ID NO: 117) | |
| | CAVGAGGFKTIF |
| (SEQ ID NO: 118) | |
| | CAVNYEDDKIIF, |
| | or |
| (SEQ ID NO: 120) | |
| | CGSNTGNQFYF, |

or a variant CDR that has 1, 2, 3, 4 or 5 amino acid variations selected from additions, substitutions and deletions with respect thereto; and/or
(ii) a beta chain variable region comprising a complementarity-determining region (CDR3 beta) comprising or consisting of any one of:

| (SEQ ID NO: 26) | |
|---|---|
| | CASSPYSGSGETQYF, |
| (SEQ ID NO: 27) | |
| | CASSFAGTDTQYF, |
| (SEQ ID NO: 28) | |
| | CASSYSFTEATYEQYF, |
| (SEQ ID NO: 29) | |
| | CASSLTGLGQPQHF, |
| (SEQ ID NO: 30) | |
| | CASSYRGLGQPQHF, |
| (SEQ ID NO: 31) | |
| | CASSATVLGTEAFF, |
| (SEQ ID NO: 32) | |
| | CSVEESSGIYEQYF, |
| (SEQ ID NO: 33) | |
| | CASSSAGMGQPQHF, |
| (SEQ ID NO: 34) | |
| | CAWSSQGLGQPQHF, |
| (SEQ ID NO: 35) | |
| | CASTLGTGDGYTF, |
| (SEQ ID NO: 36) | |
| | CSAPQTGLGQPQHF, |
| (SEQ ID NO: 37) | |
| | CASSFGPLANYGYTF, |
| (SEQ ID NO: 38) | |
| | CASSQGLAGSNEQFF, |
| (SEQ ID NO: 39) | |
| | CSATGLAGLGEQFF, |
| (SEQ ID NO: 40) | |
| | CASSLTGLGQPQHF, |
| (SEQ ID NO: 41) | |
| | CASSPGTIYQPQHF, |
| (SEQ ID NO: 42) | |
| | CASAGGALVGYGYTF, |
| (SEQ ID NO: 121) | |
| | CAWSETGLGTGELFF, |
| (SEQ ID NO: 122) | |
| | CASSESPLSGNEQYF, |
| (SEQ ID NO: 123) | |
| | CASSVGVQGSWEQYF, |
| | or |
| (SEQ ID NO: 124) | |
| | CASSQGVLVIAGVGEQYF; |

or a variant CDR that has 1, 2, 3, 4 or 5 amino acid variations selected from additions, substitutions and deletions with respect thereto, wherein the TCR or binding fragment binds to the cancer polypeptide antigens Melan-A polypeptide (SEQ ID NO: 72), BST2 polypeptide (SEQ ID NO: 73) and IMP2 polypeptide (SEQ ID NO: 74) or antigenic peptide fragments thereof.

12. The isolated anti-cancer T-cell receptor (TCR) or anti-cancer binding fragment thereof according to claim 11 wherein the TCR or binding fragment comprises:
(i) an alpha chain variable region further comprising a complementarity-determining region (CDR1 alpha) comprising the sequence DRGSQS (SEQ ID NO: 82) and a complementarity-determining region (CDR2 alpha) comprising the sequence IYSNGD (SEQ ID NO: 83), or a variant CDR that has 1, 2, or 3 amino acid variations selected from additions, substitutions and deletions with respect thereto; and/or
(ii) a beta chain variable region comprising any one of:
(a). a CDR3 beta sequence comprising the sequence CASSPYSGSGETQYF (SEQ ID NO: 26), and CDR1 beta and CDR2 beta sequence comprising the CDR1 beta and CDR2 beta sequences respectively of TRBV6-5,
(b). a CDR3 beta sequence comprising the sequence CASSFAGTDTQYF (SEQ ID NO: 27), and CDR1 beta and CDR2 beta sequence comprising the CDR1 beta and CDR2 beta sequences respectively of TRBV27,
(c). a CDR3 beta sequence comprising the sequence CASSYSFTEATYEQYF (SEQ ID NO: 28), and CDR1 beta and CDR2 beta sequence comprising the CDR1 beta and CDR2 beta sequences respectively of TRBV6-5,
(d). a CDR3 beta sequence comprising the sequence CASSLTGLGQPQHF (SEQ ID NO: 29), and CDR1 beta and CDR2 beta sequence comprising the CDR1 beta and CDR2 beta sequences respectively of TRBV28,
(e). a CDR3 beta sequence comprising the sequence CASSYRGLGQPQHF (SEQ ID NO: 30), and CDR1 beta and CDR2 beta sequence comprising the CDR1 beta and CDR2 beta sequences respectively of TRBV28,
(f). a CDR3 beta sequence comprising the sequence CASSATVLGTEAFF (SEQ ID NO: 31), and CDR1 beta and CDR2 beta sequence comprising the CDR1 beta and CDR2 beta sequences respectively of TRBV19,
(g). a CDR3 beta sequence comprising the sequence CSVEESSGIYEQYF (SEQ ID NO: 32), and CDR1 beta and CDR2 beta sequence comprising the CDR1 beta and CDR2 beta sequences respectively of TRBV29-1,
(h). a CDR3 beta sequence comprising the sequence CASSSAGMGQPQHF (SEQ ID NO: 33), and CDR1 beta and CDR2 beta sequence comprising the CDR1 beta and CDR2 beta sequences respectively of TRBV27,
(i). a CDR3 beta sequence comprising the sequence CAWSSQGLGQPQHF (SEQ ID NO: 34), and CDR1 beta and CDR2 beta sequence comprising the CDR1 beta and CDR2 beta sequences respectively of TRBV30,
(j). a CDR3 beta sequence comprising the sequence CASTLGTGDGYTF (SEQ ID NO: 35), and CDR1 beta and CDR2 beta sequence comprising the CDR1 beta and CDR2 beta sequences respectively of TRBV19,
(k). a CDR3 beta sequence comprising the sequence CSAPQTGLGQPQHF (SEQ ID NO: 36), and CDR1 beta and CDR2 beta sequence comprising the CDR1 beta and CDR2 beta sequences respectively of TRBV20-1,
(l). a CDR3 beta sequence comprising the sequence CASSFGPLANYGYTF (SEQ ID NO: 37), and CDR1 beta and CDR2 beta sequence comprising the CDR1 beta and CDR2 beta sequences respectively of TRBV19,
(m). a CDR3 beta sequence comprising the sequence CASSQGLAGSNEQFF (SEQ ID NO: 38), and CDR1 beta and CDR2 beta sequence comprising the CDR1 beta and CDR2 beta sequences respectively of TRBV4-2,
(n). a CDR3 beta sequence comprising the sequence CSATGLAGLGEQFF (SEQ ID NO: 39), and CDR1 beta and CDR2 beta sequence comprising the CDR1 beta and CDR2 beta sequences respectively of TRBV20-1,
(o). a CDR3 beta sequence comprising the sequence CASSLTGLGQPQHF (SEQ ID NO: 40), and CDR1 beta and CDR2 beta sequence comprising the CDR1 beta and CDR2 beta sequences respectively of TRBV5-6,
(p). a CDR3 beta sequence comprising the sequence CASSPGTIYQPQHF (SEQ ID NO: 41), and CDR1 beta and CDR2 beta sequence comprising the CDR1 beta and CDR2 beta sequences respectively of TRBV4-3,
(q). a CDR3 beta sequence comprising the sequence CASAGGALVGYGYTF (SEQ ID NO: 42); and CDR1 beta and CDR2 beta sequence comprising the CDR1 beta and CDR2 beta sequences respectively of TRBV6-5,
(r). a CDR3 beta sequence comprising the sequence CAWSETGLGTGELFF (SEQ ID NO: 121) and CDR1 beta and CDR2 beta sequence comprising the CDR1 beta and CDR2 beta sequences respectively of TRBV30-02
(s). a CDR3 beta sequence comprising the sequence CASSESPLSGNEQYF (SEQ ID NO: 122), and CDR1 beta and CDR2 beta sequence comprising the CDR1 beta and CDR2 beta sequences respectively of TRBV2-02,
(t). a CDR3 beta sequence comprising the sequence CASSVGVQGSWEQYF (SEQ ID NO: 123), and CDR1 beta and CDR2 beta sequence comprising the CDR1 beta and CDR2 beta sequences respectively of TRBV9, or
(u). a CDR3 beta sequence comprising the sequence CASSQGVLVIAGVGEQYF (SEQ ID NO:124), and CDR1 beta and CDR2 beta sequence comprising the CDR1 beta and CDR2 beta sequences respectively of TRBV4-2, or a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

13. The isolated anti-cancer T-cell receptor (TCR) or anti-cancer binding fragment thereof according to claim 11 or 12 wherein the TCR or binding fragment comprises:
a beta chain variable region comprising any one of:
(a). a CDR3 beta sequence comprising the sequence CASSPYSGSGETQYF (SEQ ID NO: 26), and CDR1 beta and CDR2 beta sequences comprising the sequences MNHEY (SEQ ID NO:84) and SVGAGI (SEQ ID NO:97) respectively,
(b). a CDR3 beta sequence comprising the sequence CASSFAGTDTQYF (SEQ ID NO: 27), and CDR1 beta and CDR2 beta sequences comprising the sequences MNHEY (SEQ ID NO:84) and SMNVEV (SEQ ID NO:98) respectively,
(c). a CDR3 beta sequence comprising the sequence CASSYSFTEATYEQYF (SEQ ID NO: 28), and CDR1 beta and CDR2 beta sequences comprising the sequences MNHEY (SEQ ID NO:84) and SVGAGI (SEQ ID NO:97) respectively,
(d). a CDR3 beta sequence comprising the sequence CASSLTGLGQPQHF (SEQ ID NO: 29), and CDR1 beta and CDR2 beta sequences comprising the sequences MDHEN (SEQ ID NO:85) and SYDVKM (SEQ ID NO:99) respectively,
(e). a CDR3 beta sequence comprising the sequence CASSYRGLGQPQHF (SEQ ID NO: 30), and CDR1 beta and CDR2 beta sequences comprising the sequences MDHEN (SEQ ID NO:85) and SYDVKM (SEQ ID NO:99) respectively,
(f). a CDR3 beta sequence comprising the sequence CASSATVLGTEAFF (SEQ ID NO: 31), and CDR1 beta and CDR2 beta sequences comprising the sequences LNHDA (SEQ ID NO:86) and SQIVND (SEQ ID NO:100) respectively,
(g). a CDR3 beta sequence comprising the sequence CSVEESSGIYEQYF (SEQ ID NO: 32), and CDR1 beta and CDR2 beta sequences comprising the sequences SQVTM (SEQ ID NO:87) and ANQGSEA (SEQ ID NO:101) respectively,
(h). a CDR3 beta sequence comprising the sequence CASSSAGMGQPQHF (SEQ ID NO: 33), and CDR1 beta and CDR2 beta sequences comprising the sequences MNHEY (SEQ ID NO:84) and SMNVEV (SEQ ID NO:98) respectively,
(i). a CDR3 beta sequence comprising the sequence CAWSSQGLGQPQHF (SEQ ID NO: 34), and CDR1 beta and CDR2 beta sequences comprising the sequences GTSNPN (SEQ ID NO:88) and SVGIG (SEQ ID NO:102) respectively,
(j). a CDR3 beta sequence comprising the sequence CASTLGTGDGYTF (SEQ ID NO: 35), and CDR1 beta and CDR2 beta sequences comprising the sequences LNHDA (SEQ ID NO:86) and SQIVND (SEQ ID NO:100) respectively,
(k). a CDR3 beta sequence comprising the sequence CSAPQTGLGQPQHF (SEQ ID NO: 36), and CDR1 beta and CDR2 beta sequences comprising the sequences DFQATT (SEQ ID NO:89) and SNEGSKA (SEQ ID NO:103) respectively,
(l). a CDR3 beta sequence comprising the sequence CASSFGPLANYGYTF (SEQ ID NO: 37), and CDR1 beta and CDR2 beta sequences comprising the sequences LNHDA (SEQ ID NO:86) and SQIVND (SEQ ID NO:100) respectively,
(m). a CDR3 beta sequence comprising the sequence CASSQGLAGSNEQFF (SEQ ID NO: 38), and CDR1 beta and CDR2 beta sequences comprising the sequences LGHNA (SEQ ID NO:90) and YNFKEQ (SEQ ID NO:104) respectively,
(n). a CDR3 beta sequence comprising the sequence CSATGLAGLGEQFF (SEQ ID NO: 39), and CDR1 beta and CDR2 beta sequences comprising the sequences DFQATT (SEQ ID NO:89) and SNEGSKA (SEQ ID NO:103) respectively,
(o). a CDR3 beta sequence comprising the sequence CASSLTGLGQPQHF (SEQ ID NO: 40), and CDR1 beta and CDR2 beta sequences comprising the sequences SGHDT (SEQ ID NO:91) and YYEEEE (SEQ ID NO:105) respectively,
(p). a CDR3 beta sequence comprising the sequence CASSPGTIYQPQHF (SEQ ID NO: 41), and CDR1 beta and CDR2 beta sequences comprising the sequences LGHNA (SEQ ID NO:90) and YSLEER (SEQ ID NO:106) respectively, or
(q). a CDR3 beta sequence comprising the sequence CASAGGALVGYGYTF (SEQ ID NO: 42); and CDR1 beta and CDR2 beta sequences comprising the sequences MNHEY (SEQ ID NO:84) and SVGAGI (SEQ ID NO:97) respectively,
(r). a CDR3 beta sequence comprising the sequence, CAWSETGLGTGELFF (SEQ ID NO: 121), and CDR1 beta and CDR2 beta sequences comprising the sequences GTSNPN (SEQ ID NO:88) and SVGIG (SEQ ID NO:102), respectively,
(s). a CDR3 beta sequence comprising the sequence CASSESPLSGNEQYF (SEQ ID NO: 122), and CDR1 beta and CDR2 beta sequences comprising the sequences SNHLY (SEQ ID NO: 127) and FYNNEI (SEQ ID NO:128), respectively,
(t). a CDR3 beta sequence comprising the sequence CASSVGVQGSWEQYF (SEQ ID NO: 123), and CDR1 beta and CDR2 beta sequence comprising the sequences SGDLS (SEQ ID NO:129) and HYYNGE (SEQ ID NO:130), respectively,
(u). a CDR3 beta sequence comprising the sequence CASSQGVLVIAGVGEQYF (SEQ ID NO:124), and CDR1 beta and CDR2 beta sequence comprising the sequences LGHNA (SEQ ID NO:90) and YNFKEQ (SEQ ID NO:104), respectively;
or a variant CDR3 beta that has 1, 2, 3, 4 or 5 amino acid variations selected from additions, substitutions and deletions with respect thereto, and/or a variant CDR1 beta or CDR2 beta sequence that has 1, 2, or 3 amino acid variations selected from additions, substitutions and deletions with respect thereto.

14. The isolated anti-cancer T-cell receptor (TCR) or anti-cancer binding fragment thereof according to any one of claims 1 to 10 or 11 to 13, wherein the TCR or binding fragment comprises any of:
(a) an alpha chain variable region comprising a CDR1 alpha comprising the sequence DRGSQS (SEQ ID NO:82), a CDR2 alpha comprising the sequence IYSNGD (SEQ ID NO:83), and a CDR3 alpha comprising the sequence CAVQKLVF (SEQ ID NO:1) and a beta chain variable region comprising a CDR1 beta comprising the sequence MNHEY (SEQ ID NO:84), a CDR2 beta comprising the sequence SVGAGI (SEQ ID NO:97), and a CDR3 beta comprising the sequence CASSYSFTEATYEQYF (SEQ ID NO:28);
(b) an alpha chain variable region comprising a CDR1 alpha comprising the sequence DRGSQS (SEQ ID NO:82), a CDR2 alpha comprising the sequence IYSNGD (SEQ ID NO:83), a CDR3 alpha comprising the sequence CAVGAGGFKTIF (SEQ ID NO:117), and a beta chain variable region comprising a CDR1 beta comprising the sequence SNHLY (SEQ ID NO:127), a CDR2 beta comprising the sequence FYNNEI (SEQ ID NO: 128), a CDR3 beta comprising the sequence CASSESPLSGNEQYF (SEQ ID NO:122); CLL9
(c) an alpha chain variable region comprising a CDR1 alpha comprising the sequence DRGSQS (SEQ ID NO:82), a CDR2 alpha comprising the sequence IYSNGD (SEQ ID NO:83), a CDR3 alpha comprising the sequence CAVNYEDDKIIF (SEQ ID NO:118), and a beta chain variable region comprising a CDR1 beta comprising the sequence SGDLS (SEQ ID NO: 129), a CDR2 beta comprising the sequence HYYNGE (SEQ ID NO:130), a CDR3 beta comprising the sequence CASSWAGPVEQYF (SEQ ID NO:53);
(d) an alpha chain variable region comprising a CDR1 alpha comprising the sequence DRGSQS (SEQ ID NO:82), a CDR2 alpha comprising the sequence IYSNGD (SEQ ID NO:83), a CDR3 alpha comprising the sequence CGSNTGNQFYF (SEQ ID NO:120), and a beta chain variable region comprising a CDR1 beta comprising the sequence GTSNPN (SEQ ID NO:88), a CDR2 beta comprising the sequence SVGIG (SEQ ID NO:102), a CDR3 beta comprising the sequence CAWSSQGLGQPQHF (SEQ ID NO:34); or
(e) an alpha chain variable region comprising a CDR1 alpha comprising the sequence DRGSQS (SEQ ID NO:82), a CDR2 alpha comprising the sequence IYSNGD (SEQ ID NO:83), a CDR3 alpha comprising the sequence CAVNNARLMF (SEQ ID NO:3), and a beta chain variable region comprising a CDR1 beta comprising the sequence LGHNA (SEQ ID NO:90), a CDR2 beta comprising the sequence YNFKEQ (SEQ ID NO:104), a CDR3 beta comprising the sequence CASSQGVLVIAGVGEQYF (SEQ ID NO:124).

15. The isolated anti-cancer T-cell receptor (TCR) or anti-cancer binding fragment thereof according to any one of claims 1 to 10, wherein the TCR or binding fragment comprises:
(i) an alpha chain variable region comprising a complementarity-determining region (CDR3 alpha) comprising or consisting of any one of:

| (SEQ ID NO: 7) | |
|---|---|
| | CAVNIGARLMF |
| (SEQ ID NO: 8) | |
| | CAVASDGQKLLF, |
| | or |
| (SEQ ID NO: 9) | |
| | CAVYGGSQGNLIF; |

or a variant CDR that has 1, 2, 3, 4 or 5 amino acid variations selected from additions, substitutions and deletions with respect thereto; and/or
(ii) a beta chain variable region comprising a complementarity-determining region (CDR3 beta) comprising or consisting of any one of:

| (SEQ ID NO: 43) | |
|---|---|
| | CASSFQGLGQPQHF, |
| (SEQ ID NO: 44) | |
| | CASTLGGTLGSPLHF |
| | or |
| (SEQ ID NO: 45) | |
| | CASTLGQGWEQYF; |

or a variant CDR that has 1, 2, 3, 4 or 5 amino acid variations selected from additions, substitutions and deletions with respect thereto, wherein the TCR or binding fragment binds to the cancer polypeptide antigens BST2 polypeptide (SEQ ID NO: 73) and IMP2 polypeptide (SEQ ID NO: 74) or antigenic peptide fragments thereof.

16. The isolated anti-cancer T-cell receptor (TCR) or anti-cancer binding fragment thereof according to claim 15 wherein the TCR or binding fragment comprises:
(i) an alpha chain variable region further comprising a complementarity-determining region (CDR1 alpha) comprising the sequence DRGSQS (SEQ ID NO: 82) and a complementarity-determining region (CDR2 alpha) comprising the sequence IYSNGD (SEQ ID NO: 83), or a variant CDR that has 1, 2, or 3 amino acid variations selected from additions, substitutions, and deletions with respect thereto; and/or
(ii) a beta chain variable region comprising any one of:
(a). a CDR3 beta sequence comprising the sequence CASSFQGLGQPQHF (SEQ ID NO: 43) and CDR1 beta and CDR2 beta sequence comprising the CDR1 beta and CDR2 beta sequences respectively of TRBV28,
(b). a CDR3 beta sequence comprising the sequence CASTLGGTLGSPLHF (SEQ ID NO: 44) and CDR1 beta and CDR2 beta sequence comprising the CDR1 beta and CDR2 beta sequences respectively of TRBV19, or
(c). a CDR3 beta sequence comprising the sequence CASTLGQGWEQYF (SEQ ID NO: 45) and CDR1 beta and CDR2 beta sequence comprising the CDR1 beta and CDR2 beta sequences respectively of TRBV6-2; or a variant CDR3 beta that has 1, 2, 3, 4 or 5 amino acid variations selected from additions, substitutions and deletions with respect thereto, and/or a variant CDR1 beta or CDR2 beta sequence that has 1, 2, or 3 amino acid variations selected from additions, substitutions and deletions with respect thereto.

17. The isolated anti-cancer T-cell receptor (TCR) or anti-cancer binding fragment thereof according to claim 15 or 16 wherein the TCR or binding fragment comprises:
a beta chain variable region comprising any one of:
(a). a CDR3 beta sequence comprising the sequence CASSFQGLGQPQHF (SEQ ID NO: 43) and CDR1 beta and CDR2 beta sequences comprising the sequences MDHEN (SEQ ID NO:85) and SYDVKM (SEQ ID NO:99) respectively,
(b). a CDR3 beta sequence comprising the sequence CASTLGGTLGSPLHF (SEQ ID NO: 44) and CDR1 beta and CDR2 beta sequences comprising the sequences LNHDA (SEQ ID NO:86) and SQIVND (SEQ ID NO:100) respectively, or
(c). a CDR3 beta sequence comprising the sequence CASTLGQGWEQYF (SEQ ID NO: 45) and CDR1 beta and CDR2 beta sequences comprising the sequences MNHEY (SEQ ID NO:84) and SVGEGT (SEQ ID NO:107) respectively, or a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

18. The isolated anti-cancer T-cell receptor (TCR) or anti-cancer binding fragment thereof according to any one of claims 1 to 10, wherein the TCR or binding fragment comprises:
(i) an alpha chain variable region comprising a complementarity-determining region (CDR3 alpha) comprising or consisting of any one of:

| (SEQ ID NO: 10) | |
|---|---|
| | CAVNQFYF, |
| (SEQ ID NO: 11) | |
| | CAGGGGADGLTF, |
| (SEQ ID NO: 12) | |
| | CAANNARLMF, |
| | or |
| (SEQ ID NO: 13) | |
| | CAVNQGGKLIF; |

or a variant CDR that has 1, 2, 3, 4 or 5 amino acid variations selected from additions, substitutions and deletions with respect thereto; and/or
(ii) a beta chain variable region comprising a complementarity-determining region (CDR3 beta) comprising or consisting of any one of:

| (SEQ ID NO: 46) | |
|---|---|
| | CASSLQGLGQPQHF, |
| (SEQ ID NO: 47) | |
| | CASSLSVISTGELFF, |
| (SEQ ID NO: 48) | |
| | CASGKGQIFAGELFF, |
| (SEQ ID NO: 49) | |
| | CATSGLAGAYEQYF, |
| (SEQ ID NO: 50) | |
| | CASSEYTSGNQPQHF, |
| (SEQ ID NO: 51) | |
| | CASSPAGLGQPQHF, |
| (SEQ ID NO: 52) | |
| | CASSPDPFGLAGNEQFF, |
| | or |
| (SEQ ID NO: 125) | |
| | CASSEAVLYVDTQYF, |

or a variant CDR that has 1, 2, 3, 4 or 5 amino acid variations selected from additions, substitutions, and deletions with respect thereto, wherein the TCR or binding fragment binds to the cancer polypeptide antigens Melan-A polypeptide (SEQ ID NO: 72) and BST2 polypeptide (SEQ ID NO: 73) or antigenic peptide fragments thereof.

19. The isolated anti-cancer T-cell receptor (TCR) or anti-cancer binding fragment thereof according to claim 18 wherein the TCR or binding fragment comprises:
(i) an alpha chain variable region further comprising a complementarity-determining region (CDR1 alpha) comprising the sequence DRGSQS (SEQ ID NO: 82) and a complementarity-determining region (CDR2 alpha) comprising the sequence IYSNGD (SEQ ID NO: 83), or a variant CDR that has 1, 2, or 3 amino acid variations selected from additions, substitutions and deletions with respect thereto; and/or
(ii) a beta chain variable region comprising any one of:
(a). a CDR3 beta sequence comprising the sequence CASSLQGLGQPQHF (SEQ ID NO: 46) and CDR1 beta and CDR2 beta sequence comprising the CDR1 beta and CDR2 beta sequences respectively of TRBV28
(b). a CDR3 beta sequence comprising the sequence CASSLSVISTGELFF (SEQ ID NO: 47) and CDR1 beta and CDR2 beta sequence comprising the CDR1 beta and CDR2 beta sequences respectively of TRBV27
(c). a CDR3 beta sequence comprising the sequence CASGKGQIFAGELFF (SEQ ID NO: 48) and CDR1 beta and CDR2 beta sequence comprising the CDR1 beta and CDR2 beta sequences respectively of TRBV28
(d). a CDR3 beta sequence comprising the sequence CATSGLAGAYEQYF (SEQ ID NO: 49) and CDR1 beta and CDR2 beta sequence comprising the CDR1 beta and CDR2 beta sequences respectively of TRBV7-2
(e). a CDR3 beta sequence comprising the sequence CASSEYTSGNQPQHF (SEQ ID NO: 50) and CDR1 beta and CDR2 beta sequence comprising the CDR1 beta and CDR2 beta sequences respectively of TRBV6-1
(f). a CDR3 beta sequence comprising the sequence CASSPAGLGQPQHF (SEQ ID NO: 51) and CDR1 beta and CDR2 beta sequence comprising the CDR1 beta and CDR2 beta sequences respectively of TRBV6-2,
(g). a CDR3 beta sequence comprising the sequence CASSPDPFGLAGNEQFF (SEQ ID NO:52) and CDR1 beta and CDR2 beta sequence comprising the CDR1 beta and CDR2 beta sequences respectively of TRBV7-2, or
(h). a CDR3 beta sequence comprising the sequence CASSEAVLYVDTQYF (SEQ ID NO: 125) and CDR1 beta and CDR2 beta sequence comprising the CDR1 beta and CDR2 beta sequences respectively of TRBV6-1, or a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

20. The isolated anti-cancer T-cell receptor (TCR) or anti-cancer binding fragment thereof according to claim 18 or 19 wherein the TCR or binding fragment comprises a beta chain variable region comprising any one of:
(a). a CDR3 beta sequence comprising the sequence CASSLQGLGQPQHF (SEQ ID NO: 46) and CDR1 beta and CDR2 beta sequences comprising the sequences MDHEN (SEQ ID NO:85) and SYDVKM (SEQ ID NO:99) respectively,
(b). a CDR3 beta sequence comprising the sequence CASSLSVISTGELFF (SEQ ID NO: 47) and CDR1 beta and CDR2 beta sequences comprising the sequences MNHEY (SEQ ID NO:84) and SMNVEV (SEQ ID NO:98) respectively,
(c). a CDR3 beta sequence comprising the sequence CASGKGQIFAGELFF (SEQ ID NO: 48) and CDR1 beta and CDR2 beta sequences comprising the sequences MDHEN (SEQ ID NO:85) and SYDVKM (SEQ ID NO:99) respectively,
(d). a CDR3 beta sequence comprising the sequence CATSGLAGAYEQYF (SEQ ID NO: 49) and CDR1 beta and CDR2 beta sequences comprising the sequences SGHTA (SEQ ID NO:92) and FQGNSA (SEQ ID NO:108) respectively,
(e). a CDR3 beta sequence comprising the sequence CASSEYTSGNQPQHF (SEQ ID NO: 50) and CDR1 beta and CDR2 beta sequences comprising the sequences MNHNS (SEQ ID NO:93) and SASEGT (SEQ ID NO:109) respectively,
(f). a CDR3 beta sequence comprising the sequence CASSPAGLGQPQHF (SEQ ID NO: 51) and CDR1 beta and CDR2 beta sequences comprising the sequences MNHEY (SEQ ID NO:84) and SVGEGT (SEQ ID NO:107) respectively, or
(g). a CDR3 beta sequence comprising the sequence CASSPDPFGLAGNEQFF (SEQ ID NO:52) and CDR1 beta and CDR2 beta sequences comprising the sequences SGHTA (SEQ ID NO:92) and FQGNSA (SEQ ID NO:108) respectively; or
(h). a CDR3 beta sequence comprising the sequence CASSEAVLYVDTQYF (SEQ ID NO: 125) and CDR1 beta and CDR2 beta sequences comprising the sequences MNHNS (SEQ ID NO:93) and SASEGT (SEQ ID NO: 109) respectively, or a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

21. The isolated anti-cancer T-cell receptor (TCR) or anti-cancer binding fragment thereof according to any one of claims 1 to 10 or 18 to 20, wherein the TCR or binding fragment comprises:
(a) an alpha chain variable region comprising a CDR1 alpha comprising the sequence DRGSQS (SEQ ID NO:82), a CDR2 alpha comprising the sequence IYSNGD (SEQ ID NO:83), and a CDR3 alpha comprising the sequence CAVNQGGKLIF (SEQ ID NO:13) and a beta chain variable region comprising a CDR1 beta comprising the sequence MNHNS (SEQ ID NO:93), a CDR2 beta comprising the sequence SASEGT (SEQ ID NO:109), and a CDR3 beta comprising the sequence CASSEAVLYVDTQYF (SEQ ID NO: 125).

22. The isolated anti-cancer T-cell receptor (TCR) or anti-cancer binding fragment thereof according to any one of claims 1 to 10, wherein the TCR or binding fragment comprises:
(i) an alpha chain variable region comprising a complementarity-determining region (CDR3 alpha) comprising or consisting of any one of:

| (SEQ ID NO: 14) | |
|---|---|
| | CAVNSGGGADGLTF, |
| (SEQ ID NO: 15) | |
| | CAVSIGFGNVLHC, |
| (SEQ ID NO: 16) | |
| | CAVHTGGFKTIF, |
| (SEQ ID NO: 17) | |
| | CAVDNQGGKLIF, |
| | or |
| (SEQ ID NO: 18) | |
| | CAVSIGFGNVLHC; |

or a variant CDR that has 1, 2, 3, 4 or 5 amino acid variations selected from additions, substitutions and deletions with respect thereto; and/or
(ii) a beta chain variable region comprising a complementarity-determining region (CDR3 beta) comprising or consisting of any one of:

| (SEQ ID NO: 53) | |
|---|---|
| | CASSWAGPVEQYF, |
| (SEQ ID NO: 54) | |
| | CASSLGLAGNEQYF, |
| (SEQ ID NO: 55) | |
| | CASSSGLTDTQYF, |
| (SEQ ID NO: 56) | |
| | CASSLGIISGQPQHF, |
| (SEQ ID NO: 57) | |
| | CASSLGGGGYEQYF, |
| (SEQ ID NO: 58) | |
| | CAISPGEGTDTQYF, |
| (SEQ ID NO: 59) | |
| | CASSYTGTLSYEQYF, |
| (SEQ ID NO: 60) | |
| | CASSYVGLGSPLHF, |
| (SEQ ID NO: 61) | |
| | CATSRDMDPEQYF, |
| (SEQ ID NO: 62) | |
| | CASSLGGADTQYF, |
| (SEQ ID NO: 63) | |
| | CASSFLQGTGELFF, |
| (SEQ ID NO: 64) | |
| | CASRVGILSGELFF, |
| (SEQ ID NO: 65) | |
| | CATSRDRGWEQFF, |
| (SEQ ID NO: 66) | |
| | CASSLSGAEAFF, |
| (SEQ ID NO: 67) | |
| | CASSPSWAHETQYF, |
| (SEQ ID NO: 68) | |
| | CASSPSGTVYEKLFF, |
| (SEQ ID NO: 69) | |
| | CSASEGIGQPQHF, |
| (SEQ ID NO: 70) | |
| | CASSPGLTANVLTF, |
| (SEQ ID NO: 71) | |
| | CASSQEQLAGPEQYF, |
| | or |
| (SEQ ID NO: 126) | |
| | CASSFAGTDTQYF, |

or a variant CDR that has 1, 2, 3, 4 or 5 amino acid variations selected from additions, substitutions, and deletions with respect thereto, wherein the TCR or binding fragment binds to the cancer polypeptide antigens Melan-A polypeptide (SEQ ID NO: 72) and IMP2 polypeptide (SEQ ID NO: 74) or antigenic peptide fragments thereof.

23. The isolated anti-cancer T-cell receptor (TCR) or anti-cancer binding fragment thereof according to claim 22 wherein the TCR or binding fragment comprises:
(i) an alpha chain variable region further comprising a complementarity-determining region (CDR1 alpha) comprising the sequence DRGSQS (SEQ ID NO: 82) and a complementarity-determining region (CDR2 alpha) comprising the sequence IYSNGD (SEQ ID NO: 83), or a variant CDR that has 1, 2, or 3 amino acid variations selected from additions, substitutions and deletions with respect thereto; and/or
(ii) a beta chain variable region comprising any one of:
(a). a CDR3 beta sequence comprising the sequence CASSWAGPVEQYF (SEQ ID NO: 53) and CDR1 beta and CDR2 beta sequence comprising the CDR1 beta and CDR2 beta sequences respectively of TRBV12-4,
(b). a CDR3 beta sequence comprising the sequence CASSLGLAGNEQYF (SEQ ID NO: 54) and CDR1 beta and CDR2 beta sequence comprising the CDR1 beta and CDR2 beta sequences respectively of TRBV27,
(c). a CDR3 beta sequence comprising the sequence CASSSGLTDTQYF (SEQ ID NO: 55) TRBV7-3 and CDR1 beta and CDR2 beta sequence comprising the CDR1 beta and CDR2 beta sequences respectively of TRBV7-3,
(d). a CDR3 beta sequence comprising the sequence CASSLGIISGQPQHF (SEQ ID NO: 56) and CDR1 beta and CDR2 beta sequence comprising the CDR1 beta and CDR2 beta sequences respectively of TRBV13,
(e). a CDR3 beta sequence comprising the sequence CASSLGGGGYEQYF (SEQ ID NO: 57) and CDR1 beta and CDR2 beta sequence comprising the CDR1 beta and CDR2 beta sequences respectively of TRBV27,
(f). a CDR3 beta sequence comprising the sequence CAISPGEGTDTQYF (SEQ ID NO: 58) and CDR1 beta and CDR2 beta sequence comprising the CDR1 beta and CDR2 beta sequences respectively of TRBV10-3,
(g). a CDR3 beta sequence comprising the sequence CASSYTGTLSYEQYF (SEQ ID NO: 59) and CDR1 beta and CDR2 beta sequence comprising the CDR1 beta and CDR2 beta sequences respectively of TRBV28,
(h). a CDR3 beta sequence comprising the sequence CASSYVGLGSPLHF (SEQ ID NO: 60) and CDR1 beta and CDR2 beta sequence comprising the CDR1 beta and CDR2 beta sequences respectively of TRBV6-5,
(i). a CDR3 beta sequence comprising the sequence CATSRDMDPEQYF (SEQ ID NO: 61) and CDR1 beta and CDR2 beta sequence comprising the CDR1 beta and CDR2 beta sequences respectively of TRBV15,
(j). a CDR3 beta sequence comprising the sequence CASSLGGADTQYF (SEQ ID NO: 62) and CDR1 beta and CDR2 beta sequence comprising the CDR1 beta and CDR2 beta sequences respectively of TRBV27,
(k). a CDR3 beta sequence comprising the sequence CASSFLQGTGELFF (SEQ ID NO: 63) and CDR1 beta and CDR2 beta sequence comprising the CDR1 beta and CDR2 beta sequences respectively of TRBV27,
(l). a CDR3 beta sequence comprising the sequence CASRVGILSGELFF (SEQ ID NO: 64) and CDR1 beta and CDR2 beta sequence comprising the CDR1 beta and CDR2 beta sequences respectively of TRBV27,
(m). a CDR3 beta sequence comprising the sequence CATSRDRGWEQFF (SEQ ID NO: 65) and CDR1 beta and CDR2 beta sequence comprising the CDR1 beta and CDR2 beta sequences respectively of TRBV15,
(n). a CDR3 beta sequence comprising the sequence CASSLSGAEAFF (SEQ ID NO: 66) and CDR1 beta and CDR2 beta sequence comprising the CDR1 beta and CDR2 beta sequences respectively of TRBV27,
(o). a CDR3 beta sequence comprising the sequence CASSPSWAHETQYF (SEQ ID NO: 67) and CDR1 beta and CDR2 beta sequence comprising the CDR1 beta and CDR2 beta sequences respectively of TRBV27,
(p). a CDR3 beta sequence comprising the sequence CASSPSGTVYEKLFF (SEQ ID NO: 68) and CDR1 beta and CDR2 beta sequence comprising the CDR1 beta and CDR2 beta sequences respectively of TRBV28,
(q). a CDR3 beta sequence comprising the sequence CSASEGIGQPQHF (SEQ ID NO: 69) and CDR1 beta and CDR2 beta sequence comprising the CDR1 beta and CDR2 beta sequences respectively of TRBV20-1,
(r). a CDR3 beta sequence comprising the sequence CASSPGLTANVLTF (SEQ ID NO: 70) and CDR1 beta and CDR2 beta sequence comprising the CDR1 beta and CDR2 beta sequences respectively of TRBV27,
(s). a CDR3 beta sequence comprising the sequence CASSQEQLAGPEQYF (SEQ ID NO: 71) and CDR1 beta and CDR2 beta sequence comprising the CDR1 beta and CDR2 beta sequences respectively of TRBV4-2; or
(t). a CDR3 beta sequence comprising the sequence CASSFAGTDTQYF (SEQ ID NO: 126) and CDR1 beta and CDR2 beta sequence comprising the CDR1 beta and CDR2 beta sequences respectively of TRBV27; or a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

24. The isolated anti-cancer T-cell receptor (TCR) or anti-cancer binding fragment thereof according to claim 22 or 23 wherein the TCR or binding fragment comprises a beta chain variable region comprising any one of:
(a). a CDR3 beta sequence comprising the sequence CASSWAGPVEQYF (SEQ ID NO: 53) and CDR1 beta and CDR2 beta sequences comprising the sequences SGHDY (SEQ ID NO:94) and FNNNVP (SEQ ID NO:111) respectively,
(b). a CDR3 beta sequence comprising the sequence CASSLGLAGNEQYF (SEQ ID NO: 54) and CDR1 beta and CDR2 beta sequences comprising the sequences MNHEY (SEQ ID NO:84) and SMNVEV (SEQ ID NO:98) respectively,
(c). a CDR3 beta sequence comprising the sequence CASSSGLTDTQYF (SEQ ID NO: 55), and CDR1 beta and CDR2 beta sequences comprising the sequences SGHTA (SEQ ID NO:92) and FQGTGA (SEQ ID NO:112) respectively,
(d). a CDR3 beta sequence comprising the sequence CASSLGIISGQPQHF (SEQ ID NO: 56) and CDR1 beta and CDR2 beta sequences comprising the sequences PRHDT (SEQ ID NO:95) and FYEKMQ (SEQ ID NO:113) respectively,
(e). a CDR3 beta sequence comprising the sequence CASSLGGGGYEQYF (SEQ ID NO: 57) and CDR1 beta and CDR2 beta sequences comprising the sequences MNHEY (SEQ ID NO:84) and SMNVEV (SEQ ID NO:98) respectively,
(f). a CDR3 beta sequence comprising the sequence CAISPGEGTDTQYF (SEQ ID NO: 58) and CDR1 beta and CDR2 beta sequences comprising the sequences ENHRY (SEQ ID NO:110) and SYGVKD (SEQ ID NO:114) respectively,
(g). a CDR3 beta sequence comprising the sequence CASSYTGTLSYEQYF (SEQ ID NO: 59) and CDR1 beta and CDR2 beta sequences comprising the sequences MDHEN (SEQ ID NO:85) and SYDVKM (SEQ ID NO:99) respectively,
(h). a CDR3 beta sequence comprising the sequence CASSYVGLGSPLHF (SEQ ID NO: 60) and CDR1 beta and CDR2 beta sequences comprising the sequences MNHEY (SEQ ID NO:84) and SVGAGI (SEQ ID NO:97) respectively,
(i). a CDR3 beta sequence comprising the sequence CATSRDMDPEQYF (SEQ ID NO: 61) and CDR1 beta and CDR2 beta sequences comprising the sequences LNHNV (SEQ ID NO:96) and YYDKDF (SEQ ID NO:115) respectively,
(j). a CDR3 beta sequence comprising the sequence CASSLGGADTQYF (SEQ ID NO: 62) and CDR1 beta and CDR2 beta sequences comprising the sequences MNHEY (SEQ ID NO:84) and SMNVEV (SEQ ID NO:98) respectively,
(k). a CDR3 beta sequence comprising the sequence CASSFLQGTGELFF (SEQ ID NO: 63) and CDR1 beta and CDR2 beta sequences comprising the sequences MNHEY (SEQ ID NO:84) and SMNVEV (SEQ ID NO:98) respectively,
(l). a CDR3 beta sequence comprising the sequence CASRVGILSGELFF (SEQ ID NO: 64) and CDR1 beta and CDR2 beta sequences comprising the sequences MNHEY (SEQ ID NO:84) and SMNVEV (SEQ ID NO:98) respectively,
(m). a CDR3 beta sequence comprising the sequence CATSRDRGWEQFF (SEQ ID NO: 65) and CDR1 beta and CDR2 beta sequences comprising the sequences LNHNV (SEQ ID NO:96) and YYDKDF (SEQ ID NO:115) respectively,
(n). a CDR3 beta sequence comprising the sequence CASSLSGAEAFF (SEQ ID NO: 66) and CDR1 beta and CDR2 beta sequences comprising the sequences MNHEY (SEQ ID NO:84) and SMNVEV (SEQ ID NO:98) respectively,
(o). a CDR3 beta sequence comprising the sequence CASSPSWAHETQYF (SEQ ID NO: 67) and CDR1 beta and CDR2 beta sequences comprising the sequences MNHEY (SEQ ID NO:84) and SMNVEV (SEQ ID NO:98) respectively,
(p). a CDR3 beta sequence comprising the sequence CASSPSGTVYEKLFF (SEQ ID NO: 68) and CDR1 beta and CDR2 beta sequences comprising the sequences MDHEN (SEQ ID NO:85) and SYDVKM (SEQ ID NO:99) respectively,
(q). a CDR3 beta sequence comprising the sequence CSASEGIGQPQHF (SEQ ID NO: 69) and CDR1 beta and CDR2 beta sequences comprising the sequences DFQATT (SEQ ID NO:89) and SNEGSKA (SEQ ID NO:103) respectively,
(r). a CDR3 beta sequence comprising the sequence CASSPGLTANVLTF (SEQ ID NO: 70) and CDR1 beta and CDR2 beta sequences comprising the sequences MNHEY (SEQ ID NO:84) and SMNVEV (SEQ ID NO:98) respectively,
(s). a CDR3 beta sequence comprising the sequence CASSQEQLAGPEQYF (SEQ ID NO: 71) and CDR1 beta and CDR2 beta sequences comprising the sequences LGHNA (SEQ ID NO:90) and YNFKEQ (SEQ ID NO:104) respectively; or
(s). a CDR3 beta sequence comprising the sequence CASSFAGTDTQYF (SEQ ID NO: 126) and CDR1 beta and CDR2 beta sequences comprising the sequences MNHEY (SEQ ID NO:84) and SMNVEV (SEQ ID NO:98), respectively; or a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

25. The isolated anti-cancer T-cell receptor (TCR) or anti-cancer binding fragment thereof according to any one of claims 1 to 10 or 22 to 24, wherein the TCR or binding fragment comprises: (a) an alpha chain variable region comprising a CDR1 alpha comprising the sequence DRGSQS (SEQ ID NO:82), a CDR2 alpha comprising the sequence IYSNGD (SEQ ID NO:83), and a CDR3 alpha comprising the sequence CAVSIGFGNVLHC (SEQ ID NO: 15) and a beta chain variable region comprising a CDR1 beta comprising the sequence MNHEY (SEQ ID NO:84), a CDR2 beta comprising the sequence SMNVEV (SEQ ID NO:98), a CDR3 beta comprising the sequence CASSFAGTDTQYF (SEQ ID NO: 126).

26. The isolated anti-cancer T-cell receptor (TCR) or anti-cancer binding fragment thereof according to any one of claims 1 to 10, wherein the TCR or binding fragment comprises:
(i) an alpha chain variable region comprising a complementarity-determining region (CDR3 alpha) comprising or consisting of any one of:

| (SEQ ID NO: 19) | |
|---|---|
| | CAMSVGFGNVLHC |
| (SEQ ID NO: 20) | |
| | CAALYNTDKLIF |
| (SEQ ID NO: 21) | |
| | CAARGAQKLVF |
| (SEQ ID NO: 22) | |
| | CAATLYNNDMRF |
| (SEQ ID NO: 23) | |
| | CVVSASNGNKLVF |
| (SEQ ID NO: 24) | |
| | CAYLVGSNYQLIW, |
| | or |
| (SEQ ID NO: 25) | |
| | CAFMKPNTGNQFYF, |

or a variant CDR that has 1, 2, 3, 4 or 5 amino acid variations selected from additions, substitutions and deletions with respect thereto; and/or
(ii) a beta chain variable region comprising a complementarity-determining region (CDR3 beta) comprising or consisting of any one of:

| (SEQ ID NO: 53) | |
|---|---|
| | CASSWAGPVEQYF, |
| (SEQ ID NO: 54) | |
| | CASSLGLAGNEQYF, |
| (SEQ ID NO: 55) | |
| | CASSSGLTDTQYF, |
| (SEQ ID NO: 56) | |
| | CASSLGIISGQPQHF, |
| (SEQ ID NO: 57) | |
| | CASSLGGGGYEQYF, |
| (SEQ ID NO: 58) | |
| | CAISPGEGTDTQYF, |
| (SEQ ID NO: 59) | |
| | CASSYTGTLSYEQYF, |
| (SEQ ID NO: 60) | |
| | CASSYVGLGSPLHF, |
| (SEQ ID NO: 61) | |
| | CATSRDMDPEQYF, |
| (SEQ ID NO: 62) | |
| | CASSLGGADTQYF, |
| (SEQ ID NO: 63) | |
| | CASSFLQGTGELFF, |
| (SEQ ID NO: 64) | |
| | CASRVGILSGELFF, |
| (SEQ ID NO: 65) | |
| | CATSRDRGWEQFF, |
| (SEQ ID NO: 66) | |
| | CASSLSGAEAFF, |
| (SEQ ID NO: 67) | |
| | CASSPSWAHETQYF, |
| (SEQ ID NO: 68) | |
| | CASSPSGTVYEKLFF, |
| (SEQ ID NO: 69) | |
| | CSASEGIGQPQHF, |
| (SEQ ID NO: 70) | |
| | CASSPGLTANVLTF, |
| (SEQ ID NO: 71) | |
| | CASSQEQLAGPEQYF, |
| | or |
| (SEQ ID NO: 126) | |
| | CASSFAGTDTQYF, |

or a variant CDR that has 1, 2, 3, 4 or 5 amino acid variations selected from additions, substitutions, and deletions with respect thereto, wherein the TCR or binding fragment binds to the cancer polypeptide antigens Melan-A polypeptide (SEQ ID NO: 72) and IMP2 polypeptide (SEQ ID NO: 74) or antigenic peptide fragments thereof.

27. The isolated anti-cancer T-cell receptor (TCR) or anti-cancer binding fragment thereof according to claim 26 wherein the TCR or binding fragment comprises:
(i) an alpha chain variable region further comprising any one of:
(a). a CDR3 alpha sequence comprising the sequence CAMSVGFGNVLHC (SEQ ID NO: 19) and CDR1 alpha and CDR2 alpha sequence comprising the CDR1 beta and CDR2 beta sequences respectively of TRAV12-3,
(b). a CDR3 alpha sequence comprising the sequence CAALYNTDKLIF (SEQ ID NO: 20) and CDR1 alpha and CDR2 alpha sequence comprising the CDR1 beta and CDR2 beta sequences respectively of TRAV29DV5,
(c). a CDR3 alpha sequence comprising the sequence CAARGAQKLVF (SEQ ID NO: 21) and CDR1 alpha and CDR2 alpha sequence comprising the CDR1 beta and CDR2 beta sequences respectively of TRAV23DV6,
(d). a CDR3 alpha sequence comprising the CAATLYNNDMRF (SEQ ID NO:22) and CDR1 alpha and CDR2 alpha sequence comprising the CDR1 beta and CDR2 beta sequences respectively of TRAV13-1,
(e). a CDR3 alpha sequence comprising the sequence CVVSASNGNKLVF (SEQ ID NO: 23) and CDR1 alpha and CDR2 alpha sequence comprising the CDR1 beta and CDR2 beta sequences respectively of TRAV10,
(f). a CDR3 alpha sequence comprising the sequence CAYLVGSNYQLIW (SEQ ID NO: 24) and CDR1 alpha and CDR2 alpha sequence comprising the CDR1 beta and CDR2 beta sequences respectively of TRAV38-2DV8,
(g). a CDR3 alpha sequence comprising the sequence CAFMKPNTGNQFYF (SEQ ID NO: 25) and CDR1 alpha and CDR2 alpha sequence comprising the CDR1 beta and CDR2 beta sequences respectively of TRAV38-1, or a variant CDR3 alpha that has 1, 2, 3, 4 or 5 amino acid variations selected from additions, substitutions and deletions with respect thereto, and/or a variant CDR1 alpha and/or CDR2 alpha sequence that has 1, 2, or 3 amino acid variations selected from additions, substitutions and deletions with respect thereto; and/or
(ii) a beta chain variable region comprising any one of:
(a). a CDR3 beta sequence comprising the sequence CASSWAGPVEQYF (SEQ ID NO: 53) and CDR1 beta and CDR2 beta sequence comprising the CDR1 beta and CDR2 beta sequences respectively of TRBV12-4,
(b). a CDR3 beta sequence comprising the sequence CASSLGLAGNEQYF (SEQ ID NO: 54) and CDR1 beta and CDR2 beta sequence comprising the CDR1 beta and CDR2 beta sequences respectively of TRBV27,
(c). a CDR3 beta sequence comprising the sequence CASSSGLTDTQYF (SEQ ID NO: 55) TRBV7-3 and CDR1 beta and CDR2 beta sequence comprising the CDR1 beta and CDR2 beta sequences respectively of TRBV7-3,
(d). a CDR3 beta sequence comprising the sequence CASSLGIISGQPQHF (SEQ ID NO: 56) and CDR1 beta and CDR2 beta sequence comprising the CDR1 beta and CDR2 beta sequences respectively of TRBV13,
(e). a CDR3 beta sequence comprising the sequence CASSLGGGGYEQYF (SEQ ID NO: 57) and CDR1 beta and CDR2 beta sequence comprising the CDR1 beta and CDR2 beta sequences respectively of TRBV27,
(f). a CDR3 beta sequence comprising the sequence CAISPGEGTDTQYF (SEQ ID NO: 58) and CDR1 beta and CDR2 beta sequence comprising the CDR1 beta and CDR2 beta sequences respectively of TRBV10-3,
(g). a CDR3 beta sequence comprising the sequence CASSYTGTLSYEQYF (SEQ ID NO: 59) and CDR1 beta and CDR2 beta sequence comprising the CDR1 beta and CDR2 beta sequences respectively of TRBV28,
(h). a CDR3 beta sequence comprising the sequence CASSYVGLGSPLHF (SEQ ID NO: 60) and CDR1 beta and CDR2 beta sequence comprising the CDR1 beta and CDR2 beta sequences respectively of TRBV6-5,
(i). a CDR3 beta sequence comprising the sequence CATSRDMDPEQYF (SEQ ID NO: 61) and CDR1 beta and CDR2 beta sequence comprising the CDR1 beta and CDR2 beta sequences respectively of TRBV15,
(j). a CDR3 beta sequence comprising the sequence CASSLGGADTQYF (SEQ ID NO: 62) and CDR1 beta and CDR2 beta sequence comprising the CDR1 beta and CDR2 beta sequences respectively of TRBV27,
(k). a CDR3 beta sequence comprising the sequence CASSFLQGTGELFF (SEQ ID NO: 63) and CDR1 beta and CDR2 beta sequence comprising the CDR1 beta and CDR2 beta sequences respectively of TRBV27,
(l). a CDR3 beta sequence comprising the sequence CASRVGILSGELFF (SEQ ID NO: 64) and CDR1 beta and CDR2 beta sequence comprising the CDR1 beta and CDR2 beta sequences respectively of TRBV27,
(m). a CDR3 beta sequence comprising the sequence CATSRDRGWEQFF (SEQ ID NO: 65) and CDR1 beta and CDR2 beta sequence comprising the CDR1 beta and CDR2 beta sequences respectively of TRBV15,
(n). a CDR3 beta sequence comprising the sequence CASSLSGAEAFF (SEQ ID NO: 66) and CDR1 beta and CDR2 beta sequence comprising the CDR1 beta and CDR2 beta sequences respectively of TRBV27,
(o). a CDR3 beta sequence comprising the sequence CASSPSWAHETQYF (SEQ ID NO: 67) and CDR1 beta and CDR2 beta sequence comprising the CDR1 beta and CDR2 beta sequences respectively of TRBV27,
(p). a CDR3 beta sequence comprising the sequence CASSPSGTVYEKLFF (SEQ ID NO: 68) and CDR1 beta and CDR2 beta sequence comprising the CDR1 beta and CDR2 beta sequences respectively of TRBV28,
(q). a CDR3 beta sequence comprising the sequence CSASEGIGQPQHF (SEQ ID NO: 69) and CDR1 beta and CDR2 beta sequence comprising the CDR1 beta and CDR2 beta sequences respectively of TRBV20-1,
(r). a CDR3 beta sequence comprising the sequence CASSPGLTANVLTF (SEQ ID NO: 70) and CDR1 beta and CDR2 beta sequence comprising the CDR1 beta and CDR2 beta sequences respectively of TRBV27,
(s). a CDR3 beta sequence comprising the sequence CASSQEQLAGPEQYF (SEQ ID NO: 71) and CDR1 beta and CDR2 beta sequence comprising the CDR1 beta and CDR2 beta sequences respectively of TRBV4-2; or
(t). a CDR3 beta sequence comprising the sequence CASSFAGTDTQYF (SEQ ID NO: 126) and CDR1 beta and CDR2 beta sequence comprising the CDR1 beta and CDR2 beta sequences respectively of TRBV27; or a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

28. The isolated anti-cancer T-cell receptor (TCR) or anti-cancer binding fragment thereof according to claim 26 or 27 wherein the TCR or binding fragment comprises a beta chain variable region comprising any one of:
(a). a CDR3 beta sequence comprising the sequence CASSWAGPVEQYF (SEQ ID NO: 53) and CDR1 beta and CDR2 beta sequences comprising the sequences SGHDY (SEQ ID NO:94) and FNNNVP (SEQ ID NO:111) respectively,
(b). a CDR3 beta sequence comprising the sequence CASSLGLAGNEQYF (SEQ ID NO: 54) and CDR1 beta and CDR2 beta sequences comprising the sequences MNHEY (SEQ ID NO:84) and SMNVEV (SEQ ID NO:98) respectively,
(c). a CDR3 beta sequence comprising the sequence CASSSGLTDTQYF (SEQ ID NO: 55), and CDR1 beta and CDR2 beta sequences comprising the sequences SGHTA (SEQ ID NO:92) and FQGTGA (SEQ ID NO:112) respectively,
(d). a CDR3 beta sequence comprising the sequence CASSLGIISGQPQHF (SEQ ID NO: 56) and CDR1 beta and CDR2 beta sequences comprising the sequences PRHDT (SEQ ID NO:95) and FYEKMQ (SEQ ID NO:113) respectively,
(e). a CDR3 beta sequence comprising the sequence CASSLGGGGYEQYF (SEQ ID NO: 57) and CDR1 beta and CDR2 beta sequences comprising the sequences MNHEY (SEQ ID NO:84) and SMNVEV (SEQ ID NO:98) respectively,
(f). a CDR3 beta sequence comprising the sequence CAISPGEGTDTQYF (SEQ ID NO: 58) and CDR1 beta and CDR2 beta sequences comprising the sequences ENHRY (SEQ ID NO:110) and SYGVKD (SEQ ID NO:114) respectively,
(g). a CDR3 beta sequence comprising the sequence CASSYTGTLSYEQYF (SEQ ID NO: 59) and CDR1 beta and CDR2 beta sequences comprising the sequences MDHEN (SEQ ID NO:85) and SYDVKM (SEQ ID NO:99) respectively,
(h). a CDR3 beta sequence comprising the sequence CASSYVGLGSPLHF (SEQ ID NO: 60) and CDR1 beta and CDR2 beta sequences comprising the sequences MNHEY (SEQ ID NO:84) and SVGAGI (SEQ ID NO:97) respectively,
(i). a CDR3 beta sequence comprising the sequence CATSRDMDPEQYF (SEQ ID NO: 61) and CDR1 beta and CDR2 beta sequences comprising the sequences LNHNV (SEQ ID NO:96) and YYDKDF (SEQ ID NO:115) respectively,
(j). a CDR3 beta sequence comprising the sequence CASSLGGADTQYF (SEQ ID NO: 62) and CDR1 beta and CDR2 beta sequences comprising the sequences MNHEY (SEQ ID NO:84) and SMNVEV (SEQ ID NO:98) respectively,
(k). a CDR3 beta sequence comprising the sequence CASSFLQGTGELFF (SEQ ID NO: 63) and CDR1 beta and CDR2 beta sequences comprising the sequences MNHEY (SEQ ID NO:84) and SMNVEV (SEQ ID NO:98) respectively,
(l). a CDR3 beta sequence comprising the sequence CASRVGILSGELFF (SEQ ID NO: 64) and CDR1 beta and CDR2 beta sequences comprising the sequences MNHEY (SEQ ID NO:84) and SMNVEV (SEQ ID NO:98) respectively,
(m). a CDR3 beta sequence comprising the sequence CATSRDRGWEQFF (SEQ ID NO: 65) and CDR1 beta and CDR2 beta sequences comprising the sequences LNHNV (SEQ ID NO:96) and YYDKDF (SEQ ID NO:115) respectively,
(n). a CDR3 beta sequence comprising the sequence CASSLSGAEAFF (SEQ ID NO: 66) and CDR1 beta and CDR2 beta sequences comprising the sequences MNHEY (SEQ ID NO:84) and SMNVEV (SEQ ID NO:98) respectively,
(o). a CDR3 beta sequence comprising the sequence CASSPSWAHETQYF (SEQ ID NO: 67) and CDR1 beta and CDR2 beta sequences comprising the sequences MNHEY (SEQ ID NO:84) and SMNVEV (SEQ ID NO:98) respectively,
(p). a CDR3 beta sequence comprising the sequence CASSPSGTVYEKLFF (SEQ ID NO: 68) and CDR1 beta and CDR2 beta sequences comprising the sequences MDHEN (SEQ ID NO:85) and SYDVKM (SEQ ID NO:99) respectively,
(q). a CDR3 beta sequence comprising the sequence CSASEGIGQPQHF (SEQ ID NO: 69) and CDR1 beta and CDR2 beta sequences comprising the sequences DFQATT (SEQ ID NO:89) and SNEGSKA (SEQ ID NO:103) respectively,
(r). a CDR3 beta sequence comprising the sequence CASSPGLTANVLTF (SEQ ID NO: 70) and CDR1 beta and CDR2 beta sequences comprising the sequences MNHEY (SEQ ID NO:84) and SMNVEV (SEQ ID NO:98) respectively,
(s). a CDR3 beta sequence comprising the sequence CASSQEQLAGPEQYF (SEQ ID NO: 71) and CDR1 beta and CDR2 beta sequences comprising the sequences LGHNA (SEQ ID NO:90) and YNFKEQ (SEQ ID NO:104) respectively; or
(t). a CDR3 beta sequence comprising the sequence CASSFAGTDTQYF (SEQ ID NO: 126) and CDR1 beta and CDR2 beta sequences comprising the sequences MNHEY (SEQ ID NO:84) and SMNVEV (SEQ ID NO:98), or a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto.

29. The isolated anti-cancer T-cell receptor (TCR) or anti-cancer binding fragment thereof according to any one of claims 1 to 10 or 26 to 28, wherein the TCR or binding fragment comprises either of:
(a) an alpha chain variable region comprising a CDR1 alpha comprising the sequence NSAFQY (SEQ ID NO:131), a CDR2 alpha comprising the sequence TYSSGN (SEQ ID NO:132), and a CDR3 alpha comprising the sequence CAMSVGFGNVLHC (SEQ ID NO:19) and a beta chain variable region comprising a CDR1 beta comprising the sequence MNHEY (SEQ ID NO:84), a CDR2 beta comprising the sequence SMNVEV (SEQ ID NO:98), a CDR3 beta comprising the sequence CASSLGLAGNEQYF (SEQ ID NO:54);
(b) an alpha chain variable region comprising a CDR1 alpha comprising the sequence NTAFDY (SEQ ID NO:133), a CDR2 alpha comprising the sequence IRPDVSE (SEQ ID NO:134), and a CDR3 alpha comprising the sequence CAARGAQKLVF (SEQ ID NO:21), and a beta chain variable region comprising a CDR1 beta comprising the sequence MNHEY (SEQ ID NO:84), a CDR2 beta comprising the sequence SVGAGI (SEQ ID NO:97), a CDR3 beta comprising the sequence CASSYVGLGSPLHF (SEQ ID NO:60).

30. The isolated anti-cancer T-cell receptor (TCR) or anti-cancer binding fragment thereof according to any one of claims 1 to 10, wherein the TCR or binding fragment comprises any of:
(a) an alpha chain variable region comprising a complementarity-determining region (CDR3 alpha) comprising or consisting of: CAVSDNGNKLVF (SEQ ID NO:119) or a variant CDR that has 1, 2, 3, 4 or 5 amino acid variations selected from additions, substitutions and deletions with respect thereto; and/or a beta chain variable region comprising a complementarity-determining region (CDR3 beta) comprising or consisting of CASSWAGPVEQYF (SEQ ID NO:53), or a variant CDR that has 1, 2, 3, 4 or 5 amino acid variations selected from additions, substitutions and deletions with respect thereto,
(b) an alpha chain variable region comprising a CDR3 alpha sequence comprising the sequence CAVSDNGNKLVF (SEQ ID NO: 119) and CDR1 alpha and CDR2 alpha sequence comprising the CDR1 beta and CDR2 beta sequences respectively of TRAV8-6, or a variant CDR3 alpha that has 1, 2, 3, 4 or 5 amino acid variations selected from additions, substitutions and deletions with respect thereto, and/or a variant CDR1 alpha and/or CDR2 alpha sequence that has 1, 2, or 3 amino acid variations selected from additions, substitutions and deletions with respect thereto;
and/or a beta chain variable region comprising a CDR3 beta sequence comprising the sequence CASSWAGPVEQYF (SEQ ID NO:53), and CDR1 beta and CDR2 beta sequence comprising the CDR1 beta and CDR2 beta sequences respectively of TRAV12-4, or a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto,
(c) an alpha chain variable region comprising a CDR3 alpha sequence comprising the sequence CAVSDNGNKLVF (SEQ ID NO: 119), and CDR1 beta and CDR2 alpha sequences comprising the sequences SSVSVY (SEQ ID NO:135) and YLSGSTLV (SEQ ID NO: 136), respectively, or a variant CDR3 alpha that has 1, 2, 3, 4 or 5 amino acid variations selected from additions, substitutions and deletions with respect thereto, and/or a variant CDR1 alpha and/or CDR2 alpha sequence that has 1, 2, or 3 amino acid variations selected from additions, substitutions and deletions with respect thereto; and/or a beta chain variable region comprising a CDR3 beta sequence comprising the sequence CASSWAGPVEQYF (SEQ ID NO:53), and CDR1 beta and CDR2 beta sequences comprising the sequences SGHDY (SEQ ID NO: 94) and FNNNVP (SEQ ID NO: 111), respectively, or a variant CDR3 beta that has 1, 2, 3, 4 or 5 amino acid variations selected from additions, substitutions and deletions with respect thereto, and/or a variant CDR1 beta and/or CDR2 beta sequence that has 1, 2, or 3 amino acid variations selected from additions, substitutions and deletions with respect thereto; or
(d) an alpha chain variable region comprising a CDR3 alpha sequence comprising the sequence CAVSDNGNKLVF (SEQ ID NO: 119), and CDR1 beta and CDR2 alpha sequences comprising the sequences SSVSVY (SEQ ID NO:135) and YLSGSTLV (SEQ ID NO: 136), respectively, and/or a beta chain variable region comprising a CDR3 beta sequence comprising the sequence CASSWAGPVEQYF (SEQ ID NO:53), and CDR1 beta and CDR2 beta sequences comprising the sequences SGHDY (SEQ ID NO:94) and FNNNVP (SEQ ID NO:111), respectively;
wherein the TCR or binding fragment binds to the cancer polypeptide antigens Melan-A polypeptide (SEQ ID NO: 72), BST2 polypeptide (SEQ ID NO: 73) and IMP2 polypeptide (SEQ ID NO: 74) or antigenic peptide fragments thereof,

30. A polynucleotide encoding the TCR or anti-cancer binding fragment according to any one of claims 1 to 29.

31. A vector for delivery of a polynucleotide to cells comprising a polynucleotide according to claim 30.

32. The vector according to claim 31, wherein the vector is a viral vector.

33. An immune cell, such as a T-cell, particularly an engineered immune cell such as an engineered T-cell, expressing the TCR or anti-cancer binding fragment according to any one of claims 1 to 29 or comprising the polynucleotide of claim 30 or vector of either of claim 31 or 32.

34. An immune cell clone, such as a T-cell clone, particularly an engineered immune cell clone such as an engineered T-cell clone, expressing the TCR or anti-cancer binding fragment according to any one of claims 1 to 29.

35. The immune cell, such as a T-cell or immune cell clone, such as a T-cell clone according to either of claim 33 or 34, wherein the cell or cells of the clone are modified by the introduction (e.g. by transduction) of a heterologous polynucleotide according to claim 30 or a vector according to either of claim 31 or 32.

36. An ex vivo process comprising (i) obtaining immune cells, particularly T-cells from a patient, (ii) optionally expanding the immune cells, particularly T-cells (iii) introducing a heterologous polynucleotide according to claim 30 or a vector according to either of claim 31 or 32 into the immune cells, particularly T-cells to produce modified immune cells, particularly T-cells which express a TCR or an anti-cancer binding fragment of a TCR according to any one of claims 1 to 29; and (iii) reintroducing said modified immune cells, particularly T-cells into the patient.

37. Immune cells, particularly T-cells, for use in treating cancer in a patient wherein the immune cells, particularly T-cells are immune cells which have been obtained from said patient and a heterologous polynucleotide according to claim 30 or a vector according to either of claim 31 or 32 has been introduced into the immune cells such that the immune cells express an anti-cancer TCR or an anti-cancer binding fragment of a TCR according to any one of claims 1 to 29.

38. A method of treatment of cancer comprising administering to a patient in need thereof immune cells, particularly T-cells wherein the immune cells, particularly T-cells are immune cells, particularly T-cells which have been obtained from said patient and a heterologous polynucleotide according to claim 30 or a vector according to any one of claims 31 to 32 has been introduced into the immune cells, particularly T-cells, such that the immune cells, particularly T-cells express an anti-cancer TCR or an anti-cancer binding fragment of a TCR according to any one of claims 1 to 29.

39. A pharmaceutical composition comprising the TCR or anti-cancer binding fragment according to any one of claims 1 to 29, the polynucleotide according to claim 30, a vector according to either of claims 31 to 32, the immune cell or the immune cell clone, particularly the T-cell or the T-cell clone according to any one of claims 33 to 35 and a pharmaceutically acceptable carrier.

40. A pharmaceutical composition according to claim 39, wherein the pharmaceutical composition is formulated under sterile conditions and is suitable for parenteral administration.

41. A bispecific construct comprising the TCR or anti-cancer binding fragment according to any one of claims 1 to 29 and an immune cell activating component or ligand that binds to and activates an immune cell.

42. A bispecific construct according to claim 41, wherein the immune cell activating component or ligand that binds to and activates an immune cell binds to CD3.

43. A fusion protein comprising the TCR or anti-cancer binding fragment according to any one of claims 1 to 29 and a heterologous protein.

44. A method of treating cancer in a subject comprising administering a therapeutically effective amount of the immune cell, immune cell clone, T-cell or T-cell clone according to any one of claims 33 to 35, pharmaceutical composition according to either of claims 39 to 40, bispecific construct according to claim 41 or 42 or fusion protein according to claim 43, to the subject.

45. The use according to claim 37 or method according to either of claim 38 or 44 wherein said cancer is selected from lung, melanoma (e.g. skin melanoma), bone, breast, blood (e.g. leukemia), prostate, kidney, bladder, cervical, ovarian and colorectal cancers.

46. The use according to claim 37 or method according to either of claim 38 or 44 wherein, wherein the immune cell, immune cell clone, T-cell, T-cell clone, pharmaceutical composition, bispecific construct or fusion protein is administered separately, simultaneously or sequentially with an anti-cancer agent.

47. A pharmaceutical composition comprising:
a) the immune cell, immune cell clone, T-cell or T-cell clone according to any one of claims 33 to 35, the pharmaceutical composition according to either of claim 39 or 40, bispecific construct according to either one of claim 41 or 42 or the fusion protein according to claim 43; and
b) an anti-cancer agent.
